(19) United States
(12) Patent Application Publication
(10) Pub. No.: US 2008/0021058 A1
(43) Pub. Date: Jan. 24, 2008

(54) Pyrazolo '3,4-B! Pyridine Compounds And Their Use As Phosphodiesterase Type 4(Pde4) Inhibitors

(76) Inventors: David George Allen, Herfordshire (GB); Diane Mary Coe, Hertfordshire (GB); Caroline Mary Cook, Hertfordshire (GB); Michael Dennis Dowle, Hertfordshire (GB); Christopher David Edlin, Hertfordshire (GB); Julie Nicole Hamblin, Hertfordshire (GB); Martin Redpath Johnson, Hertfordshire (GB); Paul Spence Jones, Hertfordshire (GB); Mika Kristian Lindvall, Emeryville, CA (US); Charlotte Jane Mitchell, Hertfordshire (GB); Alison Judith Redgrave, Hertfordshire (GB); Naimisha Trivedi, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/598,940

(22) PCT Filed: Mar. 15, 2005
(86) PCT No.: PCT/GB05/00983

§ 371 Date: Jul. 11, 2007

(30) Foreign Application Priority Data
Mar. 16, 2004 (GB) 0405933.3

(51) Int. Cl. A61K 31/437; A61P 29/00; A61P 37/08; C07D 221/04

(52) U.S. Cl. 514/303; 546/119

(57) ABSTRACT

The invention provides a compound of formula (I) or a salt thereof: 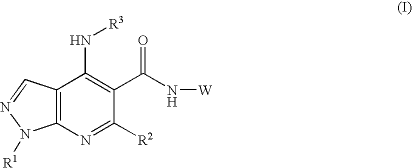
wherein W is Ar, —CR⁴R⁵Ar or a group (y) or (y1), wherein Ar is (x) or (z): 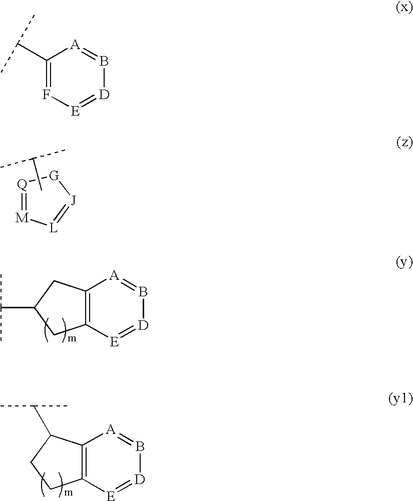
R¹ is C₁₋₄alkyl, C₁₋₃fluoroalkyl or —CH₂CH₂OH. R² is C₂₋₆alkyl, C₃₋₆cycloalkyl or —(CH₂)ₙ4-C₃₋₆cycloalkyl; and R³ is optionally substituted C₃₋₈cycloalkyl, optionally substituted mono-unsaturated-C₅₋₇cycloalkenyl, an optionally substituted heterocyclic group (aa), (bb) or (cc) (in which Y is O, S, SO₂, or NR¹⁰), or a bicyclic group (ee): 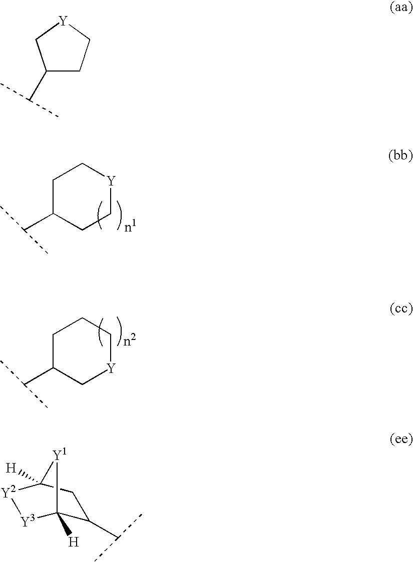 These compounds are PDE4 inhibitors.

The present invention relates to pyrazolo[3,4-b]pyridine compounds, processes for their preparation, intermediates usable in these processes, and pharmaceutical compositions containing the compounds. The invention also relates to the use of the pyrazolo[3,4-b]pyridine compounds in therapy, for example as inhibitors of phosphodiesterase type IV (PDE4) and/or for the treatment and/or prophylaxis of inflammatory and/or allergic diseases such as chronic obstructive pulmonary disease (COPD), asthma, rheumatoid arthritis or allergic rhinitis.

### BACKGROUND TO THE INVENTION

U.S. Pat. No. 3,979,399, U.S. Pat. No. 3,840,546, and U.S. Pat. No. 3,966,746 (E.R. Squibb & Sons) disclose 4-amino derivatives of pyrazolo[3,4-b]pyridine-5-carboxamides wherein the 4-amino group NR₃R₄ can be an acyclic amino group wherein R₃ and R₄ may each be hydrogen, lower alkyl (e.g. butyl), phenyl, etc.; NR₃R₄ can alternatively be a 3-6-membered heterocyclic group such as pyrrolidino, piperidino and piperazino. The compounds are disclosed as central nervous system depressants useful as ataractic, analgesic and hypotensive agents.

U.S. Pat. No. 3,925,388, U.S. Pat. No. 3,856,799, U.S. Pat. No. 3,833,594 and U.S. Pat. No. 3,755,340 (E.R. Squibb & Sons) disclose 4-amino derivatives of pyrazolo[3,4-b]pyridine-5-carboxylic acids and esters. The 4-amino group NR₃R₄ can be an acyclic amino group wherein R₃ and R₄ may each be hydrogen, lower alkyl (e.g. butyl), phenyl, etc.; NR₃R₄ can alternatively be a 5-6-membered heterocyclic group in which an additional nitrogen is present such as pyrrolidino, piperidino, pyrazolyl, pyrimidinyl, pyridazinyl or piperazinyl. The compounds are mentioned as being central nervous system depressants useful as ataractic agents or tranquilisers, as having antiinflammatory and analgesic properties. The compounds are mentioned as increasing the intracellular concentration of adenosine-3′,5′-cyclic monophosphate and for alleviating the symptoms of asthma.

H. Hoehn et al., J. Heterocycl. Chem., 1972, 9(2), 235-253 discloses a series of 1H-pyrazolo[3,4-b]pyridine-5-carboxylic acid derivatives with 4-hydroxy, 4-chloro, 4-alkoxy, 4-hydrazino, and 4-amino substituents.

CA 1003419, CH 553 799 and T. Denzel, Archiv der Pharmazie, 1974, 307(3), 177-186 disclose 4,5-disubstituted 1H-pyrazolo[3,4-b]pyridines unsubstituted at the 1-position.

Japanese laid-open patent application JP-2002-20386-A (Ono Yakuhin Kogyo K K) published on 23 Jan. 2002 discloses pyrazolopyridine compounds of the following formula: 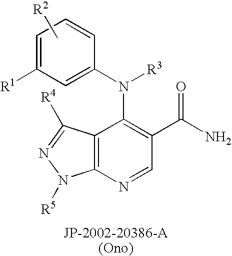
wherein R¹ denotes 1) a group —OR⁶, 2) a group —SR⁷, 3) a C2-8 alkynyl group, 4) a nitro group, 5) a cyano group, 6) a C1-8 alkyl group substituted by a hydroxy group or a C1-8 alkoxy group, 7) a phenyl group, 8) a group —C(O)R⁸, 9) a group —SO₂NR⁹R¹⁰, 10) a group —NR¹¹SO₂R¹², 11) a group —NR¹³C(O)R¹⁴ or 12) a group —CH═NR¹⁵. R⁶ and R⁷ denote i) a hydrogen atom, ii) a C1-8 alkyl group, iii) a C1-8 alkyl group substituted by a C1-8 alkoxy group, iv) a trihalomethyl group, v) a C3-7 cycloalkyl group, vi) a C1-8 alkyl group substituted by a phenyl group or vii) a 3-15 membered mono-, di- or tricyclic hetero ring containing 1-4 nitrogen atoms, 1-3 oxygen atoms and/or 1-3 sulphur atoms. R² denotes 1) a hydrogen atom or 2) a C1-8 alkoxy group. R³ denotes 1) a hydrogen atom or 2) a C1-8 alkyl group. R⁴ denotes 1) a hydrogen atom, 2) a C1-8 alkyl group, 3) a C3-7 cycloalkyl group, 4) a C1-8 alkyl group substituted by a C3-7 cycloalkyl group, 5) a phenyl group which may be substituted by 1-3 halogen atoms or 6) a 3-15 membered mono-, di- or tricyclic hetero ring containing 1-4 nitrogen atoms, 1-3 oxygen atoms and/or 1-3 sulphur atoms. R⁵ denotes 1) a hydrogen atom, 2) a C1-8 alkyl group, 3) a C3-7 cycloalkyl group, 4) a C1-8 alkyl group substituted by a C3-7 cycloalkyl group or 5) a phenyl group which may be substituted by 1-3 substituents. In group R³, a hydrogen atom is preferred. In group R⁴, methyl, ethyl, cyclopropyl, cyclobutyl or cyclopentyl are preferred. The compounds of JP-2002-20386-A are stated as having PDE4 inhibitory activity and as being useful in the prevention and/or treatment of inflammatory diseases and many other diseases,

1,3-Dimethyl-4-(arylamino)-pyrazolo[3,4-b]pyridines with a 5-C(O)NH₂ substituent similar or identical to those in JP-2002-20386-A were disclosed as orally active PDE4 inhibitors by authors from Ono Pharmaceutical Co. in: H. Ochiai et al., Bioorg. Med. Chem. Lett., 5th Jan. 2004 issue, vol. 14(1), pp. 29-32 (available on or before 4th Dec. 2003 from the Web version of the journal: “articles in press”).

EP 0 076 035 A1 (ICI Americas) discloses pyrazolo[3,4-b]pyridine derivatives as central nervous system depressants useful as tranquilisers or ataractic agents for the relief of anxiety and tension states.

The compound cartazolate, ethyl 4-(n-butylamino)-1-ethyl-1H-pyrazolo[3,4-b]-pyridine-5-carboxylate, is known. J. W. Daly et al., Med. Chem. Res., 1994, 4, 293-306 and D. Shi et al., Drug Development Research, 1997, 42, 41-56 disclose a series of 4-(amino)substituted 1H-pyrazolo[3,4-b]pyridine-5-carboxylic acid derivatives, including ethyl 4-cyclopentylamino-1-methyl-1H-pyrazolo[3,4-b]pyridine-5-carboxylate, and their affinities and antagonist activities at A₁- and A_{2A}-adenosine receptors, and the latter paper discloses their affinities at various binding sites of the GABA_{A}-receptor channel. S. Schenone et al., Bioorg. Med. Chem. Lett., 2001, 11, 2529-2531, and F. Bondavalli et al., J. Med. Chem., 2002, vol. 45 (Issue 22, 24 Oct. 2002, allegedly published on Web Sep. 24, 2002), pp. 4875-4887 disclose a series of 4-amino-1-(2-chloro-2-phenylethyl)-1H-pyrazolo[3,4-b]pyridine-5-carboxylic acid ethyl esters as A₁-adenosine receptor ligands.

WO 02/060900 A2 appears to disclose, as MCP-1 antagonists for treatment of allergic, inflammatory or autoimmune disorders or diseases, a series of bicyclic heterocyclic compounds with a —C(O)—NR⁴—C(O)—NR⁵R⁶ substituent, including isoxazolo[5,4-b]pyridines and 1H-pyrazolo[3,4-b]pyridines (named as pyrazolo[5,4-b]pyridines) with the —C(O)—NR⁴—C(O)—NR⁵R⁶ group as the 5-substituent and optionally substituted at the 1-, 3-, 4-, and/or 6-positions. Bicyclic heterocyclic compounds with a —C(O)NH₂ substituent instead of the —C(O)—NR⁴—C(O)—NR⁵R⁶ substituent are alleged to be disclosed in WO 02/060900 as intermediates in the synthesis of the —C(O)—NR⁴—C(O)—NR⁵R⁶ substituted compounds.

WO 00/15222 (Bristol-Myers Squibb) discloses inter alia pyrazolo[3,4-b]pyridines having inter alia a C(O)—X₁ group at the 5-position and a group E₁ at the 4-position of the ring system. Amongst other things, X₁ can for example be —OR₉, —N(R₉)(R₁₀) or —N(R₅)(-A₂-R₂), and E₁ can for example be —NH-A₁-cycloalkyl, —NH-A₁-substituted cycloalkyl, or —NH-A₁-heterocyclo; wherein A₁ is an alkylene or substituted alkylene bridge of 1 to 10 carbons and A₂ can for example be a direct bond or an alkylene or substituted alkylene bridge of 1 to 10 carbons. The compounds are disclosed as being useful as inhibitors of cGMP phosphodiesterase, especially PDE type V, and in the treatment of various cGMP-associated conditions such as erectile dysfunction. Compounds with a cycloalkyl or heterocyclo group directly attached to —NH— at the 4-position of the pyrazolo[3,4-b]pyridine ring system and/or having PDE4 inhibitory activity do not appear to be disclosed in WO 00/15222.

Copending patent application PCT/EP03/11814, filed on 12 Sep. 2003 in the name of Glaxo Group Limited, and incorporated herein by reference, discloses pyrazolo[3,4-b]pyridine compounds or salts thereof with a 4-NHR³ group and a 5-C(O)—X group, according to this formula (I): 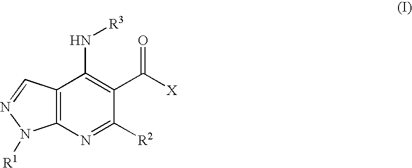
wherein:

R¹ is C₁₋₄alkyl, C₁₋₃fluoroalkyl, —CH₂CH₂OH or —CH₂CH₂CO₂C₁₋₂alkyl;

R² is a hydrogen atom (H), methyl or C₁fluoroalkyl;

R³ is optionally substituted C₃₋₈cycloalkyl or optionally substituted mono-unsaturated-C₅₋₇cycloalkenyl or an optionally substituted heterocyclic group of sub-formula (aa), (bb) or (cc); 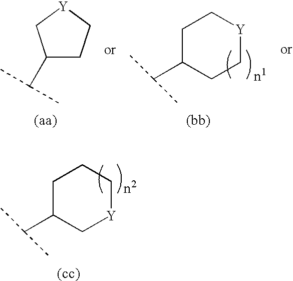
in which n¹ and n² independently are 1 or 2; and in which Y is O, S, SO₂, or NR¹⁰; 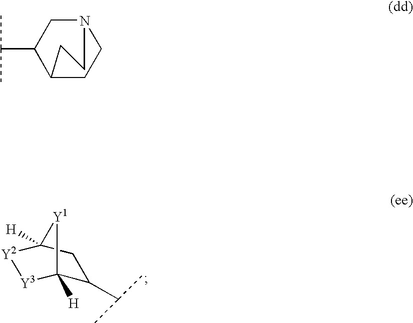
or R³ is a bicyclic group (dd) or (ee): (dd) (ee):
and wherein X is NR⁴R⁵ or OR^{5a}.

In PCT/EP03/11814, R⁴ is a hydrogen atom (H); C₁₋₆alkyl; C₁₋₃fluoroalkyl; or C₂₋₆alkyl substituted by one substituent R¹¹.

In PCT/EP03/11814, R⁵ can be: a hydrogen atom (H); C₁₋₈alkyl; C₁₋₈ fluoroalkyl; C₃₋₈cycloalkyl optionally substituted by a C₁₋₂alkyl group; —(CH₂)ₙ⁴—C₃₋₈cycloalkyl optionally substituted, in the —(CH₂)ₙ⁴— moiety or in the C₃₋₈cycloalkyl moiety, by a C₁₋₂alkyl group, wherein n⁴ is 1, 2 or 3; C₂₋₆alkyl substituted by one or two independent substituents R¹¹; —(CH₂)ₙ¹¹—C(O)R¹⁶; —(CH₂)ₙ¹²—C(O)NR¹²R¹³; —CHR¹⁹—C(O)NR¹²R¹³; —(CH₂)ₙ¹²—C(O)OR¹⁶; —(CH₂)ₙ¹²—C(O)OH; —CHR¹⁹—C(O)OR¹⁶; —CHR¹⁹—C(O)OH; —(CH₂)ₙ¹²—SO₂—NR¹²R¹³; —(CH₂)ₙ¹²—SO₂R¹⁶; or —(CH₂)ₙ¹²—CN; —(CH₂)ₙ¹³-Het; or optionally substituted phenyl.

Alternatively, in PCT/EP03/11814, R⁵ can have the sub-formula (x), (y), (y1) or (z): 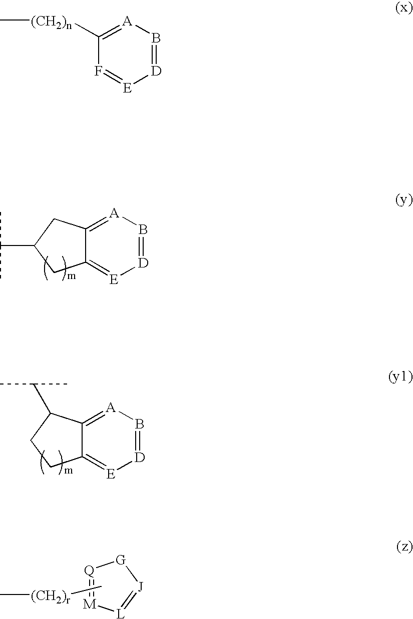
wherein in sub-formula (x), n=0, 1 or 2; in sub-formula (y) and (y1), m=1 or 2; and in sub-formula (z), r=0, 1 or 2; and wherein in sub-formula (x) and (y) and (y1), none, one or two of A, B, D, E and F are independently nitrogen or nitrogen-oxide (N⁺—O⁻) provided that no more than one of A, B, D, E and F is nitrogen-oxide, and the remaining of A, B, D, E and F are independently CH or CR⁶; and provided that when n is 0 in sub-formula (x) then one or two of A, B, D, E and F are independently nitrogen or nitrogen-oxide (N⁺—O⁻) and no more than one of A, B, D, E and F is nitrogen-oxide;

In PCT/EP03/11814, each R⁶, independently of any other R⁶ present, is: a halogen atom; C₁₋₆alkyl; C₁₋₄fluoroalkyl; C₁₋₄alkoxy; C₁₋₂fluoroalkoxy; C₃₋₆cycloalkyloxy; —C(O)R^{16a}; —C(O)OR³⁰; —S(O)₂—R^{16a}; R^{16a}—S(O)₂—NR^{15a}—; R⁷R⁸N—S(O)₂—; C₁₋₂alkyl-C(O)—R^{15a}N—S(O)₂—; C₁₋₄alkyl-S(O)—; Ph-S(O)—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—NR⁷R⁸; —CH₂—CH₂—NR⁷R⁸; —CH₂—C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano (CN); Ar^{5b}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy;

or two adjacent R⁶ taken together can be —O—(CMe₂)-O— or —O—(CH₂)ₙ¹⁴—O— where n¹⁴ is 1 or 2.

In PCT/EP03/11814, in sub-formula (z), G is O or S or NR⁹ wherein R⁹ is a hydrogen atom (H), C₁₋₄alkyl or C₁₋₄fluoroalkyl; none, one, two or three of J, L, M and Q are nitrogen; and the remaining of J, L, M and Q are independently CH or CR⁶ where R⁶, independently of any other R⁶ present, is as defined therein.

The pyrazolo[3,4-b]pyridine compounds of formula (I) and salts thereof disclosed in PCT/EP03/11814 are disclosed as being inhibitors of phosphodiesterase type IV (PDE4), and as being useful for the treatment and/or prophylaxis of an inflammatory and/or allergic diseases such as chronic obstructive pulmonary disease (COPD), asthma, rheumatoid arthritis, or allergic rhinitis.

The Invention

We have now found new pyrazolo[3,4-b]pyridine compounds, having a —C(O)—NH—W substituent at the 5-position of the pyrazolo[3,4-b]pyridine ring system, which compounds inhibit phosphodiesterase type IV (PDE4).

The present invention therefore provides a compound of formula (I) or a salt thereof (in particular, a pharmaceutically acceptable salt thereof): 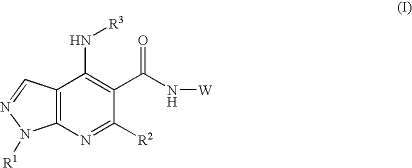
wherein:

W is Ar, —CR⁴R⁵Ar or a group (y) or (y1) wherein: 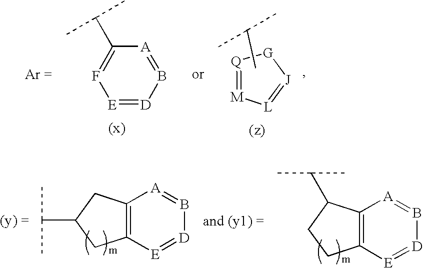
wherein m=1 or 2

R¹ is C₁₋₄alkyl, C₁₋₃fluoroalkyl, or —CH₂CH₂OH;

R² is C₂₋₆alkyl, C₃₋₆cycloalkyl or —(CH₂)ₙ⁴C₃₋₆cycloalkyl, wherein n⁴ is 1 or 2;

R³ is optionally substituted C₃₋₈cycloalkyl or optionally substituted mono-unsaturated-C₅₋₇cycloalkenyl or an optionally substituted heterocyclic group of sub-formula (aa), (bb) or (cc); 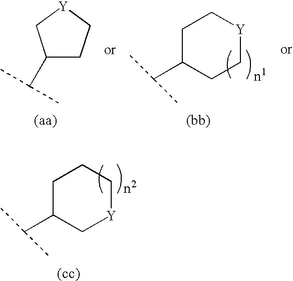
in which n¹ and n² independently are 1 or 2; and in which Y is O, S, SO₂, or NR¹⁰; where R¹⁰ is a hydrogen atom (H), C₁₋₂alkyl, C₁₋₂fluoroalkyl, CH₂C(O)NH₂, C(O)NH₂, C(O)NHMe, C(O)—C₁₋₂alkyl, C(O)—C₁fluoroalkyl or —C(O)—CH₂O—C₁₋₂alkyl;

and wherein in R³ the C₃₋₈cycloalkyl or the heterocyclic group of sub-formula (aa), (bb) or (cc) is optionally substituted on a ring carbon with one or two substituents independently being oxo (═O); OH; C₁₋₂alkoxy; C₁₋₂fluoroalkoxy; NHR²¹ wherein R²¹ wherein R²¹ is a hydrogen atom (H) or C₁₋₄ straight-chain alkyl; C₁₋₂alkyl; C₁₋₂fluoroalkyl; —CH₂OH; —CH₂CH₂OH; —CH₂NHR²² wherein R²² is H or C₁₋₂alkyl; —C(O)OR²³ wherein R²³ is H or C₁₋₂alkyl; —C(O)NHR²⁴ wherein R²⁴ is H or C₁₋₂alkyl; —C(O)R²⁵ wherein R²⁵ is C₁₋₂alkyl; fluoro; hydroxyimino (═N—OH); or (C₁₋₄alkoxy)imino (═N—OR²⁶ where R²⁶ is C₁₋₄alkyl); and wherein any OH, alkoxy, fluoroalkoxy or NHR²¹ substituent is not substituted at the R³ ring carbon attached (bonded) to the —NH— group of formula (I) and is not substituted at either R³ ring carbon bonded to the Y group of the heterocyclic group (aa), (bb) or (cc);

and wherein, when R³ is optionally substituted mono-unsaturated-C₅₋₇cycloalkenyl, then the cycloalkenyl is optionally substituted with one substituent being fluoro or C₁₋₂alkyl or two substituents independently being fluoro or methyl, and the R³ ring carbon bonded to the —NH— group of formula (I) does not partake in the cycloalkenyl double bond;

or R³ is a bicyclic group of sub-formula (ee): 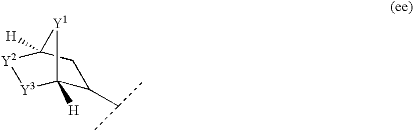
wherein Y¹, Y² and Y³ independently are CH₂ or oxygen (O) provided that no more than one of Y¹, Y² and Y³ is oxygen (O);

and wherein:

R⁴ and R⁵ are independently a hydrogen atom (H), methyl, ethyl, n-propyl, isopropyl, C₁₋₂fluoroalkyl, cyclopropyl, —CH₂OR^{4a}, —CH(Me)OR^{4a}, or —CH₂CH₂OR^{4a}, wherein R^{4a} is a hydrogen atom (H), methyl (Me), or C₁fluoroalkyl such as CF₃ or CHF₂.

and wherein, in sub-formula (x) (y) and (y1):

A is C—R^{6A}, nitrogen (N) or nitrogen-oxide (N⁺—O⁻)), B is C—R^{6B}, nitrogen (N) or nitrogen-oxide (N⁺—O⁻)), D is C—R^{6D}, nitrogen (N) or nitrogen-oxide (N⁺—O⁻)), E is C—R^{6E}, nitrogen (N) or nitrogen-oxide (N⁺—O⁻)), F is C—R^{6F}, nitrogen (N) or nitrogen-oxide (N⁺—O⁻)),

wherein, R^{6A}, R^{6B}, R^{6D}, R^{6E} and R^{6F} independently are: a hydrogen atom (H), a halogen atom; C₁₋₆alkyl (e.g. C₁₋₄alkyl or C₁₋₂alkyl); C₁₋₄fluoroalkyl (e.g. C₁₋₂fluoroalkyl); C₃₋₆cycloalkyl; C₁₋₄alkoxy (e.g. C₁₋₂alkoxy); C₁₋₂fluoroalkoxy; C₃₋₆cycloalkyloxy; —C(O)R^{16a}; —C(O)OR³⁰; —S(O)₂—R^{16a} (e.g. C₁₋₂alkyl-S(O)₂—); R^{16a}—S(O)₂—NR^{15a}— (e.g. C₁₋₂alkyl-S(O)₂—NH—); R⁷R⁸N—S(O)₂—; C₁₋₂alkyl-C(O)—R^{15a}N—S(O)₂—; C₁₋₄alkyl-S(O)—, Ph-S(O)—, R⁷R⁸N—CO—; —NR¹⁵—C(O)R^{16a}; R⁷R⁸N; nitro(—NO₂); OH (including any tautomer thereof); C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—R⁷R⁸; —CH₂—CH₂—NR⁷R⁸; —CH₂—C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano(—CN); Ar^{5b}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy;

and/or two adjacent groups selected from R^{6A}, R^{6B}, R^{6D}, R^{6E} and R^{6F} are taken together and are: —CH═CH—CH═CH₂—, —(CH₂)ₙ^{14a}— where n^{14a} is 3, 4 or 5 (e.g. 3 or 4), —O—(CMe₂)—O—, —O—(CH₂)ₙ^{14b}—O— where n^{14b} is 1 or 2; —CH═CH—NR^{15b}—; —N═CH—NR^{15b}—; —CH═N—NR^{15b}—; —N═N—NR^{15b}—; —CH═CH—O—; —N═CH—O—; —CH═CH—S—; or —N═CH—S—; wherein R^{15b} is H or C₁₋₂alkyl;

provided that:
two or more of A, B, D, E and F are independently C—H (carbon-hydrogen), C—F (carbon-fluorine), nitrogen (N), or nitrogen-oxide (N⁺—O⁻));
and no more than two of A, B, D, E and F are independently nitrogen or nitrogen-oxide (N⁺—O⁻)),
and no more than one of A, B, D, E and F is nitrogen-oxide (N⁺—O⁻));

and wherein, in sub-formula (z):

G is O or S or NR⁹ wherein R⁹ is a hydrogen atom (H), C₁₋₄alkyl, or C₁₋₂fluoroalkyl; J is C—R^{6J}, C-[connection point to formula (I)], or nitrogen (N), L is C—R^{6L}, C-[connection point to formula (I)], or nitrogen (N), M is C—R^{6M}, C-[connection point to formula (I)], or nitrogen (N), Q is C—R^{6Q}, C-[connection point to formula (I)], or nitrogen (N),

wherein, R^{6J}, R^{6L}, R^{6M} and R^{6Q} independently are: a hydrogen atom (H), a halogen atom; C₁₋₄alkyl (e.g. C₁₋₂alkyl); C₁₋₃fluoroalkyl (e.g. C₁₋₂fluoroalkyl); C₃₋₆cycloalkyl; C₁₋₄alkoxy (e.g. C₁₋₂alkoxy); C₁₋₂fluoroalkoxy; C₃₋₆cycloalkyloxy; OH (including any tautomer thereof); or phenyl optionally substituted by one or two substituents independently being fluoro, chloro, C₁₋₂alkyl, C₁fluoroalkyl, C₁₋₂alkoxy or C₁fluoroalkoxy;

provided that:
two or more of J, L, M and Q are independently C—H, C—F, C—C₁₋₂alkyl (e.g. C-Me), C-[connection point to formula (I)], or nitrogen (N);
and no more than three of J, L, M and Q are nitrogen (N);
   and wherein:

R⁷ and R⁸ are independently a hydrogen atom (H); C₁₋₄alkyl (e.g. C₁₋₂alkyl such as methyl); C₃₋₆cycloalkyl; or phenyl optionally substituted by one or two substituents independently being: fluoro, chloro, C₁₋₂alkyl, C₁fluoroalkyl, C₁₋₂alkoxy or C₁fluoroalkoxy;

or R⁷ and R⁸ together are —(CH₂)ₙ⁶— or —C(O)—(CH₂)ₙ⁷— or —C(O)—(CH₂)ₙ¹⁰—C(O)— or —(CH₂)ₙ⁸—X⁷—(CH₂)ₙ⁹— or —C(O)—X⁷—(CH₂)ₙ¹⁰— in which: n⁶ is 3, 4, 5 or 6 (suitably n⁶ is 4 or 5), n⁷ is 2, 3, 4, or 5 (suitably n⁷ is 3 or 4), n⁸ and n⁹ and n¹⁰ independently are 2 or 3 (suitably independently 2), and X⁷ is O or NR¹⁴;

R^{7a} is a hydrogen atom (H) or C₁₋₄alkyl (suitably H or C₁₋₂alkyl, more suitably H or methyl);

R^{8a} is a hydrogen atom (H) or methyl (suitably H);

R¹⁴, R¹⁷ and R^{17a} independently are: a hydrogen atom (H); C₁₋₄alkyl (e.g. C₁₋₂alkyl); C₁₋₂fluoroalkyl (e.g. CF₃); cyclopropyl; —C(O)—C₁₋₄alkyl (e.g. —C(O)Me);

—C(O)NR^{7a}R^{8a} (e.g. —C(O)NH₂); or —S(O)₂—C₁₋₄alkyl (e.g. —S(O)₂Me) (preferably, R¹⁴, R¹⁷ and/or R^{17a} independently is/are: H; C₁₋₂alkyl; or —C(O)Me);

R^{15a}, independent of other R^{15a}, is a hydrogen atom (H) or C₁₋₄alkyl (e.g. H, ^{t}Bu or C₁₋₂alkyl such as methyl; preferably R^{15a} is H or C₁₋₂alkyl, more preferably H);

R^{16a} is:

C₁₋₆alkyl (e.g. C₁₋₄alkyl or C₁₋₂alkyl);

C₃₋₆cycloalkyl (e.g. C₅₋₆cycloalkyl) optionally substituted by one oxo (═O), OH or C₁₋₂alkyl substituent (e.g. optionally substituted at the 3- or 4-position of a C₅₋₆cycloalkyl ring; and/or preferably unsubstituted C₃₋₆cycloalkyl);

C₃₋₆cycloalkyl-CH₂— (e.g. C₅₋₆cycloalkyl-CH₂—);

pyridinyl (e.g. pyridin-2-yl) optionally substituted on a ring carbon atom by one of: a halogen atom, C₁₋₂alkyl, C₁fluoroalkyl, C₁₋₂alkoxy or C₁fluoroalkoxy;

Ar^{5c};

phenyl optionally substituted by one or two substituents independently being: a halogen atom, C₁₋₂alkyl, C₁fluoroalkyl, C₁₋₂alkoxy or C₁fluoroalkoxy;

benzyl optionally substituted on its ring by one or two substituents independently being: a halogen atom, C₁₋₂alkyl, C₁fluoroalkyl, C₁₋₂alkoxy or C₁fluoroalkoxy; or

a 4-, 5-, 6- or 7-membered saturated heterocyclic ring connected at a ring-carbon and containing one or two ring-hetero-atoms independently selected from O, S, and N;

wherein any ring-nitrogens which are present are present as NR²⁷ where R²⁷ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen;

R³⁰, independent of other R³⁰, is a hydrogen atom (H), C₁₋₄alkyl or C₃₋₆cycloalkyl;

Ar^{5b} and Ar^{5c} independently is/are a 5-membered aromatic heterocyclic ring containing one O, S or NR^{15a} in the 5-membered ring, wherein the 5-membered ring can optionally additionally contain one or two N atoms, and wherein the heterocyclic ring is optionally substituted on a ring carbon atom by one of: a halogen atom, C₁₋₂alkyl, C₁fluoroalkyl, —CH₂OH, —CH₂—OC₁₋₂alkyl, OH (including the keto tautomer thereof) or —CH₂—NR²⁸R²⁹ wherein R²⁸ and R²⁹ independently are H or methyl; and

Het¹, is a 4-, 5-, 6- or 7-membered saturated heterocyclic ring connected at a ring-carbon and containing one or two ring-hetero-atoms independently selected from O, S, and N; wherein any ring-nitrogens which are present are present as NR³¹ where R³¹ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen.

In compounds, for example in the compounds of formula (I) (or formula (IA) or formula (IB), see later), an “alkyl” group or moiety may be straight-chain or branched. Alkyl groups, for example C₁₋₈alkyl or C₁₋₆alkyl or C₁₋₄alkyl or C₁₋₃alkyl or C₁₋₂alkyl, which may be employed include C₁₋₆alkyl or C₁₋₄alkyl or C₁₋₃alkyl or C₁₋₂alkyl such as methyl, ethyl, n-propyl, n-butyl, n-pentyl, or n-hexyl or any branched isomers thereof such as isopropyl, t-butyl, sec-butyl, isobutyl, 3-methylbutan-2-yl, 2-ethylbutan-1-yl, or the like.

A corresponding meaning is intended for “alkoxy”, “alkylene”, and like terms derived from alkyl. For example, “alkoxy” such as C₁₋₆alkoxy or C₁₋₄alkoxy or C₁₋₂alkoxy includes methoxy, ethoxy, propyloxy, and oxy derivatives of the alkyls listed above. “Alkylsulfonyl” such as C₁₋₄alkylsulfonyl includes methylsulfonyl (methanesulfonyl), ethylsulfonyl, and others derived from the alkyls listed above. “Alkylsulfonyloxy” such as C₁₋₄alkylsulfonyloxy includes methanesulfonyloxy (methylsulfonyloxy), ethanesulfonyloxy, et al. “Cycloalkyl”, for example C₃₋₈cycloalkyl, includes cyclopropyl, cyclobutyl, cyclopentyl, cyclohexyl, cycloheptyl, cyclooctyl, and the like. Preferably, a C₃₋₈cycloalkyl group is C₃₋₆cycloalkyl or C₅₋₆cycloalkyl, that is contains a 3-6 membered or 5-6 membered carbocyclic ring.

“Fluoroalkyl” includes alkyl groups with one, two, three, four, five or more fluorine substituents, for example C₁₋₄fluoroalkyl or C₁₋₃fluoroalkyl or C₁₋₂fluoroalkyl such as monofluoromethyl, difluoromethyl, trifluoromethyl, pentafluoroethyl, 2,2,2-trifluoroethyl (CF₃CH₂—), 2,2-difluoroethyl (CHF₂CH₂—), 2-fluoroethyl (CH₂FCH₂—), etc. “Fluoroalkoxy” includes C₁₋₄fluoroalkoxy or C₁₋₂fluoroalkoxy such as trifluoromethoxy, pentafluoroethoxy, monofluoromethoxy, difluoromethoxy, etc. “Fluoroalkylsulfonyl” such as C₁₋₄fluoroalkylsulfonyl includes trifluoromethanesulfonyl, pentafluoroethylsulfonyl, etc. A halogen atom (“halo”) present in compounds, for example in the compounds of formula (I), means a fluorine, chlorine, bromine or iodine atom (“fluoro”, “chloro”, “bromo” or “iodo”), for example fluoro, chloro or bromo.

When the specification states that atom or moiety A is “bonded” or “attached” to atom or moiety B, it means that atom/moiety A is directly bonded to atom/moiety B usually by means of a covalent bond or a double covalent bond, and excludes A being indirectly attached to B via one or more intermediate atoms/moieties (e.g. excludes A-C-B); unless it is clear from the context that another meaning is intended.

When R¹ is C₁₋₄alkyl or C₁₋₃fluoroalkyl, it can be straight-chained or branched. Where R¹ is C₁₋₄alkyl then it can for example be methyl, ethyl, n-propyl, isopropyl or n-butyl. When R¹ is C₁₋₃fluoroalkyl, then R¹ can for example be C₁fluoroalkyl such as monofluoromethyl, difluoromethyl, trifluoromethyl; or R¹ can be C₂fluoroalkyl such as pentafluoroethyl or more preferably C₁fluoroalkyl-CH₂— such as 2,2,2-trifluoroethyl(CF₃CH₂—), 2,2-difluoroethyl(CHF₂CH₂—), or 2-fluoroethyl(CH₂FCH₂—).

Preferably, R¹ is C₁₋₃alkyl (e.g. methyl, ethyl or n-propyl), C₁₋₃fluoroalkyl or —CH₂CH₂OH. R¹ is more preferably C₁₋₃alkyl, C₁₋₂fluoroalkyl, or —CH₂CH₂OH. Still more preferably, R¹ is C₂₋₃alkyl (e.g. ethyl or n-propyl), C₂fluoroalkyl (e.g. C₁fluoroalkyl-CH₂— such as CF₃—CH₂—) or —CH₂CH₂OH; in particular ethyl, n-propyl or —CH₂CH₂OH. Yet more preferably, R¹ is C₂alkyl or C₂fluoroalkyl. R¹ is most preferably ethyl.

Representative examples of R¹ include ethyl.

In another aspect of the invention R² is C₂₋₄alkyl, C₃₋₅cycloalkyl or —CH₂cyclopropyl.

Representative examples of R² include ethyl, propyl, cyclopropyl, cyclobutyl, cyclopentyl and cyclopropylmethyl.

Preferably, in R³ there is one substituent or no substituent.

In one embodiment, R³ is the optionally substituted C₃₋₈cycloalkyl or the optionally substituted heterocyclic group of sub-formula (aa), (bb) or (cc).

In one optional embodiment, when R³ is optionally substituted C₃₋₈cycloalkyl, it is not unsubstituted C₅cycloalkyl, i.e. not unsubstituted cyclopentyl. In this case, more suitably, R³ is optionally substituted C₆₋₈cycloalkyl.

When R³ is optionally substituted C₃₋₈cycloalkyl, it is more preferably optionally substituted C₆₋₇cycloalkyl or optionally substituted C₆cycloalkyl (i.e. cyclohexyl).

Suitably, when R³ is optionally substituted C₃₋₈cycloalkyl, then R³ is C₃₋₈cycloalkyl (e.g. C₆₋₇cycloalkyl) optionally substituted with one or two substituents independently being oxo (═O); OH; C₁alkoxy; C₁fluoroalkoxy (e.g. trifluoromethoxy or difluoromethoxy); NHR²¹ wherein R²¹ is a hydrogen atom (H) or C₁₋₂alkyl (more preferably R²¹ is H); C₁₋₂alkyl such as methyl; C₁fluoroalkyl such as —CH₂F or —CHF₂; —CH₂OH; —CH₂NHR²² wherein R²² is H; —C(O)OR²³ wherein R²³ is H or methyl; —C(O)NHR²⁴ wherein R²⁴ is H or methyl; —C(O)R²⁵ wherein R²⁵ is methyl; fluoro; hydroxyimino (═N—OH); or (C₁₋₄alkoxy)imino(—N—OR²⁶ where R²⁶ is C₁₋₄alkyl); and wherein any OH, alkoxy, fluoroalkoxy or NHR²¹ substituent is not substituted at the R³ ring carbon attached (bonded) to the —NH— group of formula (I) and is not substituted at either R³ ring carbon bonded to the Y group of the heterocyclic group (aa), (bb) or (cc).

Preferably, when R³ is optionally substituted C₃₋₈cycloalkyl, then R³ is C₃₋₈cycloalkyl (e.g. C₆₋₇cycloalkyl) optionally substituted with one or two substituents independently being oxo (═O); OH; NHR²¹ wherein R²¹ is a hydrogen atom (H); C₁₋₂alkyl such as methyl; C₁fluoroalkyl such as —CH₂F or —CHF₂; —C(O)OR²³ wherein R²³ is H or methyl; —C(O)NHR²⁴ wherein R²⁴ is H or methyl; fluoro; hydroxyimino (═N—OH); or (C₁₋₂alkoxy)imino (═N—OR²⁶ where R²⁶ is C₁₋₂alkyl).

More preferably, when R³ is optionally substituted C₃₋₈cycloalkyl, then R³ is C₃₋₈cycloalkyl (e.g. C₆₋₇cycloalkyl) optionally substituted with one or two substituents independently being oxo (═O); OH; NHR²¹ wherein R²¹ is a hydrogen atom (H); methyl; —CH₂F; —CHF₂; —C(O)OR²³ wherein R²³ is H; —C(O)NHR²⁴ wherein R²⁴ is H or methyl (preferably H); fluoro; hydroxyimino (═N—OH); or methoxyimino (═N—OR²⁶ where R²⁶ is methyl).

Still more preferably, when R³ is optionally substituted C₃₋₈cycloalkyl, then R³ is C₃₋₈cycloalkyl (e.g. C₆₋₇cycloalkyl) optionally substituted with one or two substituents independently being oxo (═O); OH; methyl; —C(O)NHR²⁴ wherein R²⁴ is H; fluoro; hydroxyimino (═N—OH); or methoxyimino (═N—OR²⁶ where R²⁶ is methyl).

Yet more preferably, when R³ is optionally substituted C₃₋₈cycloalkyl, then R³ is C₃₋₈cycloalkyl (e.g. C₆₋₇cycloalkyl) optionally substituted with one or two substituents independently being OH; —C(O)NHR²⁴ wherein R²⁴ is H; oxo (═O) or hydroxyimino (═N—OH).

In one optional embodiment, in R³, the C₃₋₈cycloalkyl can be unsubstituted.

When R³ is optionally substituted C₃₋₈cycloalkyl or optionally substituted C₅₋₇cycloalkenyl, e.g. optionally substituted C₅₋₈cycloalkyl or C₅₋₇cycloalkyl, such as optionally substituted C₆cycloalkyl (optionally substituted cyclohexyl) or optionally substituted cyclohexenyl, the one or two optional substituents if present suitably can comprise a substituent (for example is or are substituent(s)) at the 3-, 4- and/or 5-position(s), e.g. at the 3- and/or 4-position(s), of the R³ cycloalkyl or cycloalkenyl ring. (In this connection and generally herein, the l-position of the R³ ring, e.g. of the R³ cycloalkyl or cycloalkenyl ring is deemed to be the connection point to the —NH— in formula (I)=the ring atom connecting to the —NH— in formula (I)).

Suitably, for R³, and in particular when R³ is optionally substituted C₃₋₈cycloalkyl or optionally substituted C₅₋₇cycloalkenyl, R³ is not substituted (other than optionally by alkyl or fluoroalkyl) at the ring atom connecting to the —NH— in formula (I), and R³ is not substituted (other than optionally by alkyl, fluoroalkyl or NHR²¹) at the two ring atoms either side of (bonded to) the connecting atom. For example, suitably, for R³, and in particular when R³ is optionally substituted C₃₋₈cycloalkyl or optionally substituted C₅₋₇cycloalkenyl, R³ is not substituted at the ring atom connecting to the —NH— in formula (I), and R³ is not substituted at the two ring atoms either side of (bonded to) the connecting atom.

Suitably, for R³, and in particular when R³ is optionally substituted C₃₋₈cycloalkyl or optionally substituted C₅₋₇cycloalkenyl, the one or two optional R³ substituents if present can comprise a substituent (for example is or are substituent(s)):
(a) at the 3-position of a R³ cyclobutyl ring, or
(b) at the 3- and/or 4-position(s) of a R³ cyclopentyl or cyclopentenyl ring, or
(c) at the 3-, 4- and/or 5-position(s) of a R³ cyclohexyl or cyclohexenyl ring, or
(d) at the 3-, 4-, 5- and/or 6-position(s) of a R³ cycloheptyl or cycloheptenyl ring, or
(e) at the 3-, 4-, 5-, 6- and/or 7-position(s) of a R³ cyclooctyl ring, and/or
(f) at the 1-, 2- and/or highest-numbered-position(s) of a R³ cycloalkyl or cycloalkenyl ring, for alkyl or fluoroalkyl substituent(s), and/or
(g) at the 2- and/or highest-numbered-position(s) of a R³ cycloalkyl or cycloalkenyl ring, for NHR²¹ or fluoro substituent(s).

When R³ is optionally substituted C₃₋₈cycloalkyl, any OH, alkoxy, fluoroalkoxy, —CH₂CH₂OH or —CH₂NHR²² substituent (particularly any OH substituent) is suitably at the 3-, 4- or 5-position, e.g. 3- or 5-position, of the R³ cycloalkyl (e.g. C₆₋₈cycloalkyl) ring. Optionally, any OH, alkoxy, fluoroalkoxy, —CH₂CH₂OH or —CH₂NHR²² substituent (particularly any OH substituent) can be: at the 3-position of a R³ cyclobutyl ring; or at the 3- or 4-position of a R³ C₅cycloalkyl (cyclopentyl) ring; or at the 3-, 4- or 5-position, of a R³ C₆cycloalkyl (cyclohexyl) ring e.g. at the 3- or 5-position of a R³ cyclohexyl ring especially for any OH substituent; or at the 3-, 4-, 5- or 6-position of a R³ cycloheptyl ring, or at the 3-, 4-, 5-, 6- or 7-position of a R³ cyclooctyl ring. Suitably, any OH, alkoxy, fluoroalkoxy, —CH₂CH₂OH or —CH₂NHR²² substituent (particularly any OH substituent) is at the 3- or 4-position of a R³ C₅cycloalkyl (cyclopentyl) ring; or more suitably at the 3-, 4- or 5-position, still more suitably at the 3- or 5-position, of a R³ C₆cycloalkyl(cyclohexyl) ring.

Suitably, when R³ is optionally substituted C₃₋₈cycloalkyl or optionally substituted C₅₋₇cycloalkenyl, any —C(O)OR²³, —C(O)NHR²⁴, —C(O)R²⁵, —CH₂OH, or fluoro substituent is: at the 3-position of a R³ cyclobutyl ring; or at the 3- or 4-position of a R³ C₅cycloalkyl(cyclopentyl) or cyclopentenyl ring; or at the 3-, 4- or 5-position, preferably at the 4-position, of a R³ C₆cycloalkyl(cyclohexyl) or cyclohexenyl ring; or at the 3-, 4-, 5- or 6-position of a R³ cycloheptyl or cycloheptenyl ring, or at the 3-, 4-, 5-, 6- or 7-position of a R³ cyclooctyl ring. Any —C(O)OR²³, —C(O)NHR²⁴, —C(O)R²⁵, —CH₂OH, or fluoro substituent, e.g. any —C(O)NHR²⁴ or fluoro substituent, is suitably at the 3-, 4- or 5-position, more suitably at the 4-position, of a R³C₆cycloalkyl(cyclohexyl) or cyclohexenyl ring. It is particularly preferable for any —C(O)NHR²⁴ substituent to be at the 4-position of a R³ cyclohexyl ring.

When R³ is optionally substituted C₃₋₈cycloalkyl, any NHR²¹ substituent is at any position other than the 1-position (the ring atom connecting to the —NH— in formula (I)), e.g. at the 3-, 4-, 5-, 6-, 7- or 8-position. Preferably, any NHR²¹ substituent is at the 2-, 3-, 5- or 6-position, or more preferably at the 3- or 5-position, of a R³ cyclohexyl ring.

When R³ is optionally substituted C₃₋₈cycloalkyl or optionally substituted C₅₋₇cycloalkenyl, any alkyl or fluoroalkyl substituent can for example be at the 1-, 2-, 3-, 4-, 5-, 6-, 7- or 8-position, for example at the 1-, 2-, 3-, 5- or 6-position, e.g. the 1-position, of the R³ ring. Preferably, any alkyl or fluoroalkyl substituent is at the 1-, 2-, 3-, 5- or 6-position, or more preferably at the 1-, 3- or 5-position, of a R³ cyclohexyl or cyclohexenyl ring.

When R³ is optionally substituted C₃₋₈cycloalkyl, any oxo (═O), hydroxyimino (═N—OH); or (C₁₋₄alkoxy)imino (═N—OR²⁶) substituent is suitably at the 3-, 4- or 5-position, e.g. at the 4-position, of the R³ cycloalkyl (e.g. C₆₋₈cycloalkyl e.g. cyclohexyl) ring. Preferably any such substituent is at the 4-position of a R³ cyclohexyl ring.

When R³ is optionally substituted C₃₋₈cycloalkyl (e.g. C₆₋₇cycloalkyl), R³ is preferably cyclohexyl (i.e. unsubstituted); or cycloheptyl (i.e. unsubstituted); or cyclohexyl substituted by one substituent being oxo (═O), OH, NHR²¹, C₁₋₂alkyl, C₁₋₂fluoroalkyl, —CH₂OH, —C(O)OR²³, —C(O)NHR²⁴, —C(O)R²⁵, fluoro, hydroxyimino (═N—OH), or (C₁₋₄alkoxy)imino (═N—OR²⁶); or cyclohexyl substituted by two fluoro substituents. More preferably, R³ is cyclohexyl (i.e. unsubstituted); or cycloheptyl (i.e. unsubstituted); or cyclohexyl substituted by one substituent being oxo (═O), OH, NHR²¹, C₁₋₂alkyl, C₁₋₂fluoroalkyl, —C(O)OR²³, —C(O)NHR²⁴, fluoro, hydroxyimino (═N—OH), or (C₁₋₂alkoxy)imino (═N—OR²⁶ wherein R²⁶ is C₁₋₂alkyl); or cyclohexyl substituted by two fluoro substituents. Still more preferably R³ is cyclohexyl (i.e. unsubstituted) or cyclohexyl substituted by one oxo (═O), hydroxyimino (═N—OH), —C(O)NH₂, methyl or OH substituent. The optional substituent can for example be at the 3- or 4-position, of the R³ cyclohexyl ring. Preferably, any OH substituent is preferably at the 3-position of a R³ cyclohexyl ring, and/or any oxo (═O), hydroxyimino (═N—OH), (C₁₋₄alkoxy)imino (═N—OR²⁶) or —C(O)NH₂ substituent is preferably at the 4-position of a R³ cyclohexyl ring, and/or any alkyl or fluoroalkyl substituent is preferably at the 1-, 3- or 5-position of a R³ cyclohexyl ring.

When R³ is optionally substituted C₆₋₇cycloalkyl, R³ can for example be 4-hydroxy-cyclohexyl (i.e. 4-hydroxycyclohexan-1-yl), 4-methylcyclohexyl, 3-fluorocyclohexyl, 2-aminocyclohexyl, 3-(HO(O)C)cyclohexyl or 3-oxocyclohexyl, but R³ is more preferably cyclohexyl (i.e. unsubstituted), cycloheptyl (i.e. unsubstituted), 3-hydroxy-cyclohexyl (i.e. 3-hydroxycyclohexan-1-yl) (e.g. in a cis configuration), 4-oxo-cyclohexyl (i.e. 4-oxocyclohexan-1-yl), 4-(hydroxyimino)cyclohexyl (i.e. 4-(hydroxyimino)cyclohexan-1-yl), 4-(C₁₋₂alkoxyimino)cyclohexyl, 4-(aminocarbonyl)cyclohexyl (i.e. 4-(aminocarbonyl)cyclohexan-1-yl) (e.g. in a cis configuration), 1-methylcyclohexyl, 3-methylcyclohexyl, 4,4-(difluoro)cyclohexyl, or 3-aminocyclohexyl.

When R³ is optionally substituted C₆₋₇cycloalkyl, R³ is most preferably cyclohexyl (i.e. unsubstituted), 3-hydroxy-cyclohexyl (i.e. 3-hydroxycyclohexan-1-yl) (e.g. in a cis configuration), 4-oxo-cyclohexyl (i.e. 4-oxocyclohexan-1-yl), 4-(hydroxyimino)cyclohexyl (i.e. 4-(hydroxyimino)cyclohexan-1-yl), or 4-(aminocarbonyl)cyclohexyl (i.e. 4-(aminocarbonyl)cyclohexan-1-yl) (e.g. in a cis configuration).

When R³ is optionally substituted C₅cycloalkyl (optionally substituted cyclopentyl), R³ can for example be cyclopentyl (i.e. unsubstituted) or more suitably 3-hydroxy-cyclopentyl.

When R³ is optionally substituted mono-unsaturated-C₅₋₇cycloalkenyl, preferably it is optionally substituted mono-unsaturated-C₅₋₆cycloalkenyl, more preferably optionally substituted mono-unsaturated-C₆cycloalkenyl (i.e. optionally substituted mono-unsaturated-cyclohexenyl=optionally substituted cyclohexenyl). For example, the R³ cyclohexenyl can be optionally substituted cyclohex-3-en-1-yl.

When R³ is optionally substituted mono-unsaturated-C₅₋₇cycloalkenyl, in one optional embodiment the R³ cycloalkenyl is optionally substituted with one or two substituents independently being fluoro or methyl. Preferably, in this embodiment, if there are two substituents then they are not both methyl.

In another optional embodiment, the R³ cycloalkenyl (e.g. cyclohexenyl) is optionally substituted with one substituent being fluoro or C₁₋₂alkyl (preferably fluoro or methyl); more preferably the R³ cycloalkenyl (e.g. cyclohexenyl) is substituted with one fluoro substituent or is unsubstituted. For example, the R³ optionally substituted cycloalkenyl can be cyclohex-3-en-1-yl (i.e. unsubstituted) or 4-fluoro-cyclohex-3-en-1-yl.

For R³ cycloalkenyl, the optional substituent(s) can for example be at the 1-, 2-, 3-, 4-, 5- or 6-position(s) of the cycloalkenyl ring.

When R³ is the heterocyclic group of sub-formula (aa), (bb) or (cc), then Y is preferably O or NR¹⁰, most preferably O.

Suitably, R¹⁰ is a hydrogen atom (H), methyl, ethyl, C(O)NH₂, C(O)—C₁₋₂alkyl or C(O)—C₁fluoroalkyl. Preferably, R¹⁰ is not C₁₋₂alkyl or C₁₋₂fluoroalkyl. Suitably R¹⁰ is not CH₂C(O)NH₂.

More preferably, R¹⁰ is a hydrogen atom (H), C(O)NH₂, C(O)—C₁₋₂alkyl (e.g. C(O)methyl) or C(O)—C₁fluoroalkyl (e.g. C(O)—CF₃). Still more preferably R¹⁰ is H, C(O)NH₂ or C(O)methyl; for example C(O)NH₂.

When R³ is the heterocyclic group of sub-formula (aa), (bb) or (cc), then it is preferable that R³ is the heterocyclic group of sub-formula (aa) or (bb), more preferably of sub-formula (bb).

In sub-formula (bb), n¹ is preferably 1. In sub-formula (cc), n² is preferably 1. That is, six-membered rings are preferred in the R³ heterocyclic group.

Suitably, in R³, the heterocyclic group of sub-formula (aa), (bb) or (cc) is unsubstituted on a ring carbon. (In this connection, where Y is NR¹⁰, R¹⁰ is not a substituent on a ring carbon).

In the R³ heterocyclic group of sub-formula (aa), (bb) or (cc), the one or two optional substituents preferably comprise (e.g. is or independently are) OH; oxo (═O); C₁₋₂alkyl (e.g. methyl) or C₁₋₂fluoroalkyl (e.g. C₁fluoroalkyl such as —CH₂F or —CHF₂). More preferably, in the R³ heterocyclic group of sub-formula (aa), (bb) or (cc), the one or two optional substituents comprise (e.g. is or independently are) C₁₋₂alkyl (e.g. methyl) or oxo; most preferably the one or two optional substituents comprise (e.g. is or are) oxo (═O).

In the R³ heterocyclic group of sub-formula (aa), (bb) or (cc), any oxo (═O) substituent is preferably on a carbon atom bonded (adjacent) to Y, e.g. is on a carbon atom bonded (adjacent) to Y only when Y is O or NR¹⁰.

In the R³ heterocyclic group of sub-formula (aa), (bb) or (cc), any oxo (═O) substituent can suitably be at the 2-, 3-, 4-, 5- or 6-position of the R³ heterocyclic ring. For example any oxo (═O) substituent(s) can be: at the 2-, 4- or 5-position(s) (e.g. 2-position or 4-position, or two oxo substituents at 2- and 4-positions) of a R³ heterocyclic group of sub-formula (aa), at the 2-, 4-, 5- or 6-position(s) (e.g. 4-position) of a six-membered R³ heterocyclic group of sub-formula (cc) wherein n² is 1, at the 2-, 3-, 5-, 6- or 7-position(s) (e.g. 5-position) of a seven-membered R³ heterocyclic group of sub-formula (bb) wherein n¹ is 2, or at the 2-, 4-, 5-, 6- or 7-position(s) (e.g. 2-position) of a seven-membered R³ heterocyclic group of sub-formula (cc) wherein n² is 2.

(In this connection and generally herein, the 1-position of the R³ heterocyclic ring is deemed to be the connection point to the —NH— in formula (I)=the ring atom connecting to the —NH— in formula (I), and the remaining positions of the ring are then numbered so that the ring heteroatom takes the lowest possible number).

In the R³ heterocyclic group of sub-formula (aa), (bb) or (cc), any alkyl or fluoroalkyl substituent can for example be at the 1-, 2-, 3-, 4-, 5- or 6-position, e.g. the 1-position, of the R³ heterocyclic ring, for example at the 1-, 3- or 5-position of a six-membered R³ heterocyclic ring.

In the R³ heterocyclic group of sub-formula (aa), (bb) or (cc), any OH substituent can be: at the 5-position of a six-membered R³ heterocyclic group of sub-formula (cc) wherein n² is 1; at the 5- or 6-position of a seven-membered R³ heterocyclic group of sub-formula (cc) wherein n² is 2; or at the 6-position of a seven-membered R³ heterocyclic group of sub-formula (bb) wherein n¹ is 2.

Any other substituents of the R³ heterocyclic group can optionally be positioned on the R³ heterocyclic ring at numerical positions as described herein for when R³ is optionally substituted C₅₋₇cycloalkyl, all necessary changes to the wording being made.

In the R³ heterocyclic group of sub-formula (aa), (bb) or (cc), preferably, only C₁₋₂alkyl, C₁₋₂fluoroalkyl, fluoro or oxo (═O) substitution or no substitution is allowed independently at each of the 2- and highest-numbered-positions of the R³ heterocyclic ring (e.g. at each of the 2- and 6-positions of a six-membered R³ heterocyclic ring), and/or only C₁₋₂alkyl, C₁₋₂fluoroalkyl or fluoro substitution or no substitution is allowed at the 1-position of the R³ heterocyclic ring.

When R³ is the heterocyclic group of sub-formula (aa) and Y is NR¹⁰, then R¹⁰ is not C(O)—C₁₋₂alkyl, C(O)—C₁fluoroalkyl or —C(O)—CH₂O—C₁₋₂alkyl. According to one optional embodiment when R³ is the heterocyclic group of sub-formula (aa) and Y is NR¹⁰ then R¹⁰ is optionally not C(O)NHMe, C(O)—C₁₋₂alkyl, C(O)—C₁fluoroalkyl or —C(O)—CH₂O—C₁₋₂alkyl.

In one preferable embodiment, Y is O, S, SO₂ or NH when R³ is the heterocyclic group of sub-formula (aa).

When R³ is the heterocyclic group of sub-formula (bb), n¹ is 1, and Y is NR¹⁰ (e.g. when NHR³ is 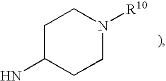
then R¹⁰ is not C₁₋₂alkyl or C₁₋₂fluoroalkyl. More preferably, when R³ is the heterocyclic group of sub-formula (bb) wherein n¹ is 1 or 2 and Y is NR¹⁰, then R¹⁰ is preferably not C₁₋₂alkyl or C₁₋₂fluoroalkyl.

In one embodiment, when R³ is the heterocyclic group of sub-formula (bb), then preferably Y is O, S, SO₂ or NR¹⁰ wherein R¹⁰ is H, C(O)NH₂, C(O)—C₁₋₂alkyl (e.g. C(O)methyl) or C(O)—C₁fluoroalkyl (e.g. C(O)—CF₃), or more preferably R¹⁰ is H, C(O)NH₂ or C(O)Me, for example C(O)NH₂ or C(O)Me most preferably CONH₂.

In one optional embodiment, when R³ is the heterocyclic group of sub-formula (cc), then optionally Y is O, S, SO₂ or NR¹⁰ wherein R¹⁰ is H, C(O)NH₂, C(O)—C₁₋₂alkyl (e.g. C(O)methyl) or C(O)—C₁fluoroalkyl (e.g. C(O)—CF₃). In this case R¹⁰ can for example be H, C(O)NH₂ or C(O)Me, for example H.

Optionally, for sub-formula (bb) and/or for sub-formula (cc), Y is O or NR¹⁰.

When R³ is optionally substituted C₃₋₈cycloalkyl (e.g. C₆₋₇cycloalkyl) or optionally substituted mono-unsaturated-C₅₋₇cycloalkenyl or an optionally substituted heterocyclic group of sub-formula (aa), (bb) or (cc), then a substituent can be in the cis or trans configuration with respect to the —NH— group of formula (I) to which R³ is attached (bonded); this includes mixtures of configurations wherein the stated configuration is the major component. For example, an OH or —C(O)NHR²⁴ substituent on C₆₋₇cycloalkyl can for example be in the cis configuration and/or a NHR²¹ substituent on C₆₋₇cycloalkyl can for example be in the cis or trans configuration, with respect to the —NH— group of formula (I) to which R³ is attached (bonded), including mixtures of configurations wherein the stated configuration is the major component.

When R³ is a bicyclic group of sub-formula (ee), then preferably Y¹, Y² and Y³ are all CH₂.

Preferably, NHR³ is of sub-formula (a), (a1), (b), (c), (c 1), (c 2), (c 3), (c 4), (c 5), (c 6), (c 7), (d), (e), (f), (g), (g1), (g2), (g3), (g4), (h), (i), (j), (k), (k1), (k2), (L), (m), (m1), (m2), (m3), (m5), (n), (o), (o1), (o2), (o3), (p), (p1), (p2), (p3), (p4), (p5), (p6), (p7), (p8), (p9), (p10), (p11) or (q):

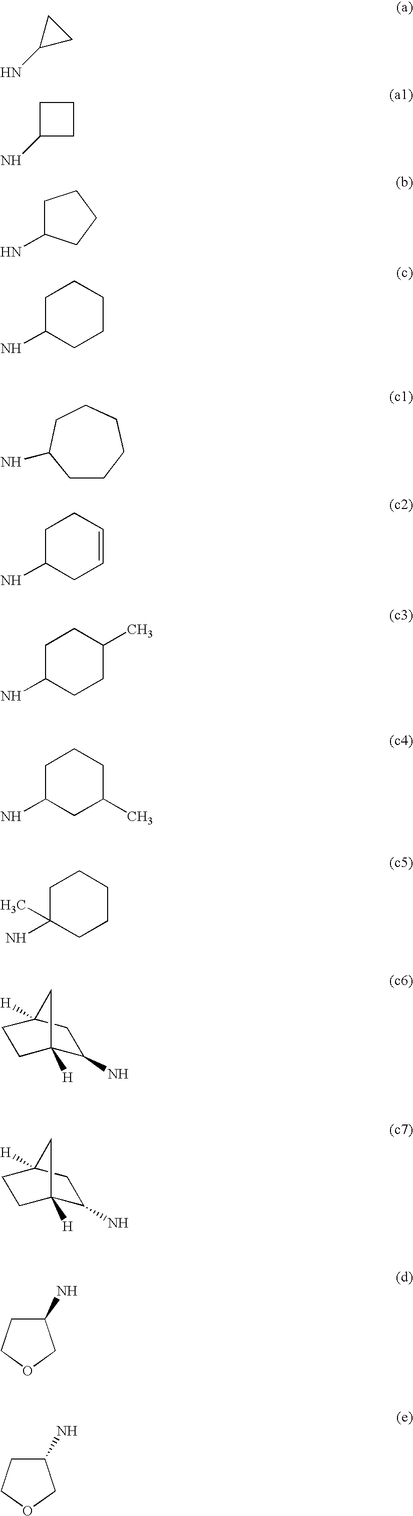

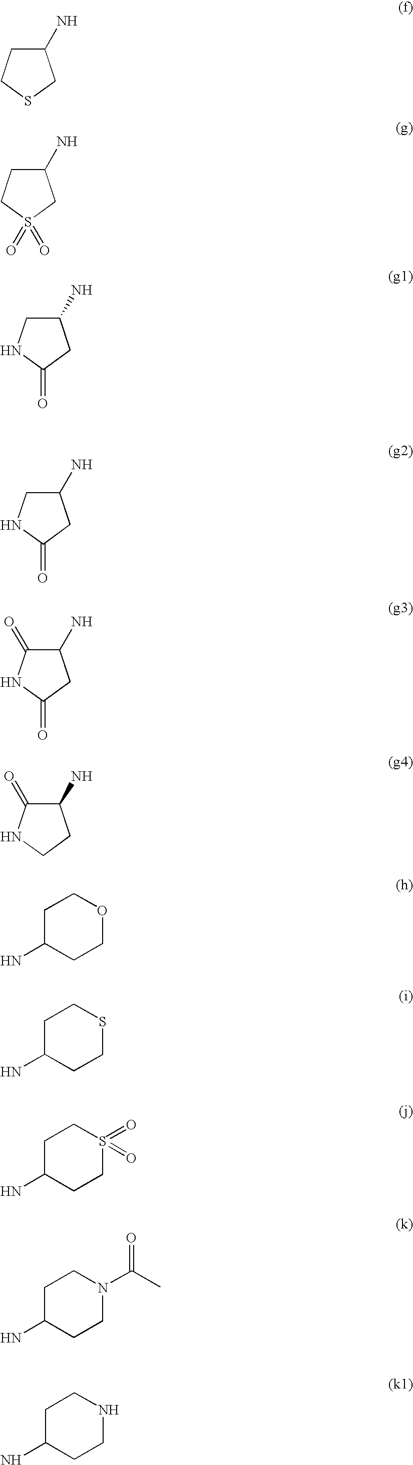

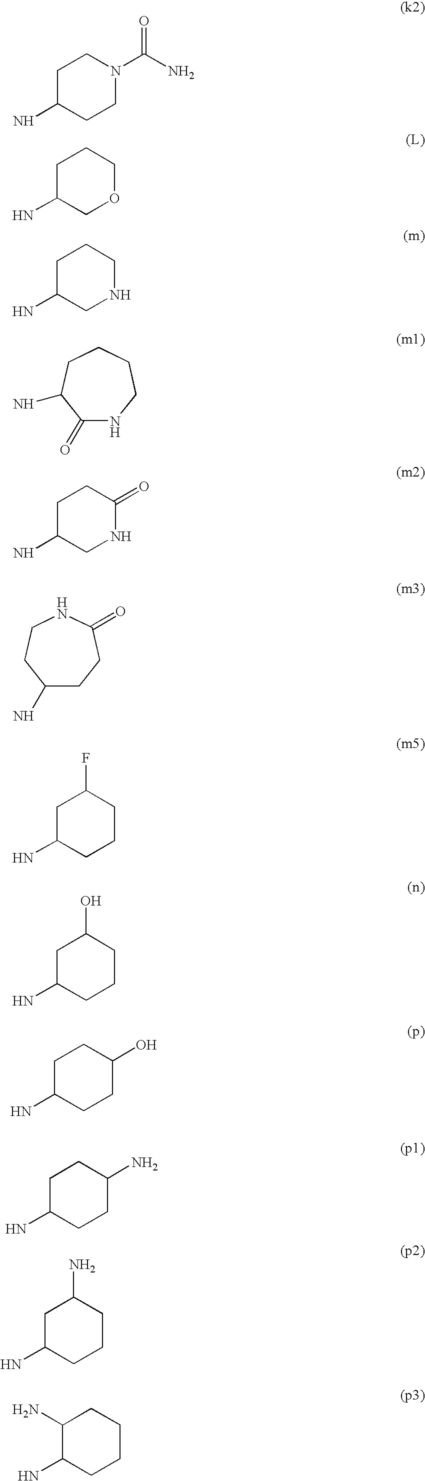

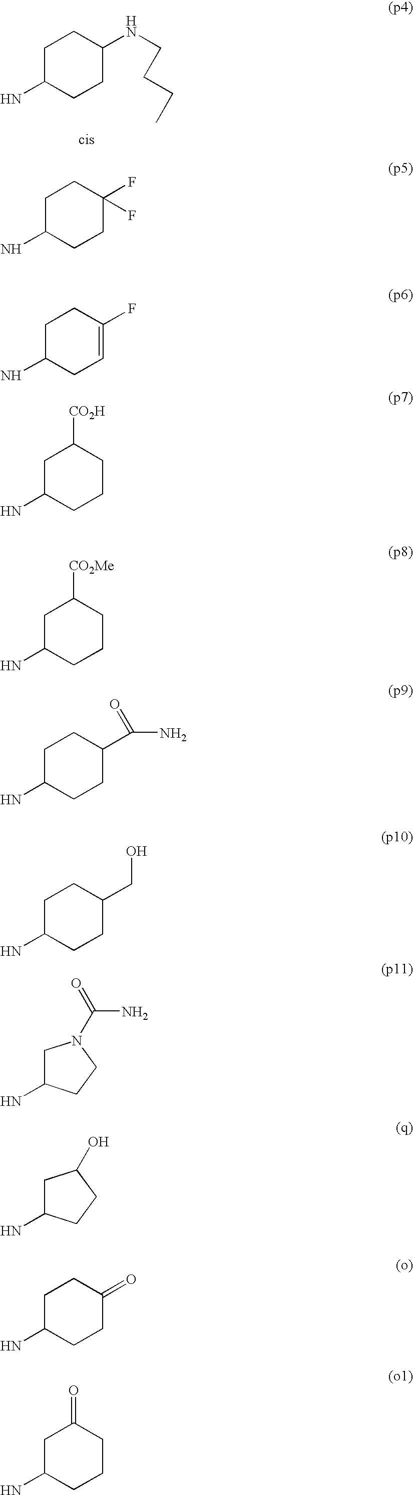

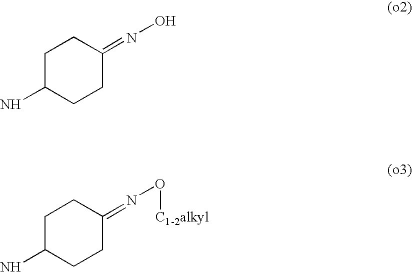

In the sub-formulae (a) to (q) etc above, the —NH— connection point of the NHR³ group to the 4-position of the pyrazolopyridine of formula (I) is underlined.

Preferably, NHR³ is of sub-formula (c), (c1), (c 2), (c 3), (c 4), (c 5), (c 6), (c 7), (d), (e), (f), (g1), (g4), (h), (i), (j), (k), (k1), (k2), (L), (m), (m1), (m2), (m3), (m5), (n), (o), (o1), (o2), (o3), (p), (p2), (p5), (p6), (p7), (p9), (p10), (p11) or (q). More preferably, NHR³ is of sub-formula (c), (c1), (c 4), (c 5), (h), (i), (j), (k), (k2), (m1), (m2), (n), (o), (o2), (o3), (p2), (p5), (16), (p9), (p11) or (q). NHR³ can for example be of sub-formula (c), (p11), (h), (k), (k2), (n), (o), (o2) or (p9); or still more preferably (c), (p11), (h), (k2), (n), (o), (o2) or (p9). Most preferably, R³ is tetrahydro-2H-pyran-4-yl or 1-(aminocarbonyl)-4-piperidinyl; that is NHR³ is most preferably of sub-formula (h) or (k2), as shown above.

In another aspect of the invention R³ is C₃₋₈cycloalkyl (e.g. C₆₋₇cycloalkyl) optionally substituted with one or two substituents independently being oxo (═O); OH; methyl; —C(O)NHR²⁴ wherein R²⁴ is H; fluoro; hydroxyimino (—N—OH); or methoxyimino(═N—OR²⁶ where R²⁶ is methyl).

In another aspect of the invention R³ is a 4-(aminocarbonyl)cyclohexyl (i.e. 4-(aminocarbonyl)cyclohexan-1-yl) group of sub-formula (aa), or an N-aminocarbonyl-piperidinyl or an N-aminocarbonyl-pyrrolidinyl or a tetrahydropyranyl group of sub-formula (bb); 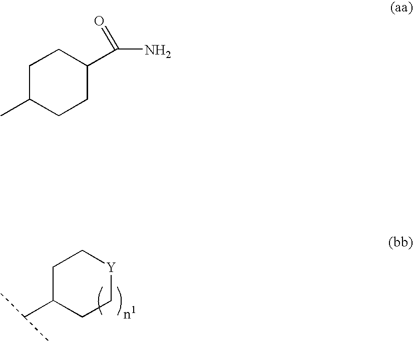
wherein Y is O or NCONH₂ and n¹ is 0 or 1;

and wherein the cyclohexyl group of sub-formula (aa) or the piperidinyl, pyrrolidinyl or tetrahydropyranyl groups of sub-formula (bb) may be further optionally substituted with one or two substituents independently selected from C₁₋₂alkyl; C₁₋₂fluoroalkyl; CH₂OH; —C(O)OR²³ wherein R²³ is H or C₁₋₂alkyl; —C(O)NHR²³; or fluoro; on any ring carbon; as well as, on the C2, C3, C5 and C6 of the cyclohexyl group of (aa), the C2 or C6 of the piperidinyl ring or the C5 of the pyrrolidinyl ring of (bb), a substituent selected from OH; C₁₋₂alkoxy; C₁₋₂fluoroalkoxy; or alkoxy.

Representative examples of R³ include tetrahydro-2H-pyran-4-yl.

When NHR³ is of sub-formula (n), then preferably it is in the cis configuration, i.e. preferably it is a cis-(3-hydroxycyclohexan-1-yl)amino group, e.g. in any enantiomeric form or mixture of forms such as a racemic mixture.

When NHR³ is of sub-formula (p9), then preferably it is in the cis configuration, i.e. preferably it is a cis-[4-(aminocarbonyl)cyclohexan-1-yl]amino group.

Where R⁴ is C₁₋₂fluoroalkyl, then it can be C₁fluoroalkyl such as monofluoromethyl, difluoromethyl or trifluoromethyl.

R^{4a} can suitably be a hydrogen atom (H) or methyl (Me), more suitably H.

R⁴ can for example be a hydrogen atom (H); methyl, ethyl, C₁fluoroalkyl, —CH₂OH, —CH(Me)OH, —CH₂CH₂OH, or −CH₂OMe; or preferably a hydrogen atom (H), methyl, ethyl, —CH₂OH, or —CH₂OMe. More preferably, R⁴ is H, methyl, ethyl, —CH₂OH, or —CH₂OMe.

Representative examples of R⁴ include H, methyl and ethyl.

In another aspect of the invention, R⁵ is a hydrogen atom (H), methyl, ethyl, n-propyl, or iso-propyl.

Representative examples of R⁵ include H.

In another aspect of the invention, in sub-formulae (y) and (y1) m is 1, A is C—R^{6A}, B is C—R^{6B}, D is C—R^{6D} and E is C—R^{6E}.

Representative examples of (y) include 2,3-dihydro-1H-inden-2-yl.

It is preferable that Ar has the sub-formula (x).

Preferably, in sub-formula (x), two or more (more preferably three or more) of A, B, D, E and F are independently C—H (carbon-hydrogen), C—F (carbon-fluorine) or nitrogen (N).

Preferably, in sub-formula (x), three or more of A, B, D, E and F are independently C—H (carbon-hydrogen), C—F (carbon-fluorine), nitrogen (N), or nitrogen-oxide (N⁺—O⁻)).

Preferably, in sub-formula (x), two or more (e.g. three or more) of A, B, D, E and F are independently C—H (carbon-hydrogen), C—F (carbon-fluorine), or nitrogen (N); and one or more (e.g. two or more) others of A, B, D, E and F are independently C—H (carbon-hydrogen), C—F (carbon-fluorine), C—Cl (carbon-chlorine), C-Me, C—OMe, or nitrogen (N). More preferably, in sub-formula (x), two or more (e.g. three or more) of A, B, D, E and F are C—H (carbon-hydrogen); and one or more (e.g. two or more) others of A, B, D, E and F are independently C—H (carbon-hydrogen), C—F (carbon-fluorine), C—Cl (carbon-chlorine), C-Me, C—OMe, or nitrogen (N).

Preferably, in sub-formula (x), two or more (e.g. three or more, e.g. four or more) of A, B, D, E and F are C—H.

Preferably, in sub-formula (x), no more than one (more preferably none) of A, B, D, E and F are independently nitrogen or nitrogen-oxide (N⁺—O⁻)).

Preferably, in sub-formula (x), none of A, B, D, E and F are nitrogen-oxide (N⁺—O⁻)).

Preferably, Ar has the sub-formula (x) which is sub-formula (x1), (x2), (x3), (x4), (x5), (x6), (x7), (x8), (x9), (x10), (x11), (x12), (x13), (x14), (x15) or (x16):

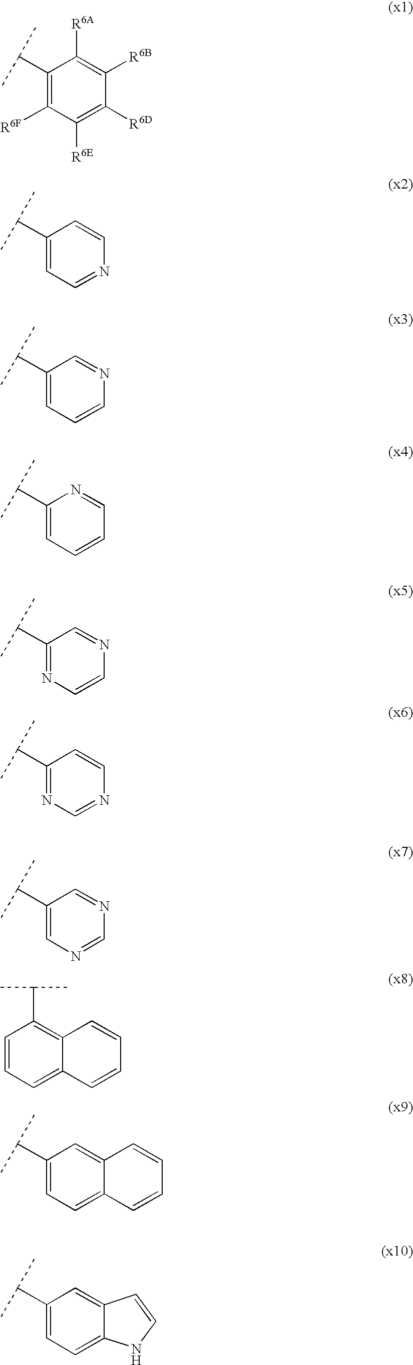

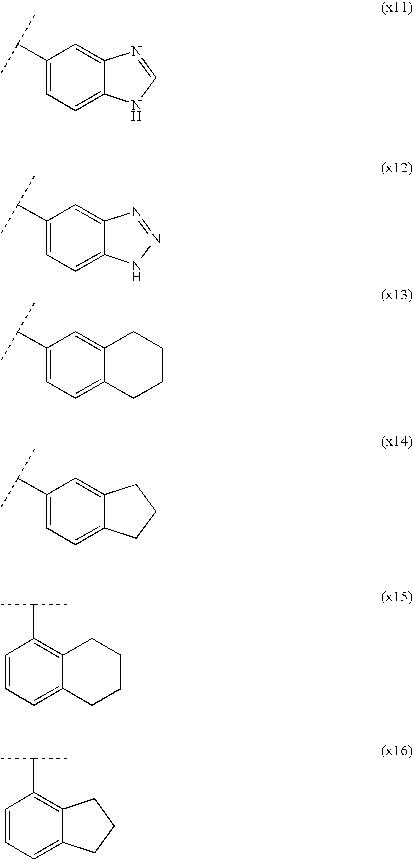

More preferably, Ar has the sub-formula (x) which is sub-formula (x1), (x2), (x3), (x8), (x13), or (x14). Still more preferably, Ar has the sub-formula (x) which is sub-formula (x1), (x8), (x13), or (x14). Most preferably, Ar has the sub-formula (x) which is sub-formula (x1).

In sub-formula (x), preferably, R^{6A}, R^{6B}, R^{6D}, R^{6E} and/or R^{6F}, independently of each other, is or are: a hydrogen atom (H), a fluorine, chlorine, bromine or iodine atom, methyl, ethyl, n-propyl, isopropyl, C₄alkyl, trifluoromethyl, —CH₂OH, methoxy, ethoxy, n-propoxy, isopropoxy, C₁fluoroalkoxy (e.g. trifluoromethoxy or difluoromethoxy), cyclohexyloxy; cyclopentyloxy; nitro (—NO₂), OH, C₁₋₃alkylS(O)₂— (such as MeS(O)₂—), C₁₋₃alkylS(O)₂—NH— such as Me-S(O)₂—NH—, Me₂N—S(O)₂—, H₂N—S(O)₂—, —CONH₂, —CONHMe, —C(O)OH, cyano (—CN), NMe₂, or C₁₋₂alkyl-S(O)₂—CH₂— such as Me-S(O)₂—CH₂—.

More preferably, R^{6A}, R^{6B}, R^{6D}, R^{6E} and/or R^{6F}, independently of each other, is or are: a hydrogen atom (H), a fluorine, chlorine, bromine or iodine atom, methyl, ethyl, n-propyl, isopropyl, isobutyl, trifluoromethyl, —CH₂OH, methoxy, ethoxy, n-propoxy, isopropoxy, C₁fluoroalkoxy (e.g. trifluoromethoxy or difluoromethoxy), nitro (—NO₂), OH, C₁₋₃alkylS(O)₂— such as MeS(O)₂—, C₁₋₂alkylS(O)₂—NH— such as Me-S(O)₂—NH—, —CONH₂, cyano (—CN), or C₁₋₂alkylS(O)₂—CH₂— such as Me-S(O)₂—CH₂.

Still more preferably, R^{6A}, R^{6B}, R^{6D}, R^{6E} and/or R^{6F}, independently of each other, is or are: a hydrogen atom (H), a fluorine, chlorine or bromine atom, methyl, ethyl, n-propyl, isopropyl, trifluoromethyl, —CH₂OH, methoxy, ethoxy, n-propoxy, difluoromethoxy, OH or MeS(O)₂—.

When two adjacent groups selected from R^{6A}, R^{6B}, R^{6D}, R^{6E} and R^{6F} are taken together, then, preferably, when taken together they are: —CH═CH—CH═CH₂—, —(CH₂)ₙ^{14a}— where n^{14a} is 3, 4 or 5 (e.g. 3 or 4), —O—(CMe₂)-O—, —O—(CH₂)ₙ^{14b}—O— where n^{14b} is 1 or 2; —CH═CH—NR^{15b}—; —N═CH—NR^{15b}—; —N═N—NR^{15b} wherein R^{15b} is H or C₁₋₂alkyl (preferably R^{15b} is H). More preferably, in this embodiment, two adjacent groups selected from R^{6A}, R^{6B}, R^{6D}, R^{6E} and R^{6F} are taken together and are: —CH═CH—CH═CH₂— or —(CH₂)ₙ^{14a}— where n^{14a} is 3, 4 or 5 (e.g. 3 or 4).

In sub-formula (x), e.g. in sub-formula (x1), suitably, one, two or three of R^{6B}, R^{6D} and R^{6E} are other than a hydrogen atom (H).

In sub-formula (x), e.g. in sub-formula (x1), preferably, one or both of R^{6A} and R^{6F} are independently a hydrogen atom (H), a fluorine atom (F), or methyl. For example, one or both of R^{6A} and R^{6F} can be a hydrogen atom (H).

In sub-formula (x), e.g. in sub-formula (x1), suitably the ring or ring system is unsubstituted, monosubstituted, disubstituted or trisubstituted, preferably the ring or ring system is monosubstituted or disubstituted. In sub-formula (x), e.g. in sub-formula (x1), for monosubstitution of the ring or ring system, then the one substituent selected from R^{6A}, R^{6B}, R^{6D}, R^{6E} and R^{6F} is suitably present at the 3- or 4-position with respect to the —(CR⁴R⁵)— side-chain (i.e. D is CR^{6D} where R^{6D} is other than H), or is a 2-methyl 2-ethyl, 2-fluoro or 2-chloro substituent. In sub-formula (x), e.g. in sub-formula (x1), for disubstitution of the ring or ring system, then 3,4-disubstitution, 2,4-disubstitution, 2,3-disubstitution or 3,5-disubstitution is suitable.

In one preferable embodiment, Ar has the sub-formula (x1) and is: phenyl, monoalkyl-phenyl-, mono(fluoroalkyl)-phenyl-, monohalo-phenyl-, monoalkoxy-phenyl-, mono(fluoroalkoxy)-phenyl-, mono(N,N-dimethylamino)-phenyl-, mono(methyl-SO₂—NH-)-phenyl-, mono(methyl-SO₂-)-phenyl-, dialkyl-phenyl-, monoalkyl-monohalo-phenyl-, mono(fluoroalkyl)-monohalo-phenyl-, dihalo-phenyl-, dihalo-monoalkyl-phenyl-, dihalo-mono(hydroxymethyl)-phenyl- (e.g. 2,3-dichloro-6-(hydroxymethyl)-phenyl-), or dialkoxy-phenyl- such as 3,4-dimethoxy-phenyl-. The substituents can preferably be further defined, as defined in preferable embodiments herein.

In one preferable embodiment, Ar is of sub-formula (x1) and is: monoalkyl-phenyl-, mono(fluoroalkyl)-phenyl-, monohalo-phenyl-, monoalkoxy-phenyl-, mono(fluoroalkoxy)-phenyl-, dialkyl-phenyl-, monoalkyl-monohalo-phenyl-, dihalo-phenyl- or dihalo-monoalkyl-phenyl-.

More preferably, in this embodiment, Ar is:

monoC₁₋₄alkyl-phenyl- or monoC₁₋₃alkyl-phenyl- such as 4-C₁₋₄alkyl-phenyl- (e.g. 4-C₁₋₃alkyl-phenyl-) or 2-C₁₋₂alkyl-phenyl-;

monoC₁fluoroalkyl-phenyl- such as 4-C₁fluoroalkyl-phenyl-;

monoC₁₋₃alkoxy-phenyl- such as 4-C₁₋₃alkoxy-phenyl- or 3-C₁₋₃alkoxy-phenyl-;

mono(C₁fluoroalkoxy)-phenyl- such as 4-C₁fluoroalkoxy-phenyl-;

diC₁₋₃alkyl-phenyl- or diC₁₋₂alkyl-phenyl- or dimethyl-phenyl- such as 3,4-dimethyl-phenyl-, 2,4-dimethyl-phenyl-, 3,5-dimethyl-phenyl-, 2,3-dimethyl-phenyl- or 2,5-dimethyl-phenyl-; for example 3,4-dimethyl-phenyl-, 2,4-dimethyl-phenyl-, 2,3-dimethyl-phenyl- or 3,5-dimethyl-phenyl-;

monoC₁₋₃alkyl-monohalo-phenyl-, such as monoC₁₋₂alkyl-monohalo-phenyl- and/or monoC₁₋₃alkyl-monochloro-phenyl- or monoC₁₋₃alkyl-monofluoro-phenyl-, for example 4-methyl-3-chloro-phenyl-, 3-methyl-4-chloro-phenyl-, or 2-methyl-4-chloro-phenyl-;

dihalo-phenyl- such as 2-chloro-4-fluorophenyl- or 2,4-difluoro-phenyl- or 4-bromo-2-fluorophenyl- or preferably 4-chloro-2-fluorophenyl-; for example dichloro-phenyl- such as 3,4-dichloro-phenyl- or 2,4-dichloro-phenyl- or 2,6-dichloro-phenyl- or preferably 2,3-dichloro-phenyl-; or

dihalo-monoC₁₋₂alkyl-phenyl- e.g. 2,4-dichloro-6-methyl-phenyl-.

In an alternative embodiment, Ar has the sub-formula (z).

Preferably, in sub-formula (z), three or more (for example all) of J, L, M and Q are independently C—H, C—F, C—C₁₋₂alkyl (e.g. C-Me), C-[connection point to formula (I)], or nitrogen (N).

Preferably, in sub-formula (z), no more than two (for example no more than one) of J, L, M and Q are nitrogen (N).

Suitably, Q is C-[connection point to formula (I)].

Suitably, R⁹ is a hydrogen atom (H) or methyl.

Suitably, R^{6J}, R^{6L}, R^{6M} and/or R^{6Q} independently is or are: a hydrogen atom (H); fluoro; chloro; C₁₋₂alkyl (e.g. methyl); C₁fluoroalkyl (e.g. CF₃); C₁₋₂alkoxy(methoxy); C₁fluoroalkoxy (e.g. CF₂HO—); OH (including any tautomer thereof); or phenyl optionally substituted by one substituent being fluoro, methyl, C₁fluoroalkyl, methoxy or C₁fluoroalkoxy. More Suitably R^{6J}, R^{6L}, R^{6M} and/or R^{6Q} independently is or are H, OH (including any keto tautomer thereof), or more preferably C₁₋₂alkyl (e.g. methyl) or C₁fluoroalkyl.

When Ar has the sub-formula (z), then sub-formula (z) can suitably be one of the following:

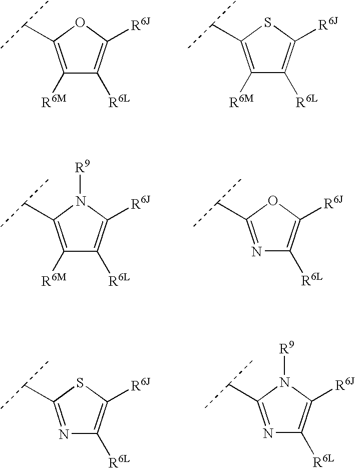

In another aspect of the invention Ar is phenyl and R^{6A}, R^{6B}, R^{6D}, R^{6E} and R^{6F} are independently H, C₁₋₂alkyl, C₁₋₂alkoxy or halogen.

Representative examples of Ar include 4-chloro-2-methylphenyl, 4-methoxyphenyl, 3,4-dimethylphenyl, 4-chlorophenyl, and phenyl.

Preferably, the compound of formula (I) or the salt thereof is a compound of formula (IA) or a salt thereof:

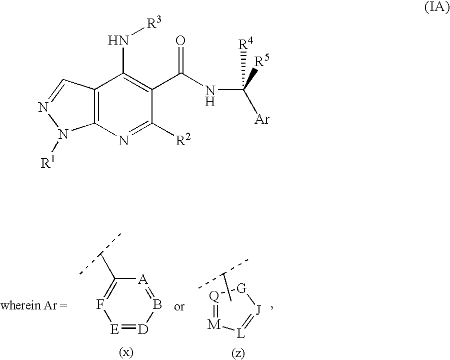

Formula (IA) means that more than 50% of the compound or salt present has the stereochemistry shown at the carbon atom bearing the R⁴ and R⁵ groups.

Preferably, the stereochemistry at the carbon atom bearing the R⁴ and R⁵ groups is such that there is an enantiomeric excess (e.e.) of 50% or more at the carbon atom bearing the R⁴ and R⁵ groups (ignoring the stereochemistry at any other carbon atoms). More preferably, the enantiomeric excess (e.e.) is 70% or more or 80% or more, still more preferably 90% or more, yet more preferably 95% or more, at the carbon atom bearing the R⁴ and R⁵ groups (ignoring the stereochemistry at any other carbon atoms).

“Enantiomeric excess” (e.e.) is defined as the percentage of the major isomer present minus the percentage of the minor isomer present. For example, if 95% of major isomer is present and 5% of the minor isomer is present, then the e.e. would be 90%.

In another aspect of the invention in formula (IA) R⁴ is not a hydrogen atom (H). In this aspect in formula (IA), R⁴ can for example be methyl, ethyl, C₁fluoroalkyl (such as CF₃), —CH₂OH, or —CH₂OMe.

In another aspect of the invention in formula (IA) R⁵ is a hydrogen atom (H) and R⁴ is not a hydrogen atom (H).

It is particularly preferred that the compound of formula (I) or the salt thereof is:

N-[(4-chloro-2-methylphenyl)methyl]-6-cyclopropyl-1-ethyl-4-(tetrahydro-2H-pyran-4-ylamino)-1H-pyrazolo[3,4-b]pyridine-5-carboxamide;

6-cyclopropyl-1-ethyl-N-(phenylmethyl)-4-(tetrahydro-2H-pyran-4-ylamino)-1H-pyrazolo[3,4-b]pyridine-5-carboxamide;

6-cyclopropyl-1-ethyl-N-{[4-(methyloxy)phenyl]methyl}-4-(tetrahydro-2H-pyran-4-ylamino)-1H-pyrazolo[3,4-b]pyridine-5-carboxamide;

6-cyclopropyl-N-[(3,4-dimethylphenyl)methyl]-1-ethyl-4-(tetrahydro-2H-pyran-4-ylamino)-1H-pyrazolo[3,4-b]pyridine-5-carboxamide;

N-[1-(4-chlorophenyl)ethyl]-6-cyclopropyl-1-ethyl-4-(tetrahydro-2H-pyran-4-ylamino)-1H-pyrazolo[3,4-b]pyridine-5-carboxamide;

N-[1-(4-chlorophenyl)propyl]-6-cyclopropyl-1-ethyl-4-(tetrahydro-2H-pyran-4-ylamino)-1H-pyrazolo[3,4-b]pyridine-5-carboxamide;

1-ethyl-N-(phenylmethyl)-6-propyl-4-(tetrahydro-2H-pyran-4-ylamino)-1H-pyrazolo[3,4-b]pyridine-5-carboxamide;

1-ethyl-N-{[4-(methyloxy)phenyl]methyl}-6-propyl-4-(tetrahydro-2H-pyran-4-ylamino)-1H-pyrazolo[3,4-b]pyridine-5-carboxamide;

N-[(4-chloro-2-methylphenyl)methyl]-1-ethyl-6-propyl-4-(tetrahydro-2H-pyran-4-ylamino)-1H-pyrazolo[3,4-b]pyridine-5-carboxamide;

N-[(3,4-dimethylphenyl)methyl]-1-ethyl-6-propyl-4-(tetrahydro-2H-pyran-4-ylamino)-1H-pyrazolo[3,4-b]pyridine-5-carboxamide;

N-(2,3-dihydro-1H-inden-2-yl)-1-ethyl-6-propyl-4-(tetrahydro-2H-pyran-4-ylamino)-1H-pyrazolo[3,4-b]pyridine-5-carboxamide;

1,6-diethyl-N-(phenylmethyl)-4-(tetrahydro-2H-pyran-4-ylamino)-1H-pyrazolo[3,4-b]pyridine-5-carboxamide;

1,6-diethyl-N-{[4-(methyloxy)phenyl]methyl}-4-(tetrahydro-2H-pyran-4-ylamino)-1H-pyrazolo[3,4-b]pyridine-5-carboxamide;

N-[(3,4-dimethylphenyl)methyl]-1,6-diethyl-4-(tetrahydro-2H-pyran-4-ylamino)-1H-pyrazolo[3,4-b]pyridine-5-carboxamide;

N-(2,3-dihydro-1H-inden-2-yl)-1,6-diethyl-4-(tetrahydro-2H-pyran-4-ylamino)-1H-pyrazolo[3,4-b]pyridine-5-carboxamide;

N-[1-(4-chlorophenyl)propyl]-1,6-diethyl-4-(tetrahydro-2H-pyran-4-ylamino)-1H-pyrazolo[3,4-b]pyridine-5-carboxamide;

6-cyclobutyl-1-ethyl-N-(phenylmethyl)-4-(tetrahydro-2H-pyran-4-ylamino)-1H-pyrazolo[3,4-b]pyridine-5-carboxamide;

6-cyclobutyl-1-ethyl-N-{[4-(methyloxy)phenyl]methyl}-4-(tetrahydro-2H-pyran-4-ylamino)-1H-pyrazolo[3,4-b]pyridine-5-carboxamide;

6-(cyclopropylmethyl)-N-[(3,4-dimethylphenyl)methyl]-1-ethyl-4-(tetrahydro-2H-pyran-4-ylamino)-1H-pyrazolo[3,4-b]pyridine-5-carboxamide;

6-cyclobutyl-N-(2,3-dihydro-1H-inden-2-yl)-1-ethyl-4-(tetrahydro-2H-pyran-4-ylamino)-1H-pyrazolo[3,4-b]pyridine-5-carboxamide;

N-[(4-chloro-2-methylphenyl)methyl]-6-cyclobutyl-1-ethyl-4-(tetrahydro-2H-pyran-4-ylamino)-1H-pyrazolo[3,4-b]pyridine-5-carboxamide;

N-[1-(4-chlorophenyl)ethyl]-6-cyclobutyl-1-ethyl-4-(tetrahydro-2H-pyran-4-ylamino)-1H-pyrazolo[3,4-b]pyridine-5-carboxamide;

N-[1-(4-chlorophenyl)propyl]-6-cyclobutyl-1 ethyl-4-(tetrahydro-2H-pyran-4-ylamino)-1H-pyrazolo[3,4-b]pyridine-5-carboxamide;

6-(cyclopropylmethyl)-1-ethyl-N-(phenylmethyl)-4-(tetrahydro-2H-pyran-4-ylamino)-1H-pyrazolo[3,4-b]pyridine-5-carboxamide;

6-(cyclopropylmethyl)-N-[(3,4-dimethylphenyl)methyl]-1-ethyl-4-(tetrahydro-2H-pyran 4-ylamino)-1H-pyrazolo[3,4-b]pyridine-5-carboxamide;

6-(cyclopropylmethyl)-N-(2,3-dihydro-1H-inden-2-yl)-1-ethyl-4-(tetrahydro-2H-pyran-4-ylamino)-1H-pyrazolo[3,4-b]pyridine-5-carboxamide;

N-[1-(4-chlorophenyl)ethyl]-6-(cyclopropylmethyl)-1-ethyl-4-(tetrahydro-2H-pyran-4-ylamino)-1H-pyrazolo[3,4-b]pyridine-5-carboxamide;

6-cyclopentyl-1-ethyl-N-(phenylmethyl)-4-(tetrahydro-2H-pyran-4-ylamino)-1H-pyrazolo[3,4-b]pyridine-5-carboxamide; or

6-cyclopentyl-N-(2,3-dihydro-1H-inden-2-yl)-1-ethyl-4-(tetrahydro-2H-pyran-4-ylamino)-1H-pyrazolo[3,4-b]pyridine-5-carboxamide.

or a salt thereof, e.g. a pharmaceutically acceptable salt thereof.

The structures of these specific compounds are given in Examples 1 to 29 hereinafter.

Alternatively, it is particularly preferred that the compound of formula (I) or the salt thereof is one of Examples 1 to 29, as a compound or a salt thereof, e.g. a pharmaceutically acceptable salt thereof. The structures of these specific compounds are given in Examples 1 to 29 hereinafter, and their names are given in the Examples section.

In a further aspect the invention provides a compound of formula (I) or a salt thereof selected from the group consisting of:

N-[(4-chloro-2-methylphenyl)methyl]-6-cyclopropyl-1-ethyl-4-(tetrahydro-2H-pyran-4-ylamino)-1H-pyrazolo[3,4-b]pyridine-5-carboxamide;

6-cyclopropyl-1-ethyl-N-(phenylmethyl)-4-(tetrahydro-2H-pyran-4-ylamino)-1H-pyrazolo[3,4-b]pyridine-5-carboxamide;

6-cyclopropyl-1-ethyl-N-{[4-(methyloxy)phenyl]methyl}-4-(tetrahydro-2H-pyran-4-ylamino)-1H-pyrazolo[3,4-b]pyridine-5-carboxamide;

6-cyclopropyl-N-[(3,4-dimethylphenyl)methyl]-1-ethyl-4-(tetrahydro-2H-pyran-4-ylamino)-1H-pyrazolo[3,4-b]pyridine-5-carboxamide;

N-[1-(4-chlorophenyl)ethyl]-6-cyclopropyl-1-ethyl-4-(tetrahydro-2H-pyran-4-ylamino)-1H-pyrazolo[3,4-b]pyridine-5-carboxamide; or

N-[1-(4-chlorophenyl)propyl]-6-cyclopropyl-1-ethyl-4-(tetrahydro-2H-pyran-4-ylamino)-1H-pyrazolo[3,4-b]pyridine-5-carboxamide;

or a salt thereof, e.g. a pharmaceutically acceptable salt thereof.

Because of their potential use in medicine, the salts of the compounds of formula (I) are preferably pharmaceutically acceptable. Suitable pharmaceutically acceptable salts can include acid or base addition salts.

A pharmaceutically acceptable acid addition salt can be formed by reaction of a compound of formula (I) with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

A pharmaceutically acceptable base addition salt can be formed by reaction of a compound of formula (I) with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the the compound of formula (I).

Other non-pharmaceutically acceptable salts, eg. oxalates, may be used, for example in the isolation of compounds of the invention, and are included within the scope of this invention.

The invention includes within its scope all possible stoichiometric and non-stoichiometric forms of the salts of the compounds of formula (I).

Also included within the scope of the invention are all solvates, hydrates and complexes of compounds and salts of the invention.

Certain groups, substituents, compounds or salts included in the present invention may be present as isomers. The present invention includes within its scope all such isomers, including racemates, enantiomers and mixtures thereof.

In the compounds or salts, pharmaceutical compositions, uses, methods of treatment/prophylaxis, methods of preparing, etc. according to the present invention, where a defined isomeric configuration e.g. stereochemical configuration is described or claimed, the invention includes a mixture comprising (a) a major component of the compound or salt which is in the described or claimed configuration, together with (b) one or more minor components of the compound or salt which is/are not in the described or claimed configuration. Preferably, in such a mixture, the major component of the compound or salt which is in the described or claimed configuration represents 70% or more, or 75% or more, more preferably 85% or more, still more preferably 90% or more, yet more preferably 95% or more, yet more preferably 98% or more, of the total amount of compound or salt present in the mixture on a molarity basis.

The percentage of one isomeric/stereochemical component in a mixture of different isomeric/stereochemical components, and if appropriate enantiomeric and/or diastereomeric excesses, can be measured using techniques known in the art. Such methods include the following:

(1) Measurement using NMR (e.g. ¹H NMR) spectroscopy in the presence of chiral agent. One can measure a nuclear magnetic resonance (NMR) spectrum (preferably a ¹H NMR spectrum, and/or a solution-phase NMR spectrum e.g. in CDCl₃ or D6-DMSO solvent) of the compound/salt mixture in the presence of a suitable chiral agent which “splits” the NMR peaks of a given atom in different isomers into different peak positions. The chiral agent can be: i) an optically pure reagent which reacts with the compound/salt e.g. to form a mixture of diastereomers, ii) a chiral solvent, iii) a chiral molecule which forms a transient species (e.g. diastereomeric species) with the compound/salt, or iv) a chiral shift reagent. See e.g. J. March, “Advanced Organic Chemistry”, 4th edn., 1992, pages 125-126 and refs. 138-146 cited therein. A chiral shift reagent can be a chiral lanthanide shift reagent such as tris[3-trifluoroacetyl-d-camphorato]europium-(III) or others as described in Morrill, “Lanthanide Shift Reagents in Stereochemical Analysis”, VCH, New York, 1986. Whatever the chiral agent is that is used, usually, the relative integrals (intensities) for the NMR peaks of a given atom or group in different isomers can provide a measurement of the relative amounts of each isomer present.

(2) Measurement using chiral chromatography, especially on an analytical scale. A suitable chiral column which separates the different isomeric components can be used to effect separation, e.g. using gas or liquid chromatography such as HPLC, and/or e.g. on an analytical scale. The peaks for each isomer can be integrated (area under each peak); and a comparison or ratio of the integrals for the different isomers present can give a measurement of the percentage of each isomeric component present. See for example: “Chiral Chromatography”, Separation Science Series Author: T. E. Beesley and R. P. W. Scott, John Wiley & Sons, Ltd., Chichester, UK, 1998, electronic Book ISBN: 0585352690, Book ISBN: 0471974277.

(3) Separation of pre-existing diastereomeric mixtures which are compounds/salts of the invention can be achieved (usually directly, without derivatisation) using separation techniques such as gas or liquid chromatography. Diastereomeric ratios and/or excesses can thereby be derived e.g. from the relative peak areas or relative separated masses.

(4) Conversion with a chiral/optically-active agent and subsequent separation of the resulting isomers, e.g. diastereomers. Conversion can be via derivatisation of a derivatisable group (e.g. —OH, —NHR) on the compound/salt with an optically-active derivatising group (e.g. optically active acid chloride or acid anhydride); or can be via formation of an acid or base addition salt of the compound by treatment of the compound with an optically-active acid or base, such as + or − di-para-toluoyl tartaric acid. After derivatisation, separation of the resulting isomers e.g. diastereomers, can be using gas or liquid chromatography (usually non-chiral); or (especially with isomeric salts) can be by selective crystallisation of a single isomeric e.g. diastereoisomeric salt. Determination of isomeric ratios and/or excesses can be using chromatography peak areas or measurement of mass of each separated isomer.

See e.g. J. March, “Advanced Organic Chemistry”, 4th edn., 1992, pages 120-121 and 126, and refs. 105-115 and 147-149 cited therein.

(5) Measurement of optical activity [alpha] of mixture and comparison with optical activity of pure isomer [alpha]ₘₐₓ if available (e.g. see J. March, “Advanced Organic Chemistry”, 4th edn., 1992, page 125 and refs. 138-139 cited therein). This assumes a substantially linear relationship between [alpha] and concentration.

Certain of the groups, e.g. heteroaromatic ring systems, included in compounds of formula (I) or their salts may exist in one or more tautomeric forms. The present invention includes within its scope all such tautomeric forms, including mixtures.

Especially when intended for oral medicinal use, the compound of formula (I) can optionally have a molecular weight of 1000 or less, for example 800 or less, in particular 650 or less or 600 or less. Molecular weight here refers to that of the unsolvated “free base” compound, that is excluding any molecular weight contributed by any addition salts, solvent (e.g. water) molecules, etc.

The following processes can be used to make the compounds of the invention: 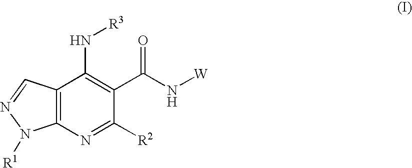
Process A

To form a compound of formula (I) wherein W═—CR⁴R⁵Ar, a carboxylic acid of formula (II) can be converted into an activated compound of formula (III) wherein X¹=a leaving group substitutable by an amine (as defined below) and subsequently the activated compound can be reacted with an amine of formula ArCR⁴R⁵NH₂:

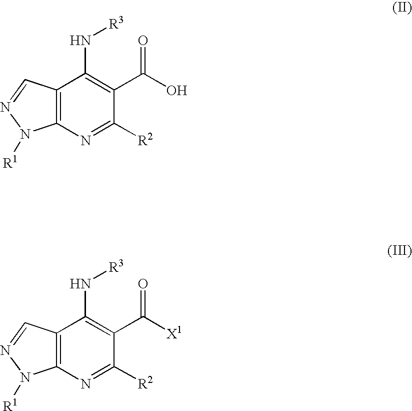

For example, the activated compound (the compound of formula (III)) can be the acid chloride. This can be formed from the carboxylic acid (II) e.g. by reaction with thionyl chloride, either in an organic solvent such as chloroform or without solvent. Alternatively, the activated compound (the compound of formula (III)) can be an activated ester wherein the leaving group X¹ is

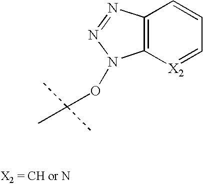

The latter activated compound of formula (III) can be formed from the carboxylic acid (II) either:

(a) by reaction of the carboxylic acid with a carbodiimide such as EDC, which is 1-ethyl-3-(3′-dimethylaminopropyl)carbodiimide and is also 1-(3-dimethylaminopropyl)-3-ethylcarbodiimide, or a salt thereof e.g. hydrochloride salt, preferably followed by reaction of the resulting product with 1-hydroxybenzotriazole (HOBT); reaction (a) usually being carried out in the presence of a solvent (preferably anhydrous) such as dimethyl formamide (DMF) or acetonitrile and/or preferably under anhydrous conditions and/or usually at room temperature (e.g. about 20 to about 25° C.); or:

(b) by reaction with 2-(1H-benzotriazole-1-yl)-1,1,3,3-tetramethyluronium tetrafluoroborate (TBTU) or O-(7-Azabenzotriazol-1-yl)-N,N,N′,N′-tetramethyluronium hexafluorophosphate (HATU), in the presence of a base such as diisopropylethylamine (ⁱPr₂NEt=DIPEA), and usually in the presence of a solvent such as dimethyl formamide (DMF) or acetonitrile and/or preferably under anhydrous conditions and/or usually at room temperature (e.g. about 20 to about 25° C.).

Compounds of formula (II) can be prepared by hydrolysis of an ester of formula (IV). This procedure preferably involves reaction of (IV) with either:
(a) a base such as sodium hydroxide or potassium hydroxide in a solvent e.g. an aqueous solvent such as aqueous ethanol or aqueous dioxane or
(b) an acid such as hydrochloric acid in a solvent e.g. an aqueous solvent such as aqueous dioxane:

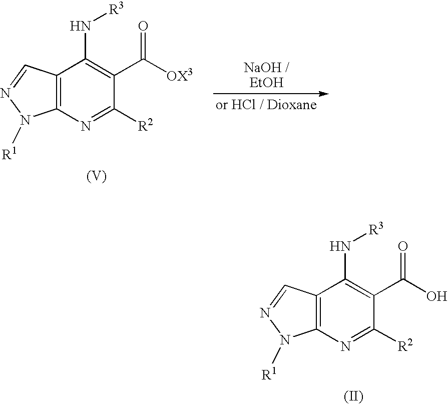

Compounds of formula (IV) can be prepared according to a method, for example as described by Yu et. al. in J. Med Chem., 2001, 44, 1025-1027, by reaction of a compound of formula (V) with an amine of formula R³NH₂. The reaction is preferably carried out in the presence of a base such as triethylamine or N,N-diisopropylethylamine, and/or in an organic solvent such as ethanol, dioxane, 1-methyl-2-pyrrolidinone (NMP) or acetonitrile. The reaction may require heating e.g. to ca. 60-180° C., for example ca. 140-160° C.:

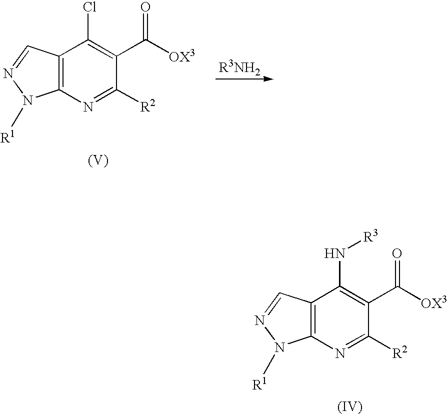

Compounds of formula (V) are also described in the above reference and can be prepared by reaction of a compound of formula (VI) with, for example, diethyl[chloro(cyclopropyl)methylene]malonate (VII) (where X³=Et) with heating, followed by reaction with phosphorous oxychloride, again with heating:

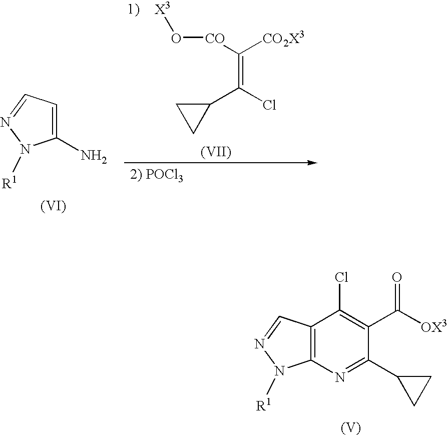

Compounds of formula (VII) are also described in the above reference and can be prepared by reaction of a malonate derivative, for example diethyl(cyclopropylcarbonyl)malonate (VIII), with phosphorus oxychloride in the presence of a base such as tributylamine. The reaction may require heating, e.g. to ca 80-130° C., for example ca. 100-120° C.:

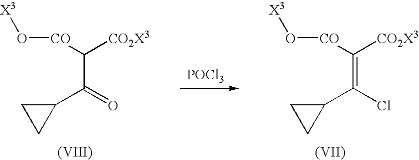

Compounds of formula (VII) are also described in the above reference and can be prepared by reaction of diethyl malonate (IX) with magnesium powder followed by an acid chloride, for example cyclopropylcarbonyl chloride (X).

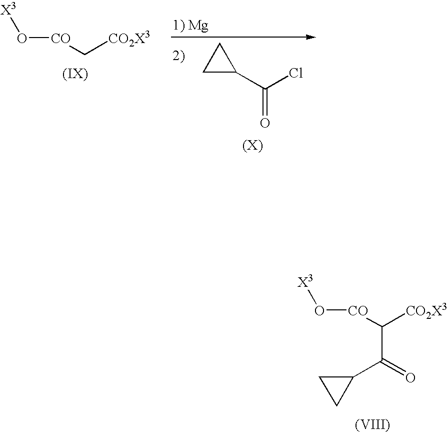

Where the desired amino pyrazole of formula (VI) is not commercially available, preparation can be achieved using methods described by Dorgan et. al. in J. Chem. Soc., Perkin Trans. 1, (4), 938-42; 1980, by reaction of cyanoethylhydrazine with a suitable aldehyde of formula R⁴⁰CHO in a solvent such as ethanol, with heating, followed by reduction with, for example sodium in a solvent such as t-butanol. R⁴⁰ should be chosen so as to contain one less carbon atom than R¹, for example R⁴⁰=methyl will afford R¹=ethyl.

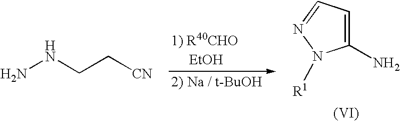

In an alternative embodiment of Process A, the 4-chloro substituent in the compound of formula (V) can be replaced by another halogen atom, such as a bromine atom, or by another suitable leaving group which is displaceable by an amine of formula R³NH₂. The leaving group can, for example, be an alkoxy group —OR³⁵ such as —OC₁₋₄alkyl (in particular —OEt) or a group —O—S(O)₂—R³⁷, wherein R³⁷ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction may be carried out with or without solvent and may require heating.

Process B

Compounds of formula (I) can be prepared by reaction of a compound of formula (XI) with an amine of formula R³NH₂. The reaction is preferably carried out in the presence of a base, such as triethylamine or N,N-diisopropylethylamine, and/or in an organic solvent such as ethanol, THF, dioxane or acetonitrile. The reaction may require heating, e.g. to ca. 60-100° C. or ca. 80-90° C., for example for 8-48 or 12-24 hours:

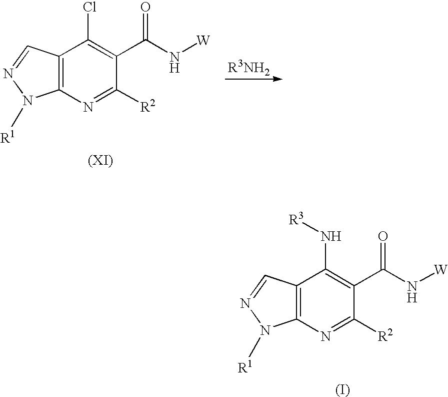

Compounds of formula (XI) can be prepared in a two step procedure as described by Bare et. al. in J. Med. Chem. 1989, 32, 2561-2573. This process involves, first, reaction of a compound of formula (XII) with thionyl chloride (or another agent suitable for forming an acid chloride from a carboxylic acid), either in an organic solvent such as chloroform or THF, or as a neat solution. This reaction may require heating and the thus-formed intermediate may or may not be isolated. Step two involves reaction with an amine of formula WNH₂, in an organic solvent such as THF or chloroform and may also involve the use of a base such as triethylamine or diisopropylethylamine:

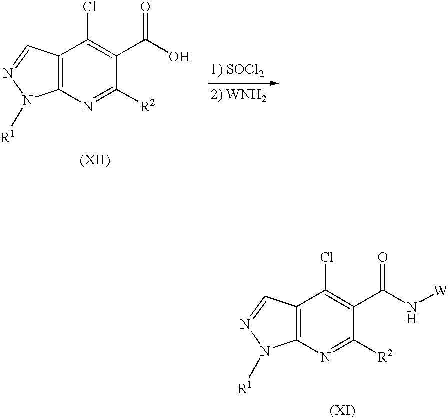

Compounds of formula (XII) can be prepared by hydrolysis of an ester of formula (V) according to the method described by Yu et. al. in J. Med Chem., 2001, 44, 1025-1027. This procedure preferably involves reaction with a base such as sodium hydroxide or potassium hydroxide in a solvent e.g. an aqueous solvent such as aqueous ethanol or aqueous dioxane:

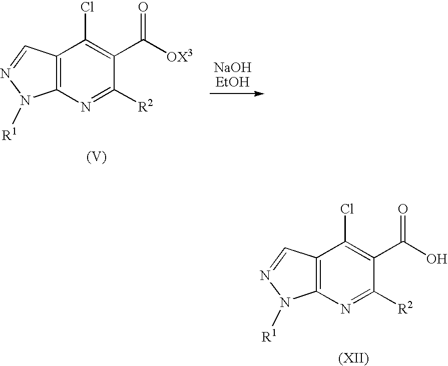

In an alternative embodiment of Process B, the 4-chloro substituent in the compound of formula (IV) can be replaced by another halogen atom, such as a bromine atom.

Process C

Compounds of formula (IV) can be prepared by reaction of a compound of formula (XIII) with an alkylating agent of formula R¹—X⁴, where X⁴ is a leaving group displaceable by the 1-position pyrazolopyridine nitrogen atom of the compound of formula (XIII):

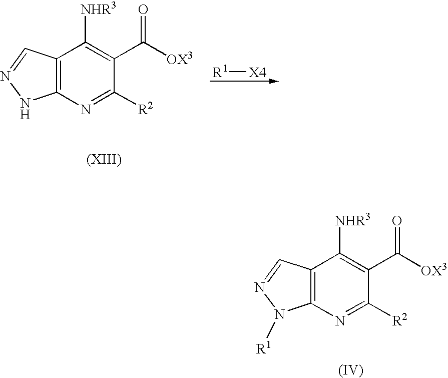

A suitable alkylating agent of formula R¹—X⁴ can be used. For example, X⁴ can be a halogen atom such as a chlorine atom or more preferably a bromine or iodine atom, or X³ can be —O—S(O)₂—R³⁶ wherein R³⁶ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction is preferably carried out in the presence of a base; the base can for example comprise or be potassium carbonate, sodium carbonate, sodium hydride, potassium hydride, or a basic resin or polymer such as polymer-bound 2-tert-butylimino-2-diethylamino-1,3-dimethyl-perhydro-1,3,2-diazaphosphorine. The reaction is preferably carried out in the presence of a solvent, e.g. an organic solvent such as DMF; the solvent is preferably anhydrous. Compounds of formula (XIII) can be prepared, using a method analogous to that used for the preparation of compounds (IV), by reaction of a compound of formula (V) (R¹═H) with an amine of formula R³NH₂. The reaction is preferably carried out in the presence of a base such as triethylamine or N,N-diisopropylethylamine, and/or in an organic solvent such as ethanol, dioxane or acetonitrile. The reaction may require heating e.g. to ca. 60-100° C., for example ca. 80-90° C.: 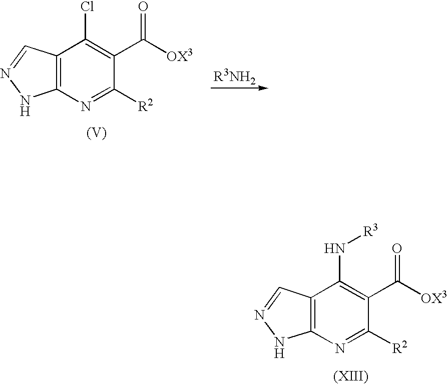
Process D: Conversion of One Compound of Formula (I)-(IV) or Salt thereof into Another Compound of Formula (I)-(IV) or Salt thereof

One compound of formula (I)-(IV) or salt thereof can be converted into another compound of formula (I)-(IV) or salt thereof. This conversion preferably comprises or is one or more of the following processes D1 to D7:
D1. Conversion of a ketone into the corresponding oxime.
D2. An oxidation process. For example, the oxidation process can comprise or be oxidation of an alcohol to a ketone (e.g. using Jones reagent) or oxidation of an alcohol or a ketone to a carboxylic acid.
D3. A reduction process, for example reduction of a ketone or a carboxylic acid to an alcohol.
D4. Alkylation, for example alkylation of an amine or of a hydroxy group.
D5. Hydrolysis, e.g. hydrolysis of an ester to the corresponding carboxylic acid or salt thereof.
D6. Deprotection, e.g. deprotection (e.g. deacylation or t-butyloxycarbonyl (BOC) removal) of an amine group.
D7. Formation of an ester or amide, for example from the corresponding carboxylic acid.

The present invention also provides a compound of formula (I) or a pharmaceutically acceptable salt thereof for use as an active therapeutic substance in a mammal such as a human. The compound or salt can be for use in the treatment and/or prophylaxis of any of the diseases/conditions described herein (e.g. for use in the treatment and/or prophylaxis of an inflammatory and/or allergic disease in a mammal) and/or for use as a phosphodiesterase inhibitor e.g. for use as a phosphodiesterase 4 (PDE4) inhibitor. “Therapy” may include treatment and/or prophylaxis.

Also provided is the use of a compound of formula (I) or a pharmaceutically acceptable salt thereof in the manufacture of a medicament (e.g. pharmaceutical composition) for the treatment and/or prophylaxis of any of the diseases/conditions described herein in a mammal such as a human, e.g. for the treatment and/or prophylaxis of an inflammatory and/or allergic disease in a mammal such as a human.

Also provided is a method of treatment and/or prophylaxis of any of the diseases/conditions described herein in a mammal (e.g. human) in need thereof, e.g. a method of treatment and/or prophylaxis of an inflammatory and/or allergic disease in a mammal (e.g. human) in need thereof, which method comprises administering to the mammal (e.g. human) a therapeutically effective amount of a compound of formula (I) as herein defined or a pharmaceutically acceptable salt thereof.

Phosphodiesterase 4 inhibitors are thought to be useful in the treatment and/or prophylaxis of a variety of diseases/conditions, especially inflammatory and/or allergic diseases, in mammals such as humans, for example: asthma, chronic obstructive pulmonary disease (COPD) (e.g. chronic bronchitis and/or emphysema), atopic dermatitis, urticaria, allergic rhinitis, allergic conjunctivitis, vernal conjunctivitis, eosinophilic granuloma, psoriasis, rheumatoid arthritis, septic shock, ulcerative colitis, Crohn's disease, reperfusion injury of the myocardium and brain, chronic glomerulonephritis, endotoxic shock, adult respiratory distress syndrome, multiple sclerosis, cognitive impairment (e.g. in a neurological disorder such as Alzheimer's disease), depression, or pain. Ulcerative colitis and/or Crohn's disease are collectively often referred to as inflammatory bowel disease.

In the treatment and/or prophylaxis, the inflammatory and/or allergic disease is preferably chronic obstructive pulmonary disease (COPD), asthma, rheumatoid arthritis or allergic rhinitis in a mammal (e.g. human). More preferably, the treatment and/or prophylaxis is of COPD or asthma in a mammal (e.g. human).

PDE4 inhibitors are thought to be effective in the treatment of asthma (e.g. see M. A. Giembycz, Drugs, February 2000, 59(2), 193-212; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; and references cited in the aforementioned publications).

PDE4 inhibitors are thought to be effective in the treatment of COPD. For example, see S. L. Wolda, Emerging Drugs, 2000, 5(3), 309-319; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; and references cited in the aforementioned publications; and G. Krishna et al., Expert Opinion on Investigational Drugs, 2004, 13(3), 255-267 (see especially pp. 259-261 and refs. 102-111 and 201 therein). COPD is often characterised by the presence of airflow obstruction due to chronic bronchitis and/or emphysema (e.g., see S. L. Wolda, Emerging Drugs, 2000, 5(3), 309-319).

PDE4 inhibitors are thought to be effective in the treatment of allergic rhinitis (e.g. see B. M. Schmidt et al., J. Allergy & Clinical Immunology, 108(4), 2001, 530-536).

PDE4 inhibitors are thought to be effective in the treatment of rheumatoid arthritis and multiple sclerosis (e.g. see H. J. Dyke et al., Expert Opinion on Investigational Drugs, January 2002, 11(1), 1-13; C. Burnouf et al., Current Pharmaceutical Design, 2002, 8(14), 1255-1296; and A. M. Doherty, Current Opinion Chem. Biol., 1999, 3(4), 466-473; and references cited in these publications). See e.g. A. M. Doherty, Current Opinion Chem. Biol., 1999, 3(4), 466-473 and references cited therein for atopic dermatitis use.

PDE4 inhibitors have been suggested as having analgesic properties and thus being effective in the treatment of pain (A. Kumar et al., Indian J. Exp. Biol., 2000, 38(1), 26-30).

In the invention, the treatment and/or prophylaxis can be of cognitive impairment e.g. cognitive impairment in a neurological disorder such as Alzheimer's disease. For example, the treatment and/or prophylaxis can comprise cognitive enhancement e.g. in a neurological disorder. See for example: H. T. Zhang et al. in: Psychopharmacology, June 2000, 150(3), 311-316 and Neuropsychopharmacology, 2000, 23(2), 198-204; and T. Egawa et al., Japanese J. Pharmacol., 1997, 75(3), 275-81.

PDE4 inhibitors such as rolipram have been suggested as having antidepressant properties (e.g. J. Zhu et al., CNS Drug Reviews, 2001, 7(4), 387-398; O'Donnell, Expert Opinion on Investigational Drugs, 2000, 9(3), 621-625; and H. T. Zhang et al., Neuropsychopharmacology, October 2002, 27(4), 587-595).

For use in medicine, the compounds of the present invention are usually administered as a pharmaceutical composition.

The present invention therefore provides in a further aspect a pharmaceutical composition comprising a compound of formula (I) or a pharmaceutically acceptable salt thereof and one or more pharmaceutically acceptable carriers and/or excipients.

The pharmaceutical composition can be for use in the treatment and/or prophylaxis of any of the conditions described herein.

The invention also provides a method of preparing a pharmaceutical composition comprising a compound of formula (I), as herein defined, or a pharmaceutically acceptable salt thereof, and one or more pharmaceutically acceptable carriers and/or excipients,

the method comprising mixing the compound or salt with the one or more pharmaceutically acceptable carriers and/or excipients.

The invention also provides a pharmaceutical composition prepared by said method.

The compounds of formula (I) and/or the pharmaceutical composition may be administered, for example, by oral, parenteral (e.g. intravenous, subcutaneous, or intramuscular), inhaled or nasal administration. Accordingly, the pharmaceutical composition is preferably suitable for oral, parenteral (e.g. intravenous, subcutaneous, or intramuscular), inhaled or nasal administration. More preferably, the pharmaceutical composition is suitable for inhaled or oral administration, e.g. to a mammal such as a human. Inhaled administration involves topical administration to the lung e.g. by aerosol or dry powder composition. Oral administration to a human is most preferred.

A pharmaceutical composition suitable for oral administration can be liquid or solid; for example it can be a syrup, suspension or emulsion, a tablet, a capsule or a lozenge.

A liquid formulation will generally consist of a suspension or solution of the compound or pharmaceutically acceptable salt in a suitable pharmaceutically acceptable liquid carrier(s), for example an aqueous solvent such as water, ethanol or glycerine, or a non-aqueous solvent, such as polyethylene glycol or an oil. The formulation may also contain a suspending agent, preservative, flavouring and/or colouring agent.

A pharmaceutical composition suitable for oral administration being a tablet can comprise one or more pharmaceutically acceptable carriers and/or excipients suitable for preparing tablet formulations. The carrier can for example be or include lactose, cellulose (for example microcrystalline cellulose), or mannitol. The tablet can also or instead contain one or more pharmaceutically acceptable excipients, for example a binding agent such as hydroxypropylmethylcellulose or povidone (polyvinylpyrollidone), a lubricant e.g. an alkaline earth metal stearate such as magnesium stearate, and/or a tablet disintegrant such as sodium starch glycollate, croscarmellose sodium, or crospovidone (cross-linked polyvinylpyrollidone). The pharmaceutical composition being a tablet can be prepared by a method comprising the steps of: (i) mixing the compound of formula (I), as herein defined, or a pharmaceutically acceptable salt thereof, with the one or more pharmaceutically acceptable carriers and/or excipients, (ii) compressing the resulting mixture (which is usually in powder form) into tablets, and (iii) optionally coating the tablet with a tablet film-coating material.

A pharmaceutical composition suitable for oral administration being a capsule can be prepared using encapsulation procedures. For example, pellets or powder containing the active ingredient can be prepared using a suitable pharmaceutically acceptable carrier and then filled into a hard gelatin capsule. Alternatively, a dispersion or suspension can be prepared using any suitable pharmaceutically acceptable carrier, for example an aqueous gum or an oil and the dispersion or suspension then filled into a soft gelatin capsule.

Preferably the composition is in unit dose form such as a tablet or capsule for oral administration, e.g. for oral administration to a human.

A parenteral composition can comprise a solution or suspension of the compound or pharmaceutically acceptable salt in a sterile aqueous carrier or parenterally acceptable oil. Alternatively, the solution can be lyophilised; the lyophilised parenteral pharmaceutical composition can be reconstituted with a suitable solvent just prior to administration.

Compositions for nasal or inhaled administration may conveniently be formulated as aerosols, drops, gels or dry powders.

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide, or an organic propellant such as a chlorofluorocarbon (CFC) or hydrofluorocarbon (HFC). Suitable CFC propellants include dichlorodifluoromethane, trichlorofluoromethane and dichlorotetrafluoroethane. Suitable HFC propellants include 1,1,1,2,3,3,3-heptafluoropropane and 1,1,1,2-tetrafluoroethane. The aerosol dosage forms can also take the form of a pump-atomiser.

For pharmaceutical compositions suitable and/or adapted for inhaled administration, it is preferred that the compound or salt of formula (I) is in a particle-size-reduced form, and more preferably the size-reduced form is obtained or obtainable by micronisation. Micronisation usually involves subjecting the compound/salt to collisional and/or abrasional forces in a fast-flowing circular or spiral/vortex-shaped airstream often including a cyclone component. The preferable particle size of the size-reduced (e.g. micronised) compound or salt is defined by a D50 value of about 0.5 to about 10 microns, e.g. about 1 to about 7 microns (e.g. as measured using laser diffraction). For example, it is preferable for the compound or salt of formula (I) to have a particle size defined by: a D10 of about 0.3 to about 3 microns (e.g. about 0.5 to about 2 microns, or about 1 micron), and/or a D50 of about 0.5 to about 10 microns or about 1 to about 7 microns (e.g. about 2 to about 5 microns or about 2 to about 4 microns), and/or a D90 of about 1 to about 30 microns or about 2 to about 20 microns or about 3 to about 15 microns (e.g. about 5 to about 15 microns or about 5 to about 10 microns); for example as measured using laser diffraction.

In particle size measurements, D90, D50 and D10 respectively mean that 90%, 50% and 10% of the material is less than the micron size specified. D50 is the median particle size. DV90, DV50 and DV10 respectively mean that 90%, 50% and 10% by volume of the material is less than the micron size specified. DM90, DM50 and DM10 respectively mean that 90%, 50% and 10% by weight of the material is less than the micron size specified.

Laser diffraction measurement of particle size can use a dry method (wherein a suspension of the compound/salt in an airflow crosses the laser beam) or a wet method [wherein a suspension of the compound/salt in a liquid dispersing medium, such as isooctane or (e.g. if compound is soluble in isooctane) 0.1% Tween 80 in water, crosses the laser beam]. With laser diffraction, particle size is preferably calculated using the Fraunhofer calculation; and/or preferably a Malvern Mastersizer or Sympatec apparatus is used for measurement. For example, particle size measurement and/or analysis by laser diffraction can use any or all of (preferably all of) the following: a Malvern Mastersizer longbed version, a dispersing medium of 0.1% Tween 80 in water, a stir rate of ca. 1500 rpm, ca. 3 mins sonification prior to final dispersion and analysis, a 300 RF (Reverse Fourier) lens, and/or the Fraunhofer calculation with Malvern software.

For pharmaceutical compositions suitable and/or adapted for inhaled administration, it is preferred that the pharmaceutical composition is a dry powder inhalable composition. Such a composition can comprise a powder base such as lactose or starch, the compound of formula (I) or salt thereof (preferably in particle-size-reduced form, e.g. in micronised form), and optionally a performance modifier such as L-leucine, mannitol, trehalose and/or magnesium stearate. Preferably, the dry powder inhalable composition comprises a dry powder blend of lactose and the compound of formula (I) or salt thereof. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

In the dry powder inhalable composition, preferably, the compound of formula (I) or salt thereof is present in about 0.1% to about 70% (e.g. about 1% to about 50%, e.g. about 5% to about 40%, e.g. about 20 to about 30%) by weight of the composition.

An illustrative non-limiting example of a dry powder inhalable composition follows:

Dry Powder Formulation Example—Dry Powder Lactose Blend Preparation

Using a size-reduced e.g. micronised form of the compound of formula (I) or salt thereof, the dry powder blend is prepared by mixing the required amount of the compound/salt (e.g. 10 mg, 1% w/w) with inhalation-grade lactose containing 10% fines (e.g. 990 mg, 99% w/w) in a Teflon™ (polytetrafluoroethene) pot in a Mikro-dismembrator ball-mill (but without a ball bearing) at 3/4 speed (ca. 2000-2500 rpm) for about 4 hours at each blend concentration. The Mikro-dismembrator (available from B. Braun Biotech International, Schwarzenberger Weg 73-79, D-34212 Melsungen, Germany; www.bbraunbiotech.com) comprises a base with an upwardly-projecting and sidewardly-vibratable arm to which is attached the Teflon™ pot. The vibration of the arm achieves blending.

Other blends: 10% w/w compound/salt (50 mg)+90% w/w lactose (450 mg, inhalation-grade lactose containing 10% fines).

Serial dilution ofthe 1% w/w blend can achieve e.g. 0.1% and 0.3% w/w blends.

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose, e.g. of the dry powder composition, can be administered by inhalation via a device such as the DISKUS™ device, marketed by GlaxoSmithKline. The DISKUS™ inhalation device is usually substantially as described in GB 2,242,134 A. In such device at least one container for the pharmaceutical composition in powder form (the at least one container preferably being a plurality of sealed dose containers mounted longitudinally in a strip or ribbon) is defined between two members peelably secured to one another; the device comprises: means defining an opening station for the said at least one container; means for peeling the members apart at the opening station to open the container; and an outlet, communicating with the opened container, through which a user can inhale the pharmaceutical composition in powder form from the opened container.

Preferably the composition is in unit dose form such as a tablet or capsule for oral administration, e.g. for oral administration to a human.

In the pharmaceutical composition, a or each dosage unit for oral or parenteral administration preferably contains from 0.01 to 3000 mg, more preferably 0.5 to 1000 mg, of a compound of the formula (I) or a pharmaceutically acceptable salt thereof, calculated as the free base. A or each dosage unit for nasal or inhaled administration preferably contains from 0.001 to 50 mg, more preferably 0.01 to 5 mg, of a compound of the formula (I) or a pharmaceutically acceptable salt thereof, calculated as the free base.

A pharmaceutically acceptable compound or salt of the invention is preferably administered to a mammal (e.g. human) in a daily oral or parenteral dose of 0.001 mg to 50 mg per kg body weight per day (mg/kg/day), for example 0.01 to 20 mg/kg/day or 0.03 to 10 mg/kg/day or 0.1 to 2 mg/kg/day, of the compound of the formula (I) or a pharmaceutically acceptable salt thereof, calculated as the free base.

A pharmaceutically acceptable compound or salt of the invention is preferably administered to a mammal (e.g. human) in a daily nasal or inhaled dose of: 0.0001 to 5 mg/kg/day or 0.0001 to 1 mg/kg/day, e.g. 0.001 to 1 mg/kg/day or 0.001 to 0.3 mg/kg/day or 0.001 to 0.1 mg/kg/day or 0.005 to 0.3 mg/kg/day, of the compound of the formula (I) or a pharmaceutically acceptable salt thereof, calculated as the free base.

The pharmaceutically acceptable compounds or salts of the invention is preferably administered in a daily dose (for an adult patient) of, for example, an oral or parenteral dose of 0.01 mg to 3000 mg per day or 0.5 to 1000 mg per day e.g. 2 to 500 mg per day, or a nasal or inhaled dose of 0.001 to 300 mg per day or 0.001 to 50 mg per day or 0.01 to 30 mg per day or 0.01 to 5 mg per day or 0.02 to 2 mg per day, of the compound of the formula (I) or a pharmaceutically acceptable salt thereof, calculated as the free base.

The compounds, salts and/or pharmaceutical compositions according to the invention may also be used in combination with another therapeutically active agent, for example, a β₂ adrenoreceptor agonist, an anti-histamine, an anti-allergic or an anti-inflammatory agent.

The invention thus provides, in a further aspect, a combination comprising a compound of formula (I) or a pharmaceutically acceptable salt thereof together with another therapeutically active agent, for example, a β₂-adrenoreceptor agonist, an anti-histamine, an anti-allergic, an anti-inflammatory agent or an antiinfective agent.

Preferably, the β₂-adrenoreceptor agonist is salmeterol (e.g. as racemate or a single enantiomer such as the R-enantiomer), salbutamol, formoterol, salmefamol, fenoterol or terbutaline, or a salt thereof (e.g. pharmaceutically acceptable salt thereof), for example the xinafoate salt of salmeterol, the sulphate salt or free base of salbutamol or the fumarate salt of formoterol. Long-acting β₂-adrenoreceptor agonists are preferred, especially those having a therapeutic effect over a 12-24 hour period such as salmeterol or formoterol. Preferably, the β₂-adrenoreceptor agonist is for inhaled administration, e.g. once per day and/or for simultaneous inhaled administration; and more preferably the β₂-adrenoreceptor agonist is in particle-size-reduced form e.g. as defined herein. Preferably, the β₂-adrenoreceptor agonist combination is for treatment and/or prophylaxis of COPD or asthma. Salmeterol or a pharmaceutically acceptable salt thereof, e.g. salmeterol xinofoate, is preferably administered to humans at an inhaled dose of 25 to 50 micrograms twice per day (measured as the free base). The combination with a β₂-adrenoreceptor agonist can be as described in WO 00/12078.

Preferred long acting β₂-adrenoreceptor agonists include those described in WO 02/066422A, WO 03/024439, WO 02/070490 and WO 02/076933.

Especially preferred long-acting β₂-adrenoreceptor agonists include compounds of formula (XX) (described in WO 02/066422): 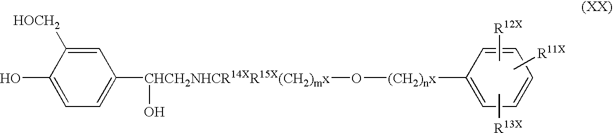
or a salt or solvate thereof, wherein in formula (XX):

m^{X} is an integer of from 2 to 8;

n^{X} is an integer of from 3 to 11,

with the proviso that m^{X}+n^{X} is 5 to 19,

R^{11X} is —XSO₂NR^{16X}R^{17X} wherein X is —(CH₂)ₚ^{x}— or C₂₋₆ alkenylene;

R^{16X} and R^{17X} are independently selected from hydrogen, C₁₋₆alkyl, C₃₋₇cycloalkyl, C(O)NR^{18X}R^{19X}, phenyl, and phenyl (C₁₋₄alkyl)-,

or R^{16X} and R^{17X}, together with the nitrogen to which they are bonded, form a 5-, 6-, or 7-membered nitrogen containing ring, and R^{16X} and R^{17X} are each optionally substituted by one or two groups selected from halo, C₁₋₆alkyl, C₁₋₆haloalkyl, C₁₋₆alkoxy, hydroxy-substituted C₁₋₆alkoxy, —CO₂R^{18X}, —SO₂NR^{18X}R^{19X}, —CONR^{18X}R^{19X}, —NR^{18X}C(O)R^{19X}, or a 5-, 6- or 7-membered heterocyclic ring;

R^{18X} and R^{19X} are independently selected from hydrogen, C₁₋₆alkyl, C₃₋₆cycloalkyl, phenyl, and phenyl (C₁₋₄alkyl)-; and

p^{X} is an integer of from 0 to 6, preferably from 0 to 4;

R^{12X} and R^{13X} are independently selected from hydrogen, C₁₋₆alkyl, C₁₋₆alkoxy, halo, phenyl, and C₁₋₆haloalkyl; and

R^{14X} and R^{15X} are independently selected from hydrogen and C₁₋₄alkyl with the proviso that the total number of carbon atoms in R^{14X} and R^{15X} is not more than 4.

Preferred β₂-adrenoreceptor agonists disclosed in WO 02/066422 include:

3-(4-{[6-({(2R)-2-hydroxy-2-[4-hydroxy-3-(hydroxymethyl)-phenyl]ethyl}amino)hexyl]oxy}butyl)benzenesulfonamide and 3-(3-{[7-({(2R)-2-hydroxy-2-[4-hydroxy-3-hydroxymethyl)phenyl]ethyl}-amino)heptyl]oxy}propyl)benzenesulfonamide.

A preferred β₂-adrenoreceptor agonist disclosed in WO 03/024439 is:

4-{(1R)-2-[(6-{2-[(2,6-dichlorobenzyl)oxy]ethoxy}hexyl)amino]-1-hydroxyethyl}-2-(hydroxymethyl)phenol.

A combination of a compound of formula (I) or salt together with an anti-histamine is preferably for oral administration (e.g. as a combined composition such as a combined tablet), and can be for treatment and/or prophylaxis of allergic rhinitis. Examples of anti-histamines include methapyrilene, or H1 antagonists such as cetirizine, loratadine (e.g. Clarityn™), desloratadine (e.g. Clarinex™) or fexofenadine (e.g. Allegra™).

The invention also provides, in a further aspect, a combination comprising a compound of formula (I) or a pharmaceutically acceptable salt thereof together with an anticholinergic compound, e.g. a muscarinic (M) receptor antagonist in particular an M₁, M₂, M₁/M₂, or M₃ receptor antagonist, more preferably a M₃ receptor antagonist, still more preferably a M₃ receptor antagonist which selectively antagonises (e.g. antagonises 10 times or more strongly) the M₃ receptor over the M₁ and/or M₂ receptor. For combinations of anticholinergic compounds/muscarinic (M) receptor antagonist with PDE4 inhibitors, see for example WO 03/011274 A2 and WO 02/069945 A2/US 2002/0193393 A1 and US 2002/052312 A1, and some or all of these publications give examples of anticholinergic compounds/muscarinic (M) receptor antagonists which may be used with the compounds of formula (I) or salts, and/or suitable pharmaceutical compositions. For example, the muscarinic receptor antagonist can comprise or be an ipratropium salt (e.g. ipratropium bromide), an oxitropium salt (e.g. oxitropium bromide), or more preferably a tiotropium salt (e.g. tiotropium bromide); see e.g. EP 418 716 A1 for tiotropium.

The anticholinergic compound or muscarinic (M) receptor antagonist, e.g. M₃ receptor antagonist, is preferably for inhaled administration, more preferably in particle-size-reduced form e.g. as defined herein. More preferably, both the muscarinic (M) receptor antagonist and the compound of formula (I) or the pharmaceutically acceptable salt thereof are for inhaled administration. Preferably, the anticholinergic compound or muscarinic receptor antagonist and the compound of formula (I) or salt are for simultaneous administration. The muscarinic receptor antagonist combination is preferably for treatment and/or prophylaxis of COPD.

Other suitable combinations include, for example, a combination comprising a compound of formula (I) or a pharmaceutically acceptable salt thereof together with another anti-inflammatory agent such as an anti-inflammatory corticosteroid; or a non-steroidal anti-inflammatory drug (NSAID) such as a leukotriene antagonist (e.g. montelukast), an iNOS inhibitor, a tryptase inhibitor, a elastase inhibitor, a beta-2 integrin antagonist, a adenosine 2a agonist, a CCR3 antagonist, or a 5-lipoxogenase inhibitor; or an antiinfective agent (e.g. an antibiotic or an antiviral). An iNOS inhibitor is preferably for oral administration. Suitable iNOS inhibitors (inducible nitric oxide synthase inhibitors) include those disclosed in WO 93/13055, WO 98/30537, WO 02/50021, WO 95/34534 and WO 99/62875. Suitable CCR3 inhibitors include those disclosed in WO 02/26722.

In a combination comprising a compound of formula (I) or a pharmaceutically acceptable salt thereof together with an anti-inflammatory corticosteroid (which is preferably for treatment and/or prophylaxis of asthma, COPD or allergic rhinitis), then preferably the anti-inflammatory corticosteroid is fluticasone, fluticasone propionate (e.g. see U.S. Pat. No. 4,335,121), beclomethasone, beclomethasone 17-propionate ester, beclomethasone 17,21-dipropionate ester, dexamethasone or an ester thereof, mometasone or an ester thereof, ciclesonide, budesonide, flunisolide, or a compound as described in WO 02/12266 A1 (e.g. as claimed in any of claims 1 to 22 therein), or a pharmaceutically acceptable salt of any of the above. If the anti-inflammatory corticosteroid is a compound as described in WO 02/12266 A1, then preferably it is Example 1 therein {which is 6α,9α-difluoro-17α-[(2-furanylcarbonyl)oxy]-11β-hydroxy-16α-methyl-3-oxo-androsta-1,4-diene-17β-carbothioic acid S-fluoromethyl ester} or Example 41 therein {which is 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester}, or a pharmaceutically acceptable salt thereof. The anti-inflammatory corticosteroid is preferably for intranasal or inhaled administration. Fluticasone propionate is preferred and is preferably for inhaled administration to a human either (a) at a dose of 250 micrograms once per day or (b) at a dose of 50 to 250 micrograms twice per day.

Also provided is a combination comprising a compound of formula (I) or a pharmaceutically acceptable salt thereof together with β₂-adrenoreceptor agonist and an anti-inflammatory corticosteroid, for example as described in WO 03/030939 A1. Preferably this combination is for treatment and/or prophylaxis of asthma, COPD or allergic rhinitis. The β₂-adrenoreceptor agonist and/or the anti-inflammatory corticosteroid can be as described above and/or as described in WO 03/030939 A1. Most preferably, in this “triple” combination, the β₂-adrenoreceptor agonist is salmeterol or a pharmaceutically acceptable salt thereof (e.g. salmeterol xinafoate) and the anti-inflammatory corticosteroid is fluticasone propionate.

The combinations referred to above may conveniently be presented for use in the form of a pharmaceutical composition and thus a pharmaceutical composition comprising a combination as defined above together with one or more pharmaceutically acceptable carriers and/or excipients represent a further aspect of the invention.

The individual compounds of such combinations may be administered either sequentially or simultaneously in separate or combined pharmaceutical composition.

In one embodiment, the combination as defined herein can be for simultaneous inhaled administration and is disposed in a combination inhalation device. Such a combination inhalation device is another aspect of the invention. Such a combination inhalation device can comprise a combined pharmaceutical composition for simultaneous inhaled administration (e.g. dry powder composition), the composition comprising all the individual compounds of the combination, and the composition being incorporated into a plurality of sealed dose containers mounted longitudinally in a strip or ribbon inside the inhalation device, the containers being rupturable or peel-openable on demand; for example such inhalation device can be substantially as described in GB 2,242,134 A (DISKUS™) and/or as described above. Alternatively, the combination inhalation device can be such that the individual compounds of the combination are administrable simultaneously but are stored separately (or wholly or partly stored separately for triple combinations), e.g. in separate pharmaceutical compositions, for example as described in PCT/EP03/00598 filed on 22 Jan. 2003, published as WO 03/061743 (e.g. as described in the claims thereof e.g. claim 1).

The invention also provides a method of preparing a combination as defined herein,
the method comprising either
(a) preparing a separate pharmaceutical composition for administration of the individual compounds of the combination either sequentially or simultaneously, or
(b) preparing a combined pharmaceutical composition for administration of the individual compounds of the combination simultaneously,
wherein the pharmaceutical composition comprises the combination together with one or more pharmaceutically acceptable carriers and/or excipients.

The invention also provides a combination as defined herein, prepared by a method as defined herein.

Biological Test Methods

PDE 3, PDE 4B, PDE 4D, PDE 5, PDE 6 Primary Assay Methods

The activity of the compounds can be measured in the assay methods shown below. Preferred compounds of the invention are selective PDE4 inhibitors, i.e. they inhibit PDE4 (e.g. PDE4B and/or PDE4D, preferably PDE4B) more strongly than they inhibit PDE3 and/or more strongly than they inhibit PDE5 and/or more strongly than they inhibit PDE6.

PDE Enzyme Sources and Literature References

Human recombinant PDE4B, in particular the 2B splice variant thereof (HSPDE4B2B), is disclosed in WO 94/20079 and also M. M. McLaughlin et al., “A low Km, rolipram-sensitive, cAMP-specific phosphodiesterase from human brain: cloning and expression of cDNA, biochemical characterisation of recombinant protein, and tissue distribution of mRNA”, J. Biol. Chem., 1993, 268, 6470-6476. For example, in Example 1 of WO 94/20079, human recombinant PDE4B is described as being expressed in the PDE-deficient yeast Saccharomyces cerevisiae strain GL62, e.g. after induction by addition of 150 uM CuSO₄, and 100,000×g supernatant fractions of yeast cell lysates are described for use in the harvesting of PDE4B enzyme.

Human recombinant PDE4D (HSPDE4D3A) is disclosed in P. A. Baecker et al., “Isolation of a cDNA encoding a human rolipram-sensitive cyclic AMP phoshodiesterase (PDE IV_{D})”, Gene, 1994, 138, 253-256.

Human recombinant PDE5 is disclosed in K. Loughney et al., “Isolation and characterisation of cDNAs encoding PDE5A, a human cGMP-binding, cGMP-specific 3′,5′-cyclic nucleotide phosphodiesterase”, Gene, 1998, 216, 139-147.

PDE3 can be purified from bovine aorta, e.g. as described by H. Coste and P. Grondin, “Characterisation of a novel potent and specific inhibitor of type V phosphodiesterase”, Biochem. Pharmacol., 1995, 50, 1577-1585.

PDE6 can be purified from bovine retina, e.g. as described by: P. Catty and P. Deterre, “Activation and solubilization of the retinal cGMP-specific phosphodiesterase by limited proteolysis”, Eur. J. Biochem., 1991, 199, 263-269; A. Tar et al. “Purification of bovine retinal cGMP phosphodiesterase”, Methods in Enzymology, 1994, 238, 3-12; and/or D. Srivastava et al. “Effects of magnesium on cyclic GMP hydrolysis by the bovine retinal rod cyclic GMP phosphodiesterase”, Biochem. J., 1995, 308, 653-658.

Inhibition of PDE 3, PDE 4B, PDE 4D, PDE 5 or PDE 6 Activity: Radioactive Scintillation Proximity Assay (SPA)

The ability of compounds to inhibit catalytic activity at PDE4B or 4D (human recombinant), PDE3 (from bovine aorta), PDE5 (human recombinant) or PDE6 (from bovine retina) is determined by Scintillation Proximity Assay (SPA) in 96-well format. Test compounds (as a solution in DMSO, preferably about 2 microlitre (ul) volume of DMSO solution) are preincubated at ambient temperature (room temperature, e.g. 19-23° C.) in Wallac Isoplates (code 1450-514) with PDE enzyme in 50 mM Tris-HCl buffer pH 7.5, 8.3 mM MgCl₂, 1.7 mM EGTA, 0.05% (w/v) bovine serum albumin for 10-30 minutes (usually 30 minutes). The enzyme concentration is adjusted so that no more than 20% hydrolysis of the substrate defined below occurs in control wells without compound, during the incubation. For the PDE3, PDE4B and PDE4D assays, [5′,8-³H]Adenosine 3′,5′-cyclic phosphate (Amersham Pharmacia Biotech, code TRK.559; or Amersham Biosciences UK Ltd, Pollards Wood, Chalfont St Giles, Buckinghamshire HP8 4SP, UK) is added to give 0.05 uCi per well and ˜10 nM final concentration. For the PDE5 and PDE6 assays, [8-³H]Guanosine 3′,5′-cyclic phosphate (Amersham Pharmacia Biotech, code TRK.392) is added to give 0.05 uCi per well and ˜36 nM final concentration. Plates containing assay mixture, preferably approx. 100 ul volume of assay mixture, are mixed on an orbital shaker for 5 minutes and incubated at ambient temperature for 1 hour. Phosphodiesterase SPA beads (Amersham Pharmacia Biotech, code RPNQ 0150) are added (˜1 mg per well) to terminate the assay. Plates are sealed and shaken and allowed to stand at ambient temperature for 35 minutes to 1 hour (preferably 35 minutes) to allow the beads to settle. Bound radioactive product is measured using a WALLAC TRILUX 1450 Microbeta scintillation counter. For inhibition curves, 10 concentrations (1.5 nM -30 uM) of each compound are assayed. Curves are analysed using ActivityBase and XLfit (ID Business Solutions Limited, 2 Ocean Court, Surrey Research Park, Guildford, Surrey GU2 7QB, United Kingdom) Results are expressed as pIC₅₀ values.

In an alternative to the above radioactive SPA assay, PDE4B or PDE4D inhibition can be measured in the following Fluorescence Polarisation (FP) assay:

Inhibition of PDE4B or PDE4D Activity: Fluorescence Polarisation (FP) Assay

The ability of compounds to inhibit catalytic activity at PDE4B (human recombinant) or PDE4D (human recombinant) is determined by IMAP Fluorescence Polarisation (FP) assay (IMAP Explorer kit, available from Molecular Devices Corporation, Sunnydale, Calif., USA; Molecular Devices code: R8062) in 384-well format. The IMAP FP assay is able to measure PDE activity in an homogenous, non-radioactive assay format. The FP assay uses the ability of immobilised trivalent metal cations, coated onto nanoparticles (tiny beads), to bind the phosphate group of Fl-AMP that is produced on the hydrolysis of fluorescein-labelled (Fl) cyclic adenosine mono-phosphate (Fl-cAMP) to the non-cyclic Fl-AMP form. Fl-cAMP does not bind. Binding of Fl-AMP product to the beads (coated with the immobilised trivalent cations) slows the rotation of the bound Fl-AMP and leads to an increase in the fluorescence polarisation ratio of parallel to perpendicular light. Inhibition of the PDE reduces/inhibits this signal increase.

Test compounds (small volume, e.g. ca. 0.5 to 1 ul, preferably ca. 0.5 ul, of solution in DMSO) are preincubated at ambient temperature (room temperature, e.g. 19-23° C.) in black 384-well microtitre plates (supplier: NUNC, code 262260) with PDE enzyme in 10 mM Tris-HCl buffer pH 7.2, 11 mM MgCl₂, 0.1% (w/v) bovine serum albumin, and 0.05% NaN₃ for 10-30 minutes. The enzyme level is set by experimentation so that reaction is linear throughout the incubation. Fluorescein adenosine 3′,5′-cyclic phosphate (from Molecular Devices Corporation, Molecular Devices code: R7091) is added to give about 40 nM final concentration (final assay volume usually ca. 20-40 ul, preferably ca. 20 ul). Plates are mixed on an orbital shaker for 10 seconds and incubated at ambient temperature for 40 minutes. IMAP binding reagent (as described above, from Molecular Devices Corporation, Molecular Devices code: R7207) is added (60 ul of a 1 in 400 dilution in binding buffer of the kit stock solution) to terminate the assay. Plates are allowed to stand at ambient temperature for 1 hour. The Fluorescence Polarisation (FP) ratio of parallel to perpendicular light is measured using an Analyst™ plate reader (from Molecular Devices Corporation). For inhibition curves, 10 concentrations (1.5 nM -30 uM) of each compound are assayed. Curves are analysed using ActivityBase and XLfit (ID Business Solutions Limited, 2 Ocean Court, Surrey Research Park, Guildford, Surrey GU2 7QB, United Kingdom). Results are expressed as pIC₅₀ values.

In the FP assay, all reagents are dispensed using Multidrop™ (available from Thermo Labsystems Oy, Ratastie 2, PO Box 100, Vantaa 01620, Finland).

For a given PDE4 inhibitor, the PDE4B (or PDE4D) inhibition values measured using the SPA and FP assays can differ slightly. However, in a regression analysis of 100 test compounds (not necessarily compounds of the invention), the pIC₅₀ inhibition values measured using SPA and FP assays have been found generally to agree within 0.5 log units, for PDE4B and PDE4D (linear regression coefficient 0.966 for PDE4B and 0.971 for PDE4D; David R. Mobbs et al., “Comparison of the IMAP Fluorescence Polarisation Assay with the Scintillation Proximity Assay for Phosphodiesterase Activity”, poster presented at 2003 Molecular Devices UK & Europe User Meeting, 2 Oct. 2003, Down Hall, Harlow, Essex, United Kingdom).

Biological Data obtained for some of the Examples (PDE4B inhibitory activity, either as one reading or as an average of ca. 2-6 readings) are as follows, based on current measurements only. In each of the SPA and FP assays, absolute accuracy of measurement is not possible, and the readings given are accurate only up to about ±0.5 of a log unit, depending on the number of readings made and averaged:

All of the Examples have been tested for PDE4B inhibition using the radioactive SPA assay or the FP assay or in a similar assay. All of the Examples tested have PDE4B inhibitory activities in the range of pIC₅₀=about 7 (±about 0.5) to about 9 (±about 0.5).

All publications, including but not limited to patents and patent applications, cited in this specification are herein incorporated by reference as if each individual publication were specifically and individually indicated to be incorporated by reference herein as though fully set forth.

### EXAMPLES

The various aspects of the invention will now be described by reference to the following examples. These examples are merely illustrative and are not to be construed as a limitation of the scope of the present invention. In this section, “Intermediates” represent syntheses of intermediate compounds intended for use in the synthesis of the “Examples”.

Abbreviations Used Herein:

CCl₄ carbon tetrachloride
DCM dichloromethane
DMF dimethyl formamide
DIPEA diisopropylethyl amine (ⁱPr₂NEt)
EtOAc ethyl acetate
Et₂O diethyl ether
Et₃N triethylamine
EtOH ethanol
HATU O-(7-azabenzotriazol-1-yl)-N,N,N′,N′-tetramethyluronium hexafluorophosphate
HCl hydrogen chloride/hydrochloric acid
Na₂SO₄ sodium sulfate
NaHCO₃ sodium bicarbonate
NMP 1-methyl-2-pyrrolidinone
PhCH₃ toluene
PPA polyphosphoric acid
HPLC high pressure liquid chromatography
SPE solid phase extraction
NMR nuclear magnetic resonance (in which: s=singlet, d=doublet, t=triplet, q=quartet, dd=doublet of doublets, m=multiplet, H=no. of protons)
LCMS liquid chromatography/mass spectroscopy
TLC thin layer chromatography
h hours
T_{RET} retention time
Room temperature this is usually in the range of about 20 to about 25° C.
   General Experimental Details
   Machine Methods Used Herein:
   LCMS (Liquid Chromatography/Mass Spectroscopy)
Waters ZQ mass spectrometer operating in positive ion electrospray mode, mass range 100-1000 amu.
UV wavelength: 215-330 nM
Column: 3.3cm×4.6mm ID, 3 μm ABZ+PLUS
Flow Rate: 3 ml/min
Injection Volume: 5 μl
Solvent A: 95% acetonitrile+0.05% formic acid
Solvent B: 0.1% formic acid+10 mMolar ainmonium acetate
Gradient: 0% A/0.7 min, 0-100% A/3.5 min, 100% A/1.1 min, 100-0% A/0.2 min

It should be noted that retention times (T_{RET}) quoted herein may vary slightly (±0.1 min.) when samples were run on different Waters machines, even though the same type of column and identical flow rates, injection volumes, solvents and gradients were used.

Mass Directed Autoprep HPLC

The prep column used was a Supelcosil ABZplus (10 cm×2.12 cm) (usually 10 cm×2.12 cm×5 μm).
UV wavelength: 200-320 nM
Flow: 20 ml/min
Injection Volume: 1 ml; or more preferably 0.5 ml
Solvent A: 0.1% formic acid
Solvent B: 95% acetonitrile+5% formic acid; or more usually 99.95% acetonitrile+0.05% formic acid
Gradient: 100% A/1 min, 100-80% A/9 min, 80-1% A/3.5 min, 1% A/1.4 min, 1-100% A/0.1 min

### Intermediates and Examples

All reagents not detailed in the text below are corumercially available from established suppliers such as Sigma-Aldrich. The addresses of the suppliers for some of the starting materials mentioned in the Intermediates and Examples below or the Assays above are as follows:
Aldrich (catalogue name), Sigma-Aldrich Company Ltd., Dorset, United Kingdom, telephone: +44 1202 733114; Fax: +44 1202 715460; ukcustsv@eurnotes.sial.com; or
Aldrich (catalogue name), Sigma-Aldrich Corp., P.O. Box 14508, St. Louis, Mo. 63178-9916, USA; telephone: 314-771-5765; fax: 314-771-5757; custserv@sial.com; or
Aldrich (catalogue name), Sigma-Aldrich Chemie Gmbh, Munich, Germany; telephone: +49 89 6513 0; Fax: +49 89 6513 1169; deorders@eurnotes.sial.com.
Bionet Research Ltd; Highfield Industrial Estate, Camelford, Cornwall PL32 9QZ UK
Combi-Blocks Inc., 7949 Silverton Avenue, Suite 915, San Diego, Calif. 92126, USA (CAS 38041-19-9)
Lancaster Synthesis Ltd., Newgate, White Lund, Morecambe, Lancashire LA3 3DY, United Kingdom
Matrix Scientific, P.O. Box 25067, Columbia, S.C. 29224-5067, USA

Trans World Chemicals, Inc., 14674 Southlawn Lane, Rockville, Md. 20850, USA

Table of Intermediates

| Inter- | |
|---|---|
| mediate | |
| Number | Name |
| 1 | Diethyl (cyclopropylcarbonyl)malonate |
| 2 | Diethyl (cyclopentylcarbonyl) malonate |
| 3 | Diethyl (propylcarbonyl)malonate |
| 4 | Diethyl (ethylcarbonyl)malonate |
| 5 | Diethyl (cyclobutylcarbonyl)malonate |
| 6 | Diethyl (cyclopropylmethylcarbonyl)malonate |
| 7 | Diethyl [chloro(cyclopropyl)methylene]malonate |
| 8 | Diethyl [chloro(propyl)methylene] malonate |
| 9 | Diethyl [chloro(ethyl)methylene] malonate |
| 10 | Diethyl [chloro(cyclobutyl)methylene] malonate |
| 11 | Diethyl [chloro(cyclopropylmethyl)methylene] malonate |
| 12 | Ethyl 4-chloro-6-cyclopropyl-1-ethyl-1H-pyrazolo[3,4-b]pyridine-5- |
| | carboxylate |
| 13 | Ethyl-4-chloro-6-propyl-1-ethyl-1H-pyrazolo[3,4-b]pyridine-5-carboxylate |
| 14 | Ethyl-4-chloro-6-ethyl-1-ethyl-1H-pyrazolo[3,4-b]pyridine-5-carboxylate |
| 15 | Ethyl-4-chloro-6-cyclobutyl-1-ethyl-1H-pyrazolo[3,4-b]pyridine-5- |
| | carboxylate |
| 16 | Ethyl-4-chloro-6-cyclopropylmethyl-1-ethyl-1H-pyrazolo[3,4-b]pyridine-5- |
| | carboxylate |
| 17 | Ethyl-4-chloro-6-cyclopentyl-1-ethyl-1H-pyrazolo[3,4-b]pyridine-5- |
| | carboxylate |
| 18 | 4-Aminotetrahydropyran |
| 19 | Ethyl 6-cyclopropyl-1-ethyl-4-(tetrahydro-2H-pyran-4-ylamino)-1H- |
| | pyrazolo[3,4-b]pyridine-5-carboxylate |
| 20 | Ethyl 6-propyl- 1-ethy1-4-(tetrahydro-2H-pyran-4-ylamino)-1H- |
| | pyrazolo[3,4-b]pyridine-5-carboxylate |
| 21 | Ethyl 6-ethyl-1-ethyl-4-(tetrahydro-2H-pyran-4-ylamino)-1H-pyrazolo[3,4- |
| | b]pyridine-5-carboxylate |
| 22 | Ethyl 6-cyclobutyl-1-ethyl-4-(tetrahydro-2H-pyran-4-ylamino)-1H- |
| | pyrazolo[3,4-b]pyridine-5-carboxylate |
| 23 | Ethyl 6-cyclopropylmethyl-1-ethyl-4-(tetrahydro-2H-pyran-4-ylamino)-1H- |
| | pyrazolo[3,4-b]pyridine-5-carboxylate |
| 24 | Ethyl 6-cyclopentyl-1-ethyl-4-(tetrahydro-2H-pyran-4-ylamino)-1H- |
| | pyrazolo[3,4-b]pyridine-5-carboxylate |
| 25 | 6-Cyclopropyl-1-ethyl-4-(tetrahydro-2H-pyran-4-ylamino)-1H-pyrazolo[3,4- |
| | b]pyridine-5-carboxylic acid |
| 26 | 6-propyl- 1-ethyl-4-(tetrahydro-2H-pyran-4-ylamino)-1H-pyrazolo[3,4- |
| | b]pyridine-5-carboxylic acid |
| 27 | 6-Ethyl-1-ethyl-4-(tetrahydro-2H-pyran-4-ylamino)-1H-pyrazolo[3,4- |
| | b]pyridine-5-carboxylic acid |
| 28 | 6-Cyclobutyl-1-ethyl-4-(tetrahydro-2H-pyran-4-ylamino)-1H-pyrazolo[3,4- |
| | 6]pyridine-5-carboxylic acid |
| 29 | 6-Cyclopropylmethyl-1-ethyl-4-(tetrahydro-2H-pyran-4-ylamino)-1H- |
| | pyrazolo[3,4-b]pyridine-5-carboxylic acid |
| 30 | 6-Cyclopentyl-1-ethyl-4-(tetrahydro-2H-pyran-4-ylamino)-1H-pyrazolo[3,4- |
| | b]pyridine-5-carboxylic acid |

### Cyclopropylacetyl Chloride

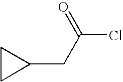

Prepared from cyclopropyl acetic acid according to the procedure outlined in: Bouzoubaa, Mohamed; Leclerc, Gerard; Decker, Nicole; Schwartz, Jean; Andermann, Guy; J. Med. Chem; 1984, 27 (10); 1291-1294.

### Intermediate 1: Diethyl(cyclopropylcarbonyl)malonate

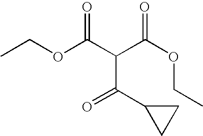

A solution of diethyl malonate (6 ml) in EtOH (20 ml) was added to solid magnesium powder (1 g) and CCl₄ (1 ml) at such a rate as to maintain a constant reflux. Et₂O (20 ml) was added and the mixture was heated under reflux for 2 h. The solvents were removed under reduced pressure and the residue was azeotroped with PhCH₃ (2×10 ml). The solid residue was dissolved in Et₂O (40 ml) and the solution was added to a cooled solution of cylopropane carbonyl chloride (3.6 ml), maintaining the temperature between 0 and 5° C. The mixture was allowed to warm slowly to room temperature and stirred for 16 hours. The mixture was treated with 5% aqueous sulphuric acid solution (100 ml), with stirring. The organic phase was separated, washed with saturated aqueous NaHCO₃ (2×75 ml), dried over anhydrous Na₂SO₄, filtered and the filtrate evaporated to dryness to leave Intermediate 1 as a colourless oil (7.74 g). LCMS showed MH⁺=229; T_{RET}=2.79 min.

Similarly prepared from diethyl malonate were the following:

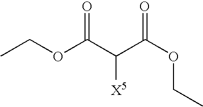

| | | MH⁺ | T_{RET} |
|---|---|---|---|
| | X⁵ | ion | (min) |
| Intermediate 2 | 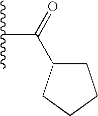 | 257 | 3.2 |
| Intermediate 3 | 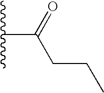 | 231 | 3.09 |
| Intermediate 4 | 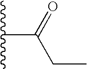 | 217 | 2.81 |
| Intermediate 5 | 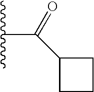 | 243 | 3.07 |
| Intermediate 6 | 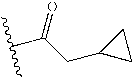 | 243 | 3.07 |

### Intermediates 7-11

These intermediates were prepared using a modification of the procedure developed by O. E. O. Hormi and described in Synthetic Commun; 1986, 16 997-1002.

### Intermediate 7: Diethyl[chloro(cyclopropyl)methylene]malonate

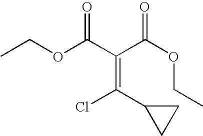

A mixture of Intermediate 1 (1 g), phosphorus oxychloride (10 ml) and tributylamine (1.1 ml) was stirred at 110° C. for 5 h. The volatile organics were removed under reduced pressure. The mixture was dissolved in Et₂O (20 ml) and n-hexane was added until two layers formed. The top layer was collected. More n-hexane was added until two layers formed again. This procedure was repeated once more. The combined Et₂O extracts were washed with dilute aqueous HCl (1M, 2×50 ml), dilute aqueous NaOH (1M, 2×50 ml) and water (2×50 ml), dried over anhydrous Na₂SO₄, filtered and evaporated to give Intermediate 7 as a pale yellow oil (0.87 g). LCMS showed MH⁺=249; T_{RET}=3.49 min.

Similarly prepared from Intermediates 3-6 were the following:

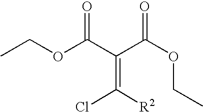

| | | | | T_{RET} |
|---|---|---|---|---|
| | Precursor | R² | MH⁺ ion | (min) |
| Intermediate 8 | Intermediate 3 | 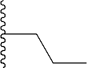 | 249 | 3.45 |
| Intermediate 9 | Intermediate 4 | 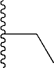 | 235 | 3.21 |
| Intermediate 10 | Intermediate 5 | 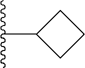 | 261 | 3.44 |
| Intermediate 11 | Intermediate 6 | 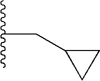 | 261 | 3.23 |

### Intermediate 12: Ethyl 4-chloro-6-cyclopropyl-1-ethyl-1H-pyrazolo[3,4-b]pyridine-5-carboxylate

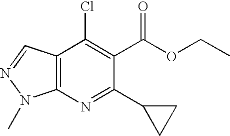

A mixture of intermediate 7 (6.75 g), Et₃N (2.88 ml) and 5-amino-1-ethyl pyrazole (3.28 g) in PhCH₃ (40 ml) was heated at reflux for 16 hours. The solvent was removed under reduced pressure and the residue was treated with phosphorus oxychloride (50 ml). The reaction mixture was heated at 110° C. for 16 hours. The phosphorus oxychloride was removed under reduced pressure and the residue was partitioned between EtOAc (50 ml) and saturated aqueous NaHCO₃ (50 ml). The organic layer was collected, dried (Na₂SO₄), filtered and the filtrate was concentrated under reduced pressure. The residue was purified on a 50 g SiO₂ SPE cartridge, eluting with 5% EtOAc: 95% cyclohexane, to give Intermediate 12 as a yellow oil (1.53 g). LCMS showed MH⁺=294; T_{RET}=3.4 min.

Similarly prepared from Intermediates 8-11 were the following:

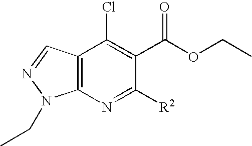

| | | | | T_{RET} |
|---|---|---|---|---|
| | Precursor | R² | MH⁺ ion | (min) |
| Intermediate 13 | Intermediate 8 | 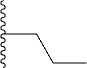 | 296 | 3.63 |
| Intermediate 14 | Intermediate 9 | 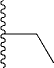 | 282 | 3.4 |
| Intermediate 15 | Intermediate 10 | 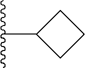 | 308 | 3.71 |
| Intermediate 16 | Intermediate 11 | 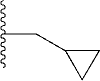 | 308 | 3.71 |

### Intermediate 17: Ethyl-4-chloro-6-cyclopentyl-1-ethyl-1H-pyrazolo[3,4-b]pyridine-5-carboxylate

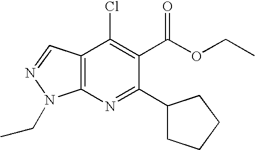

A mixture of Intermediate 2 (1.6 g) and 5-amino-i-ethyl pyrazole (0.69 g) in PPA (12 ml) was heated at 120° C. for 3 h. The mixture was poured into water (50 ml) and extracted with Et₂O (3×75 ml). The organic extracts were combined, dried (Na₂SO₄), filtered and the filtrate concentrated to leave a yellow oil. Phosphorus oxychloride (50 ml) was added to the oil. The mixture was heated at 110° for 16 h. The phosphorus oxychloride was removed and the residue was partitioned between EtOAc (50 ml) and saturated aqueous NaHCO₃ (25 ml). The organic layer was collected, dried (Na₂SO₄), filtered and the filtrate was concentrated to leave Intermediate 17 as a tan solid (100 mg). LCMS showed MH⁺=322; T_{RET}=3.96 min.

### Intermediate 18: 4-Aminotetrahydropyran

Commercially available from Combi-Blocks Inc., 7949 Silverton Avenue, Suite 915, San Diego, Calif. 92126, USA (CAS 38041-19-9)

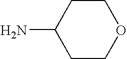

### Intermediate 19: Ethyl 6-cyclopropyl-1-ethyl-4-(tetrahydro-2H-pyran-4-ylamino)-1H-pyrazolo[3,4-b]pyridine-5-carboxylate

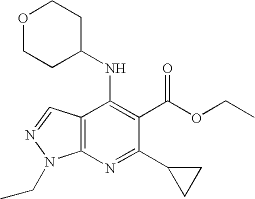

A solution of Intermediate 12 (0.76 g), Intermediate 18 (0.36 g) and DIPEA (0.65 ml) in NMP (8 ml) was heated at 150° C. for 16 h. The mixture was allowed to cool and partitioned between EtOAc (100 ml) and water (20 ml). The organic layer was collected, dried (Na₂SO₄), filtered and concentrated to give a dark gum. The gum was purified on a 20 g SiO₂ SPE cartridge, eluting with 10% EtOAc:90% Cyclohexane to give Intermediate 18 as a pale yellow solid (0.35 g). LCMS showed MH⁺=359; T_{RET}=3.43 min.

Similarly prepared from Intermediates 13-17 were the following:

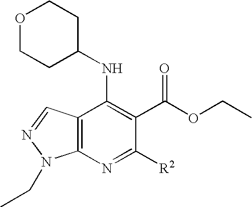

| | | | | T_{RET} |
|---|---|---|---|---|
| | Precursor | R² | MH⁺ ion | (min) |
| Intermediate 20 | Intermediate 13 | 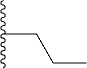 | 361 | 3.16 |
| Intermediate 21 | Intermediate 14 | 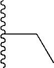 | 347 | 3.05 |
| Intermediate 22 | Intermediate 15 | 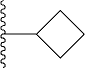 | 373 | 3.57 |
| Intermediate 23 | Intermediate 16 | 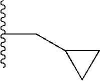 | 373 | 3.35 |
| Intermediate 24 | Intermediate 17 | 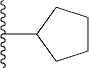 | 387 | 3.78 |

### Intermediate 25: 6-Cyclopropyl-1-ethyl-4-(tetrahydro-2H-pyran-4-ylamino)-1H-pyrazolo[3,4-b]pyridine-5-carboxylic Acid

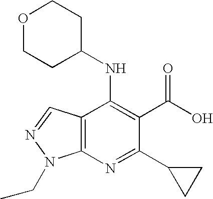

A solution of intermediate 19 (0.35 g) in EtOH (60 ml) was treated with a solution of NaOH (0.75 g) in water (20 ml) and the reaction mixture was heated at 60° C. for 16 h. The solvents were removed under reduced pressure, the residue was suspended in water (5 ml) and the pH of the solution was adjusted to 3 (1M HCl). The resultant suspension was extracted with EtOAc (2×30 ml). The combined organic extracts were dried (Na₂SO₄), filtered and concentrated under reduced pressure to give Intermediate 25 as a white solid (0.30 g). LCMS showed MH⁺=331; T_{RET}=2.41 min

Similarly prepared from Intermediates 21-25 were the following:

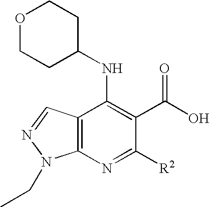

| | | | | T_{RET} |
|---|---|---|---|---|
| | Precursor | R² | MH⁺ ion | (min) |
| Intermediate 26 | Intermediate 21 | 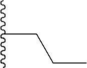 | 333 | 2.06 |
| Intermediate 27 | Intermediate 22 | 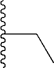 | 319 | 1.9 |
| Intermediate 28 | Intermediate 23 | 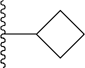 | 345 | 2.61 |
| Intermediate 29 | Intermediate 24 | 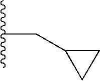 | 345 | 2.12 |
| Intermediate 30 | Intermediate 25 | 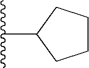 | 359 | 2.72 |

### Table of Examples

| Example | |
|---|---|
| Number | Name |
| 1 | N-[(4-chloro-2-methylphenyl)methyl]-6-cyclopropyl-1-ethyl-4-(tetrahydro- |
| | 2H-pyran-4-ylamino)-1H-pyrazolo[3,4-b]pyridine-5-carboxamide |
| 2 | 6-cyclopropyl-1-ethyl-N-(phenylmethyl)-4-(tetrahydro-2H-pyran-4- |
| | ylamino)-1H-pyrazolo[3,4-b]pyridine-5-carboxamide |
| 3 | 6-cyclopropyl-1-ethyl-N-{[4-(methyloxy)phenyl]methyl}-4-(tetrahydro-2H- |
| | pyran-4-ylamino)-1H-pyrazolo[3,4-b]pyridine-5-carboxamide |
| 4 | 6-cyclopropyl-N-[(3,4-dimethylphenyl)methyl]-1-ethyl-4-(tetrahydro-2H- |
| | pyran-4-ylamino)-1H-pyrazolo[3,4-b]pyridine-5-carboxamide |
| 5 | N-[1-(4-chlorophenyl)ethyl]-6-cyclopropyl-1-ethyl-4-(tetrahydro-2H-pyran- |
| | 4-ylamino)-1H-pyrazolo[3,4-b]pyridine-5-carboxamide |
| 6 | N-[1-(4-chlorophenyl)propyl]-6-cyclopropyl-1-ethyl-4-(tetrahydro-2H- |
| | pyran-4-ylamino)-1H-pyrazolo[3,4-b]pyridine-5-carboxamide |
| 7 | 1-ethyl-N-(phenylmethyl)-6-propyl-4-(tetrahydro-2H-pyran-4-ylamino)- |
| | 1H-pyrazolo[3,4-b]pyridine-5-carboxamide |
| 8 | 1-ethyl-N-{[4-(methyloxy)phenyl]methyl}-6-propyl-4-(tetrahydro-2H- |
| | pyran-4-ylamino)-1H-pyrazolo[3,4-b]pyridine-5-carboxamide |
| 9 | N-[(4-chloro-2-methylphenyl)methyl]-1-ethyl-6-propyl-4-(tetrahydro-2H- |
| | pyran-4-ylamino)-1H-pyrazolo[3,4-b]pyridine-5-carboxamide |
| 10 | N-[(3,4-dimethylphenyl)methyl]-1-ethyl-6-propyl-4-(tetrahydro-2H-pyran- |
| | 4-ylamino)-1H-pyrazolo[3,4-b]pyridine-5-carboxamide |
| 11 | N-(2,3-dihydro-1H-inden-2-yl)-1-ethyl-6-propyl-4-(tetrahydro-2H-pyran-4- |
| | ylamino)-1H-pyrazolo[3,4-b]pyridine-5-carboxamide |
| 12 | 1,6-diethyl-N-(phenylmethyl)-4-(tetrahydro-2H-pyran-4-ylamino)-1H- |
| | pyrazolo[3,4-b]pyridine-5-carboxamide |
| 13 | 1,6-diethyl-N-{[4-(methyloxy)phenyl]methyl}-4-(tetrahydro-2H-pyran-4- |
| | ylamino)-1H-pyrazolo[3,4-b]pyridine-5-carboxamide |
| 14 | N-[(3,4-dimethylphenyl)methyl]-1,6-dithyl-4-(tetrahydro-2H-pyran-4- |
| | ylamino)-1H-pyrazolo[3,4-b]pyridine-5-carboxamide |
| 15 | N-(2,3-dihydro-1H-inden-2-yl)-1,6-diethyl-4-(tetrahydro-2H-pyran-4- |
| | ylamino)-1H-pyrazolo[3,4-b]pyridine-5-carboxamide |
| 16 | N-[1-(4-chlorophenyl)propyl]-1,6-diethyl-4-(tetrahydro-2H-pyran-4- |
| | ylamino)-1H-pyrazolo[3,4-b]pyridine-5-carboxamide |
| 17 | 6-cyclobutyl-1-ethyl-N-(phenylmethyl)-4-(tetrahydro-2H-pyran-4- |
| | ylamino)-1H-pyrazolo[3,4-b]pyridine-5-carboxamide |
| 18 | 6-cyclobutyl-1-ethyl-N-{[4-(methyloxy)phenyl]methyl}-4-(tetrahydro-2H- |
| | pyran-4-ylamino)-1H-pyrazolo[3,4-b]pyridine-5-carboxamide |
| 19 | 6-(cyclopropylmethyl)-N-[(3,4-dimethylphenyl)methyl]-1-ethyl-4- |
| | (tetrahydro-2H-pyran-4-ylamino)-1H-pyrazolo[3,4-b]pyridine-5- |
| | carboxamide |
| 20 | 6-cyclobutyl-N-(2,3-dihydro-1H-inden-2-yl)-1-ethyl-4-(tetrahydro-2H- |
| | pyran-4-ylamino)-1H-pyrazolo[3,4-b]pyridine-5-carboxamide |
| 21 | N-[(4-chloro-2-methylphenyl)methyl]-6-cyclobutyl-1-ethyl-4-(tetrahydro- |
| | 2H-pyran-4-ylamino)-1H-pyrazolo[3,4-b]pyridine-5-carboxamide |
| 22 | N-[1-(4-chlorophenyl)ethyl]-6-cyclobutyl-1-ethyl-4-(tetrahydro-2H-pyran- |
| | 4-ylamino)-1H-pyrazolo[3,4-b]pyridine-5-carboxamide |
| 23 | N-[1-(4-chlorophenyl)propyl]-6-cyclobutyl-1-ethyl-4-(tetrahydro-2H-pyran- |
| | 4-ylamino)-1H-pyrazolo[3,4-b]pyridine-5-carboxamide |
| 24 | 6-(cyclopropylmethyl)-1-ethyl-N-(phenylmethyl)-4-(tetrahydro-2H-pyran- |
| | 4-ylamino)-1H-pyrazolo[3,4-b]pyridine-5-carboxamide |
| 25 | 6-(cyclopropylmethyl)-N-[(3,4-dimethylphenyl)methyl]-1-ethyl-4- |
| | (tetraliydro-2H-pyran-4-ylamino)-1H-pyrazolo[3,4-b]pyridine-5- |
| | carboxamide |
| 26 | 6-(cyclopropylmethyl)-N-(2,3-dihydro-1H-inden-2-yl)-1-ethyl-4- |
| | (tetrahydro-2H-pyran-4-ylamino)-1H-pyrazolo[3,4-b]pyridine-5- |
| | carboxamide |
| 27 | N-[1-(4-chlorophenyl)ethyl]-6-(cyclopropylmethyl)-1-ethyl-4-(tetrahydro- |
| | 2H-pyran-4-ylamino)-1H-pyrazolo[3,4-b]pyridine-5-carboxamide |
| 28 | 6-cyclopentyl-1-ethyl-N-(phenylmethyl)-4-(tetrahydro-2H-pyran-4- |
| | ylamino)-1H-pyrazolo[3,4-b]pyridine-5-carboxamide |
| 29 | 6-cyclopentyl-N-(2,3-dihydro-1H-inden-2-yl)-1-ethyl-4-(tetrahydro-2H- |
| | pyran-4-ylamino)-1H-pyrazolo[3,4-b]pyridine-5-carboxamide |

### Example 1

### N-[(4-chloro-2-methylphenyl)methyl]-6-cyclopropyl-1-ethyl-4-(tetrahydro-2H-pyran-4-ylamino)-1H-pyrazolo[3,4-b]pyridine-5-carboxamide

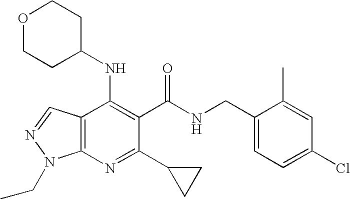

A solution of Intermediate 20 (30 mg) in DMF (2 ml) was treated with HATU (35 mg) and DIPEA (100 μL). The solution was allowed to stand at 22° C. for 10 min. then treated with 2-methyl-4-chloro benzylamine (supplier: Matrix Scientific) (14 mg) and allowed to stand at 22° C. for 16 h. The solvent was evaporated and the residue was partitioned between DCM (5 ml) and saturated aqueous NaHCO₃ (2 ml). The organic phase was collected through a hydrophobic frit and evaporated. The residue was purified by mass directed autoprep. HPLC to give Example 1 as a white solid (14.9 mg). LCMS showed MH⁺=468; T_{RET}=3.45 min.

The following Examples 2-6 were prepared from Intermediate 20 and the appropriate amine WNH₂ using a similar procedure to that used for the preparation of Example 1:

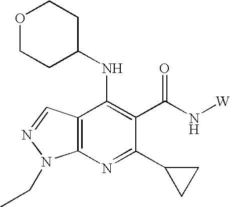

| Exam- | | Source of amine | MH⁺ | T_{RET} |
|---|---|---|---|---|
| ple No. | NHW | reagent WNH₂ | ion | (min) |
| 2 | 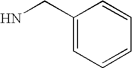 | Aldrich | 420 | 3.02 |
| 3 | 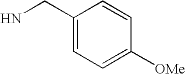 | Aldrich | 450 | 2.99 |
| 4 | 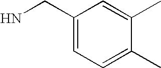 | Trans World Chemicals | 448 | 3.27 |
| 5 | 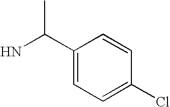 | Bionet Research | 468 | 3.39 |
| 6 | 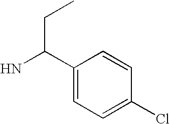 | J. Pharm. Pharmacol; 1997, 49 (1), 10- 15 | 482 | 3.51 |

The following Examples 7-11 were prepared from Intermediate 21 and the appropriate amine WNH₂ using a similar procedure to that used for the preparation of Example 1:

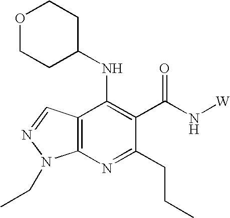

| Exam- | | Source of amine | MH⁺ | T_{RET} |
|---|---|---|---|---|
| ple No. | NHW | reagent WNH₂ | ion | (min) |
| 7 | 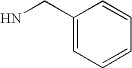 | Aldrich | 422 | 2.71 |
| 8 | 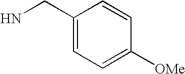 | Aldrich | 452 | 2.7 |
| 9 | 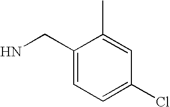 | Matrix Scientific | 470 | 3.09 |
| 10 | 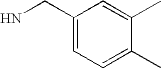 | Trans World Chemicals | 450 | 2.99 |
| 11 | 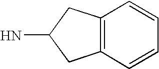 | Aldrich | 448 | 2.86 |

The following Examples 12-16 were prepared from Intermediate 22 and the appropriate amine WNH₂ using a similar procedure to that used for the preparation of Example 1:

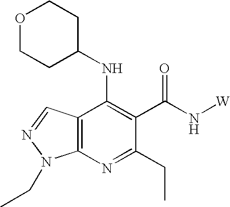

| Exam- | | Source of amine | MH⁺ | T_{RET} |
|---|---|---|---|---|
| ple No. | NHW | reagent WNH₂ | ion | (min) |
| 12 | 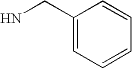 | Aldrich | 408 | 2.43 |
| 13 | 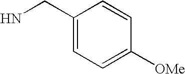 | Aldrich | 438 | 2.44 |
| 14 | 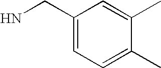 | Trans World Chemicals | 436 | 2.75 |
| 15 | 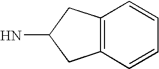 | Aldrich | 434 | 2.6 |
| 16 | 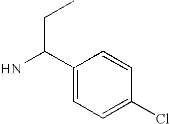 | J. Pharm. Pharmacol; 1997, 49 (1), 10- 15 | 470 | 2.94 |

The following examples 17-23 were prepared from Intermediate 23 and the appropriate amine WNH₂ using a similar procedure to that used for the preparation of Example 1:

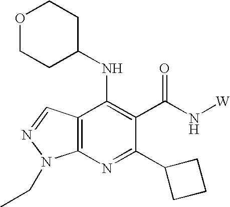

| Exam- | | Source of amine | MH⁺ | T_{RET} |
|---|---|---|---|---|
| ple No. | NHW | reagent WNH₂ | ion | (min) |
| 17 | 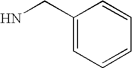 | Aldrich | 434 | 3.17 |
| 18 | 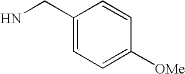 | Aldrich | 464 | 3.14 |
| 19 | 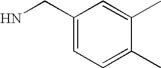 | Trans World Chemicals | 462 | 3.42 |
| 20 | 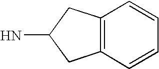 | Aldrich | 460 | 3.34 |
| 21 | 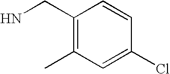 | Matrix Scientific | 482 | 3.51 |
| 22 | 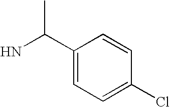 | Bionet Research | 482 | 3.45 |
| 23 | 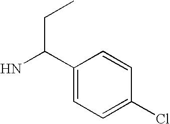 | J. Pharm. Pharmacol; 1997, 49 (1), 10- 15 | 496 | 3.57 |

The following examples 24-27 were prepared from Intermediate 24 and the appropriate amine WNH₂ using a similar procedure to that used for the preparation of Example 1:

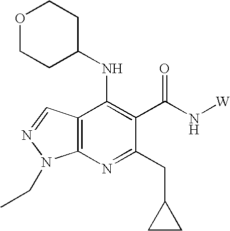

| Exam- | | Source of amine | MH⁺ | T_{RET} |
|---|---|---|---|---|
| ple No. | NHW | reagent WNH₂ | ion | (min) |
| 24 | 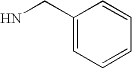 | Aldrich | 434 | 2.77 |
| 25 | 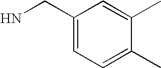 | Trans World Chemicals | 462 | 3.04 |
| 26 | 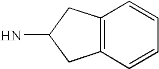 | Aldrich | 460 | 2.93 |
| 27 | 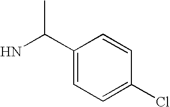 | Bionet Research | 482 | 3.09 |

The following Examples 28-29 were prepared from Intermediate 24 and the appropriate amine WNH₂ using a similar procedure to that used for the preparation of Example 1:

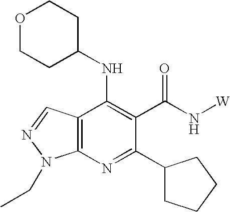

| | | Source of amine | MH⁺ | T_{RET} |
|---|---|---|---|---|
| Example No. | NHW | reagent WNH₂ | ion | (min) |
| 28 | 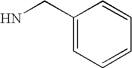 | Aldrich | 448 | 3.33 |
| 29 | 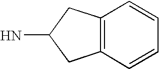 | Aldrich | 474 | 3.45 |

1. A compound of formula (I) or a salt thereof: 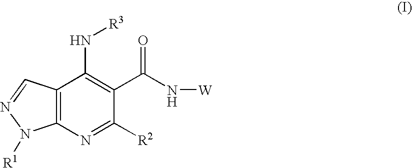
wherein:
W is Ar, —CR⁴R⁵Ar or a group (y) or (y1) wherein: 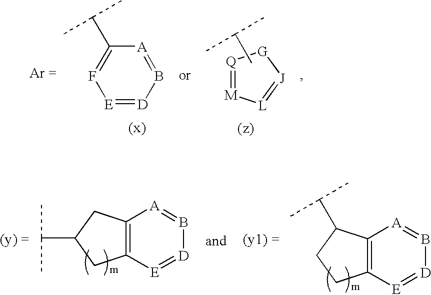
wherein m is 1 or 2;
R¹ is C₁₋₄alkyl, C₁₋₃fluoroalkyl, or —CH₂CH₂OH;
R² is C₂₋₆alkyl, C₃₋₆cycloalkyl or —(CH₂)ₙ⁴C₃₋₆cycloalkyl, wherein n⁴ is 1 or 2;
R³ is optionally substituted C₃₋₈cycloalkyl or optionally substituted mono-unsaturated-C₅₋₇cycloalkenyl or an optionally substituted heterocyclic group of sub-formula (aa), (bb) or (cc); 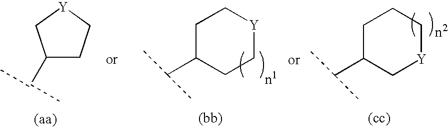
in which n¹ and n² independently are 1 or 2; and in which Y is O, S, SO₂, or NR¹⁰; where R¹⁰ is hydrogen, C₁₋₂alkyl, C₁₋₂fluoroalkyl, CH₂C(O)NH₂, C(O)NH₂, C(O)NHMe, C(O)—C₁₋₂alkyl, C(O)—C₁fluoroalkyl or —C(O)—CH₂O—C₁₋₂alkyl;
and wherein in R³ the C₃₋₈cycloalkyl or the heterocyclic group of sub-formula (aa), (bb) or (cc) is optionally substituted on a ring carbon with one or two substituents independently which are oxo (═O); OH; C₁₋₂alkoxy; C₁₋₂fluoroalkoxy; NHR²¹ wherein R²¹ is a hydrogen atom (H) hydrogen or C₁₋₄ straight-chain alkyl; C₁₋₂alkyl; C₁₋₂fluoroalkyl; —CH₂OH; —CH₂CH₂OH; —CH₂NHR²² wherein R²² is H or C₁₋₂alkyl; —C(O)OR²³ wherein R²³ is H or C₁₋₂alkyl; —C(O)NHR²⁴ wherein R²⁴ is H or C₁₋₂alkyl; —C(O)R²⁵ wherein R²⁵ is C₁₋₂alkyl; fluoro; hydroxyimino (═N—OH); or ═N—OR²⁶ where R²⁶ is C₁₋₄alkyl; and wherein any OH, alkoxy, fluoroalkoxy or NHR²¹ substituent is not substituted at the R³ ring carbon attached to the —NH— group of formula (I) and is not substituted at either R³ ring carbon bonded to the Y group of the heterocyclic group (aa), (bb) or (cc);
and wherein, when R³ is optionally substituted mono-unsaturated-C₅₋₇cycloalkenyl, then the cycloalkenyl is optionally substituted with one substituent which is fluoro or C₁₋₂alkyl or two substituents independently which are fluoro or methyl, and the R³ ring carbon bonded to the —NH— group of formula (I) does not partake in the cycloalkenyl double bond;
or R³ is a bicyclic group of sub-formula (ee): 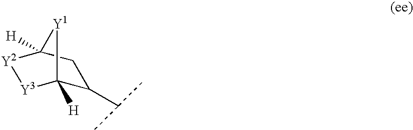
wherein Y¹, Y² and Y³ independently are CH₂ or oxygen (O) provided that no more than one of Y¹, Y² and Y³ is oxygen (O);
and wherein:
R⁴ and R⁵ are independently hydrogen, methyl, ethyl, n-propyl, isopropyl, C₁₋₂fluoroalkyl, cyclopropyl, —CH₂OR^{4a}, —CH(Me)OR^{4a}, or —CH₂CH₂OR^{4a}, wherein R^{4a} is hydrogen, methyl (Me), or C₁fluoroalkyl such as CF₃ or CHF₂.
and wherein, in sub-formula (x) (y) and (y1):
A is C—R^{6A}, nitrogen or nitrogen-oxide,
B is C—R^{6B}, nitrogen or nitrogen-oxide,
D is C—R^{6D}, nitrogen or nitrogen-oxide,
E is C—R^{6E}, nitrogen or nitrogen-oxide,
F is C—R^{6F}, nitrogen or nitrogen-oxide,
wherein, R^{6A}, R^{6B}, R^{6D}, R^{6E} and R^{6F} independently are: hydrogen, a halogen atom; C₁₋₆alkyl; C₁₋₄fluoroalkyl; C₃₋₆cycloalkyl; C₁₋₄alkoxy; C₁₋₂fluoroalkoxy; C₃₋₆cycloalkyloxy; —C(O)R^{16a}; —C(O)OR³⁰; —S(O)₂—R^{16a}; R^{16a}—S(O)₂—NR^{15a}—; R⁷R⁸N—S(O)₂—; C₁₋₂alkyl-C(O)—R^{15a}N—S(O)₂—; C₁₋₄alkyl-S(O)—, Ph-S(O)—, R⁷R⁸N—CO—; —NR¹⁵—C(O)R^{16a}; R⁷R⁸N; nitro (—NO₂); OH; C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂—OH; —CH₂—NR⁷R⁸; —CH₂—CH₂—NR⁷R⁸; —CH₂—C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano (—CN); Ar^{5b}; or phenyl, pyridinyl or pyrimidinyl wherein the phenyl, pyridinyl or pyrimidinyl independently are optionally substituted by one or two groups which are fluoro, chloro, C₁₋₂alkyl, C₁fluoroalkyl, C₁₋₂alkoxy or C₁fluoroalkoxy;
and/or two adjacent groups selected from the group consisting of R^{6A}, R^{6B}, R^{6D}, R^{6E} and R^{6F} are taken together and are: —CH═CH—CH═CH₂—, —(CH₂)ₙ^{14a}— where n^{14a} is 3, 4 or 5, —O—(CMe₂)-O—, —O—(CH₂)ₙ^{14b}—O— where n¹⁴is 1 or 2; —CH═CH—NR^{15b}—; —N═CH—NR^{15b}—; —CH═N—NR^{15b}—; —N═N—NR^{15b}—; —CH═CH—O—; —N═CH—O—; —CH═CH—S—; or —N═CH—S—; wherein R^{15b} is H or C₁₋₂alkyl;
provided that:
two or more of A, B, D, E and F are independently C—H, C—F, nitrogen , or nitrogen-oxide;
and no more than two of A, B, D, E and F are independently nitrogen or nitrogen-oxide, and no more than one of A, B, D, E and F is nitrogen-oxide;
and wherein, in sub-formula (z):
G is O or S or NR⁹ wherein R⁹ is hydrogen, C₁₋₄alkyl, or
C₁₋₂fluoroalkyl;
J is C—R^{6J}, C-[connection point to formula (I)], or nitrogen,
L is C—R^{6L}, C-[connection point to formula (I)], or nitrogen,
M is C—R^{6M}, C-[connection point to formula (I)], or nitrogen),
Q is C—R^{6Q}, C-[connection point to formula (I)], or nitrogen),
wherein, R^{6J}, R⁶L, R⁶M and R^{6Q} independently are: hydrogen, a halogen atom; C₁₋₄alkyl; C₁₋₃fluoroalkyl; C₃₋₆cycloalkyl; C₁₋₄alkoxy; C₁₋₂fluoroalkoxy; C₃₋₆cycloalkyloxy; OH (including any tautomer thereof); or phenyl optionally substituted by one or two substituents independently being fluoro, chloro, C₁₋₂alkyl, C₁fluoroalkyl, C₁₋₂alkoxy or C₁fluoroalkoxy;
provided that:
two or more of J, L, M and Q are independently C—H, C—F, C—C₁₋₂alkyl, C-[connection point to formula (I)], or nitrogen;
and no more than three of J, L, M and Q are nitrogen;
and wherein:
R⁷ and R⁸ are independently hydrogen; C₁₋₄alkyl; C₃₋₆cycloalkyl; or phenyl optionally substituted by one or two substituents independently being: fluoro, chloro, C₁₋₂alkyl, C₁fluoroalkyl, C₁₋₂alkoxy or C₁fluoroalkoxy;
or R⁷ and R⁸ together are —(CH₂)ₙ⁶— or —C(O)—(CH₂)ₙ⁷— or —C(O)—(CH₂)ₙ¹⁰—C(O)—or —(CH₂)ₙ⁸—X⁷—(CH₂)ₙ⁹— or —C(O)—X⁷—(CH₂)ₙ¹⁰— in which: n⁶ is 3, 4, 5 or 6, n⁷ is 2, 3, 4, or 5, n⁸ and n⁹ and n¹⁰ independently are 2 or 3, and X⁷ is O or NR¹⁴;
R^{7a} is hydrogen or C₁₋₄alkyl;
R^{8a} is (H) hydrogen or methyl;
R¹⁴, R¹⁷ and R^{17a} independently are: hydrogen; C₁₋₄alkyl; C₁₋₂fluoroalkyl (e.g. CF₃); cyclopropyl; —C(O)—C₁₋₄alkyl; —C(O)NR^{7a}R^{8a}; or —S(O)₂—C₁₋₄alkyl;
R^{15a}, independent of other R^{15a}, is hydrogen or C₁₋₄alkyl;
R^{16a} is:
C₁₋₆alkyl;
C₃₋₆cycloalkyl optionally substituted by one oxo (═O), OH or C₁₋₂alkyl substituent;
C₃₋₆cycloalkyl-CH₂—;
pyridinyl optionally substituted on a ring carbon atom by one of: a halogen atom, C₁₋₂alkyl, C₁fluoroalkyl, C₁₋₂alkoxy or C₁fluoroalkoxy;
Ar^{5c};
phenyl optionally substituted by one or two substituents independently being: a halogen atom, C₁₋₂alkyl, C₁fluoroalkyl, C₁₋₂alkoxy or C₁fluoroalkoxy; benzyl optionally substituted on its ring by one or two substituents independently being: a halogen atom, C₁₋₂alkyl, C₁fluoroalkyl, C₁₋₂alkoxy or C₁fluoroalkoxy; or a 4-, 5-, 6- or 7-membered saturated heterocyclic ring connected at a ring-carbon and containing one or two ring-hetero-atoms independently selected from O, S, and N; wherein any ring-nitrogens which are present are present as NR²⁷ where R²⁷ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen;
R³⁰, independent of other R³⁰, hydrogen, C₁₋₄alkyl or C₃₋₆cycloalkyl;
Ar^{5b} and Ar^{5c} independently are a 5-membered aromatic heterocyclic ring containing one O, S or NR^{15a} in the 5-membered ring, wherein the 5-membered ring can optionally additionally contain one or two N atoms, and wherein the heterocyclic ring is optionally substituted on a ring carbon atom by one of: halo, C₁₋₂alkyl, C₁fluoroalkyl, —CH₂OH, —CH₂—OC₁₋₂alkyl, OH or —CH₂—NR²⁸R²⁹ wherein R²⁸ and R²⁹ independently are H or methyl; and
Het¹ is a 4-, 5-, 6- or 7-membered saturated heterocyclic ring connected at a ring-carbon and containing one or two ring-hetero-atoms independently selected from the group consisting of O, S, and N; wherein any ring-nitrogens which are present are present as NR³¹ where R³¹ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen.

2. A compound or salt as claimed in claim 1, wherein R¹ is C₂₋₃alkyl, C₂fluoroalkyl or —CH₂CH₂OH.

3. A compound or salt as claimed in claim 2, wherein R¹ is ethyl, n-propyl or —CH₂CH₂OH.

4. A compound or salt as claimed in claim 3, wherein R¹ is ethyl.

5. A compound or salt as claimed in claim 1, wherein R² is C₂₋₄alkyl, C₃₋₅cycloalkyl or —CH₂cyclopropyl.

6. A compound or salt as claimed in claim 5, wherein R² is ethyl, propyl, cyclopropyl, cyclobutyl, cyclopentyl or cyclopropylmethyl.

7. A compound or salt as claimed in claim 1 wherein in R³ there is one substituent or no substituent.

8. A compound or salt as claimed in claim 1, wherein R³ is the optionally substituted C₃₋₈cycloalkyl or the optionally substituted heterocyclic group of sub-formula (aa), (bb) or (cc).

9. A compound or salt as claimed in claim 1, wherein, when R³ is optionally substituted C₃₋₈cycloalkyl, it is optionally substituted cyclohexyl.

10. A compound or salt as claimed in claim 1, wherein, when R³ is optionally substituted C₃₋₈cycloalkyl, then R³ is C₆₋₇cycloalkyl optionally substituted with one or two substituents independently selected from the group consisting of oxo (═O); OH; NHR²¹ wherein R²¹ is hydrogen; methyl; —CH₂F; —CHF₂; —C(O)OR²³ wherein R²³ is H; —C(O)NHR²⁴ wherein R²⁴ is H; fluoro; hydroxyimino (═N—OH); and methoxyimino (═N—OR²⁶ where R²⁶ is methyl).

11. A compound or salt as claimed in claim 10, wherein, when R³ is optionally substituted C₃₋₈cycloalkyl, then R³ is C₆₋₇cycloalkyl optionally substituted with one or two substituents independently selected from the group consisting of OH; —C(O)NHR²⁴ wherein R²⁴ is H; oxo and hydroxyimino.

12. A compound or salt as claimed in claim 1 wherein, for R³, the one or two optional R³ substituents if present are:
(a) at the 3-position of a R³ cyclobutyl ring, or
(b) at the 3- and/or 4-position(s) of a R³ cyclopentyl or cyclopentenyl ring, or
(c) at the 3-, 4- and/or 5-position(s) of a R³ cyclohexyl or cyclohexenyl ring, or
(d) at the 3-, 4-, 5- and/or 6-position(s) of a R³ cycloheptyl or cycloheptenyl ring, or
(e) at the 3-, 4-, 5-, 6- and/or 7-position(s) of a R³ cyclooctyl ring, or
(f) at the 1-, 2- and/or highest-numbered-position(s) of a R³ cycloalkyl or cycloalkenyl ring, for alkyl or fluoroalkyl substituent(s), or
(g) at the 2- or highest-numbered-position(s) of a R³ cycloalkyl or cycloalkenyl ring, for NHR²¹ substituent(s).

13. A compound or salt as claimed in claim 1 wherein, when R³ is the heterocyclic group of sub-formula (aa), (bb) or (cc), then Y is O or NR¹⁰.

14. A compound or salt as claimed in claim 1 wherein R¹⁰ is H, C(O)NH₂ or C(O)methyl.

15. A compound or salt as claimed in claim 14, wherein R¹⁰ is C(O)NH₂.

16. A compound or salt as claimed in claim 1 wherein, when R³ is the heterocyclic group of sub-formula (aa), (bb) or (cc), then R³ is the heterocyclic group of sub-formula (bb) and n¹ is 1.

17. (canceled)

18. A compound or salt as claimed in claim 1 wherein:
when R³ is optionally substituted mono-unsaturated-C₅₋₇cycloalkenyl, it is mono-unsaturated-cyclohexenyl optionally substituted with one or two substituents which are fluoro or methyl
and when R³ is a bicyclic group of sub-formula (ee), then Y¹, Y² and Y³ are all CH₂.

19. A compound or salt as claimed in claim 1 wherein NHR³ is of sub-formula (a), (a1), (b), (c), (c 1), (c 2), (c 3), (c 4), (c 5), (c 6), (c 7), (d), (e), (f), (g), (g1), (g2), (g3), (g4), (h), (i), (j), (k), (k1), (k2), (L), (m), (m1), (m2), (m3), (m5), (n), (o), (o1), (o2), (o3), (p), (p1), (p2), (p3), (p4), (p5), (p6), (p7), (p8), (p9), (p10), (p11) or (q):

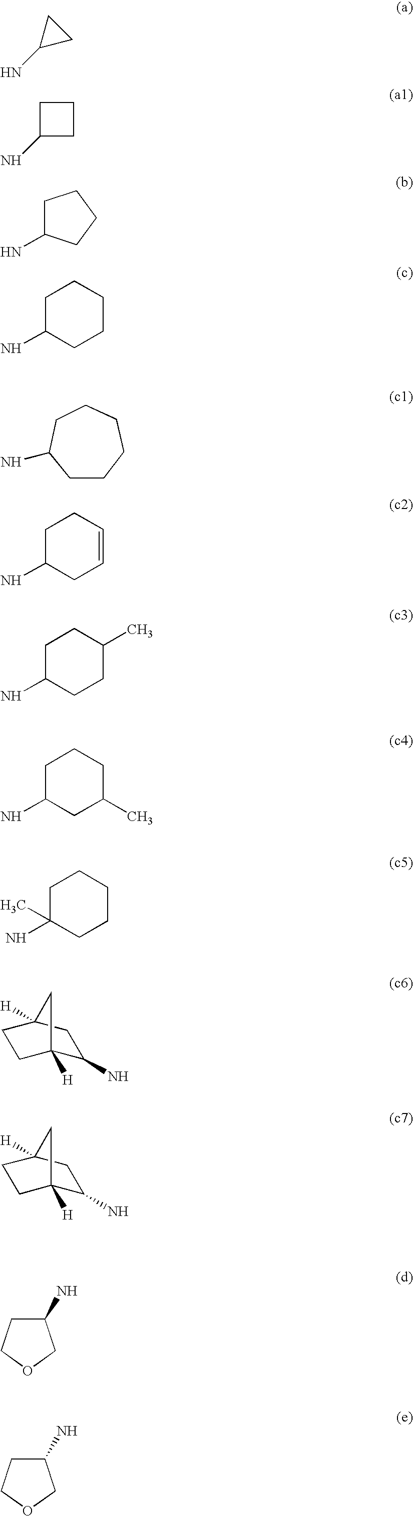

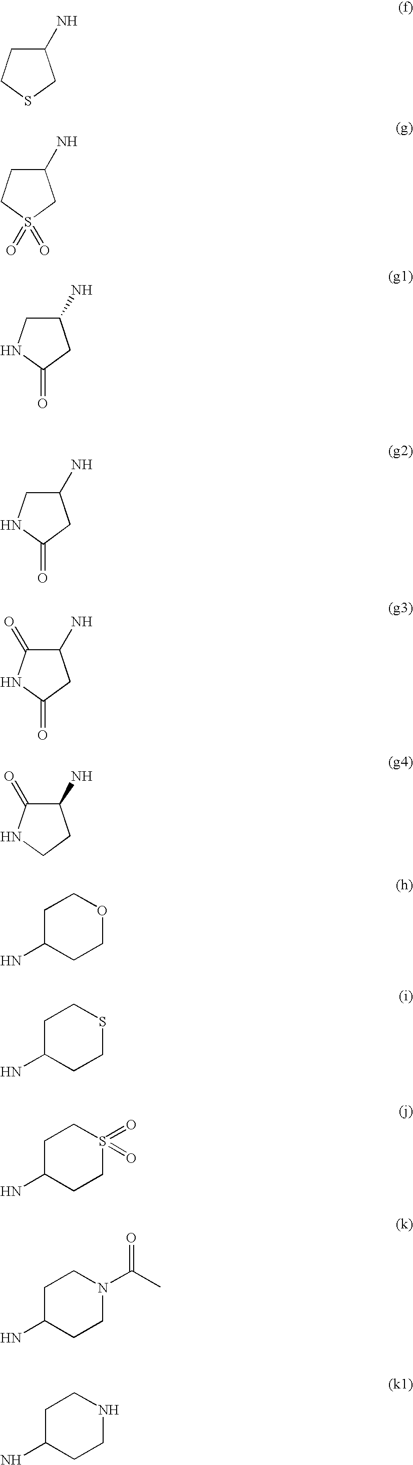

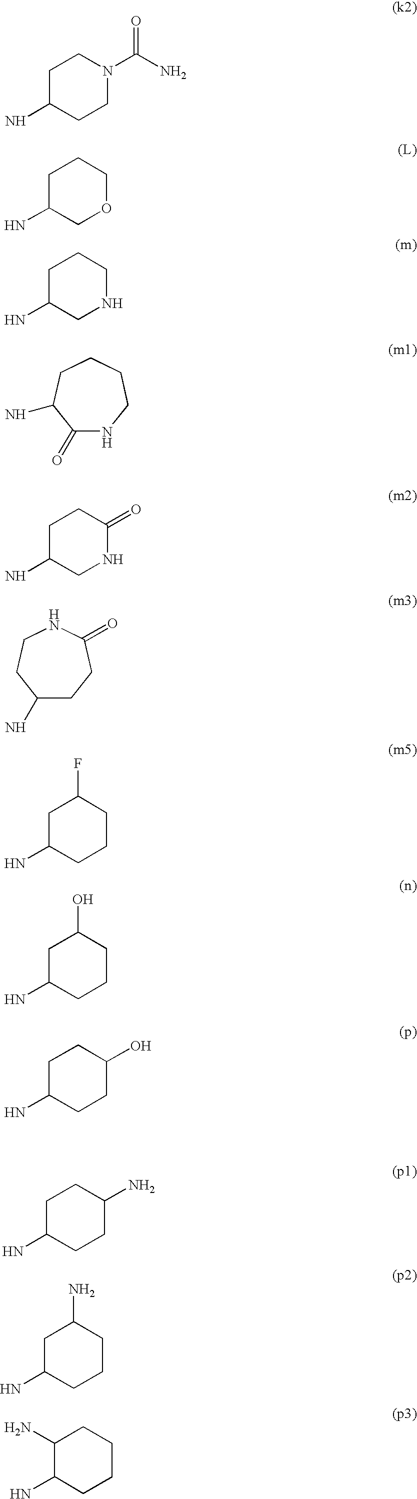

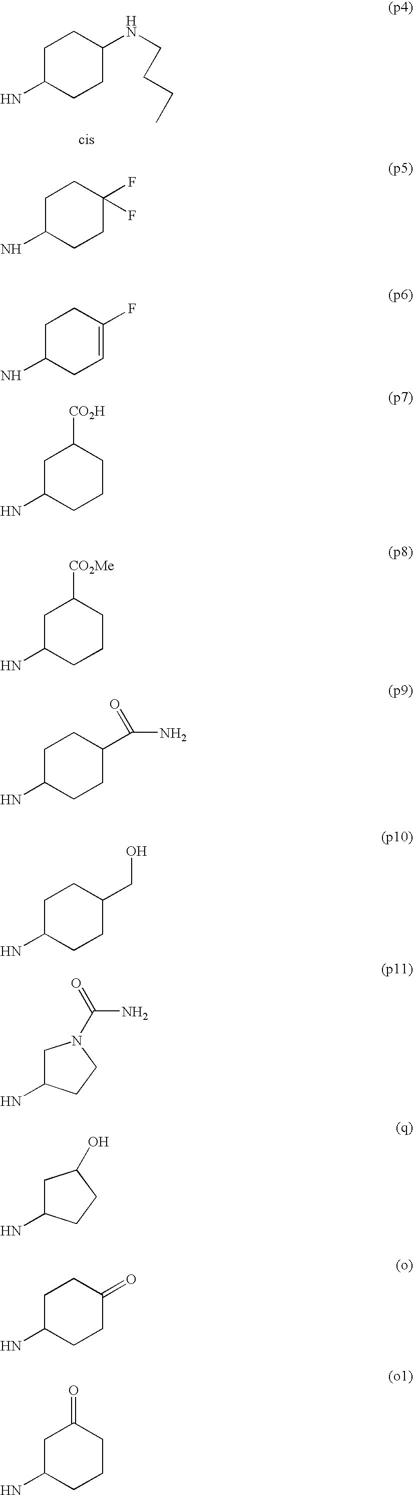

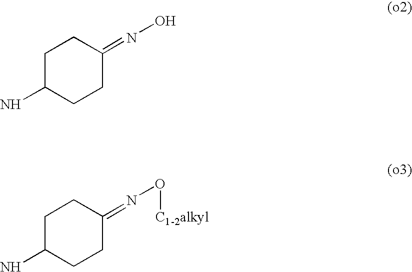

20. A compound or salt as claimed in claim 19, wherein NHR³ is sub-formula (c), (c1), (c 4), (c 5), (h), (i), (j), (k), (k2), (m1), (m2), (n), (o), (o2), (o3), (p2) (p5), (p6), (p9), (p11) or (q).

21. A compound or salt as claimed in claim 19, wherein NHR³ is sub-formula (c), (p11), (h), (k2), (n), (o), (o2) or (p9).

22. A compound or salt as claimed in claim 19, wherein:
when NHR³ is sub-formula (n), then it is in the cis configuration; and
when NHR³ is sub-formula (p9), then it is in the cis configuration.

23. A compound or salt as claimed in claim 19, wherein NHR³ is sub-formula (h) or (k2), that is R³ is tetrahydro-2H-pyran-4-yl or 1-(aminocarbonyl)-4-piperidinyl.

24. A compound or salt as claimed in claim 1 wherein R⁴ is hydrogen, methyl, ethyl, C₁fluoroalkyl, —CH₂OH, —CH(Me)OH, —CH₂CH₂OH, or —CH₂OMe.

25. A compound or salt as claimed in claim 24, wherein R⁴ is hydrogen, methyl, ethyl, —CH₂OH, or —CH₂OMe.

26. A compound or salt as claimed in claim 1 wherein R⁵ is hydrogen, methyl, ethyl, n-propyl, or iso-propyl.

27. A compound or salt as claimed in claim 1 wherein, in sub-formula (x):
two or more of A, B, D, E and F are C—H; and one or more others of A, B, D, E and F are independently C—H, C—F, C—Cl, C-Me, C—OMe, or nitrogen;
no more than one of A, B, D, E and F is nitrogen; and
excluding compounds where A, B, D, E and F are nitrogen-oxide (N⁺—O⁻).

28. A compound or salt as claimed in claim 1 wherein Ar is the sub-formula (x).

29. A compound or salt as claimed in claim 28, wherein is sub-formula (x1), (x2), (x3), (x4), (x5), (x6), (x7), (x8), (x9), (x10), (x11), (x12), (x13), (x14), (x15) or (x16):

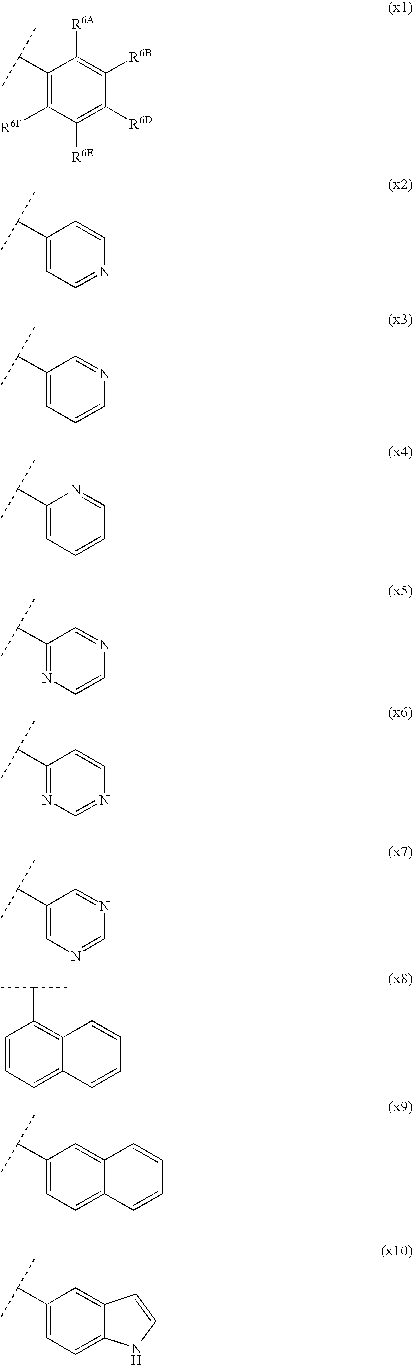

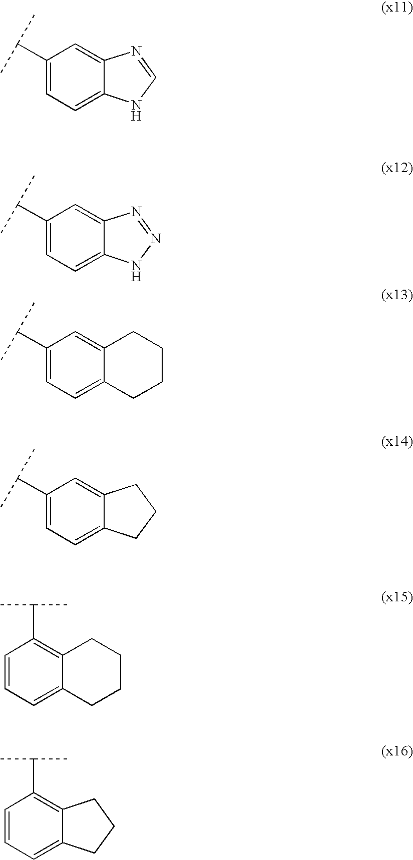

30. A compound or salt as claimed in claim 29, wherein Ar is sub-formula (x1).

31. A compound or salt as claimed in claim 30, wherein Ar is: monoalkyl-phenyl-, mono(fluoroalkyl)-phenyl-, monohalo-phenyl-, monoalkoxy-phenyl-, mono(fluoroalkoxy)-phenyl-, dialkyl-phenyl-, monoalkyl-monohalo-phenyl-, dihalo-phenyl- or dihalo-monoalkyl-phenyl-.

32. A compound or salt as claimed in claim 31, wherein Ar is: monoC₁₋₄alkyl-phenyl-; monoC₁fluoroalkyl-phenyl-; monoC₁₋₃alkoxy-phenyl-; mono(C₁fluoroalkoxy)-phenyl-; diC₁₋₃alkyl-phenyl-; monoC₁₋₃alkyl-monohalo-phenyl-; dihalo-phenyl-; or dihalo-monoC₁₋₂alkyl-phenyl-.

33. A compound or salt as claimed in claim 1 wherein, in sub-formula (x), R^{6A}, R^{6B}, R^{6D}, R^{6E} and R^{6F}, independently of each other, are: hydrogen, a fluorine, chlorine or bromine atom, methyl, ethyl, n-propyl, isopropyl, trifluoromethyl, —CH₂OH, methoxy, ethoxy, n-propoxy, difluoromethoxy, OH or MeS(O)₂—.

34. A compound or salt as claimed in claim 1 wherein
R⁹ is hydrogen or methyl;
R^{6J}, R^{6L}, R^{6M} and R^{6Q} independently are H, OH C₁₋₂alkyl or C₁fluoroalkyl; and
when Ar has the sub-formula (z), then sub-formula (z) is:

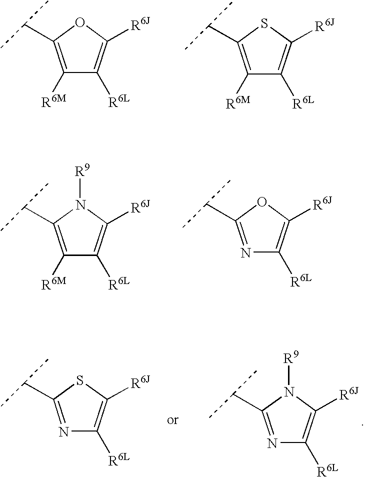

35. A compound or salt as claimed in claim 1, which is
N-[(4-chloro-2-methylphenyl)methyl]-6-cyclopropyl-1-ethyl-4-(tetrahydro-2H-pyran-4-ylamino)-1H-pyrazolo[3,4-b]pyridine-5-carboxamide:
N-[(4-chloro-2-methylphenyl)methyl]-6-cyclopropyl-1-ethyl-4-(tetrahydro-2H-pyran-4-ylamino)-1H-pyrazolo[3,4-b]pyridine-5-carboxamide:
6-cyclopropyl-1-ethyl-N-(phenylmethyl)-4-(tetrahydro-2H-pyran-4-ylamino)-1H-pyrazolo[3,4-b]pyridine-5-carboxamide:
6-cyclopropyl-1-ethyl-N-{[4-(methyloxy)phenyl]methyl}-4-(tetrahydro-2H-pyran-4-ylamino)-1H-pyrazolo[3,4-b]pyridine-5-carboxamide:
6-cyclopropyl-N-[(3,4-dimethylphenyl)methyl]-1-ethyl-4-(tetrahydro-2H-pyran-4-ylamino)-1H-pyrazolo[3,4-b]pyridine-5-carboxamide:
N-[1-(4-chlorophenyl)ethyl]-6-cyclopropyl-1-ethyl-4-(tetrahydro-2H-pyran-4-ylamino)-1H-pyrazolo[3,4-b]pyridine-5-carboxamide:
N-[1-(4-chlorophenyl)propyl]-6-cyclopropyl-1-ethyl-4-(tetrahydro-2H-pyran-4-ylamino)-1H-pyrazolo[3,4-b]pyridine-5-carboxamide;
1-ethyl-N-(phenylmethyl)-6-propyl-4-(tetrahydro-2H-pyran-4-ylamino)-1H-pyrazolo[3,4-b]pyridine-5-carboxamide:
1-ethyl-N-{[4-(methyloxy)phenyl]methyl}-6-propyl-4-(tetrahydro-2H-pyran-4-ylamino)-1H-pyrazolo[3,4-b]pyridine-5-carboxamide:
N-[(4-chloro-2-methylphenyl)methyl]-1-ethyl-6-propyl-4-(tetrahydro-2H-pyran-4-ylamino)-1H-pyrazolo[3,4-b]pyridine-5-carboxamide;
N-[(3,4-dimethylphenyl)methyl]-1-ethyl-6-propyl-4-(tetrahydro-2H-pyran-4-ylamino)-1H-pyrazolo[3,4-b]pyridine-5-carboxamide:
N-(2,3-dihydro-1H-inden-2-yl)-1-ethyl-6-propyl-4-(tetrahydro-2H-pyran-4-ylamino)-1H-pyrazolo[3,4-b]pyridine-5-carboxamide:
1,6-diethyl-N-(phenylmethyl)-4-(tetrahydro-2H-pyran-4-ylamino)-1H-pyrazolo[3,4-b]pyridine-5-carboxamide:
1,6-diethyl-N-{[4-(methyloxy)phenyl]methyl}-4-(tetrahydro-2H-pyran-4-ylamino)-1H-pyrazolo[3,4-b]pyridine-5-carboxamide:
N-[(3,4-dimethylphenyl)methyl]-1,6-diethyl-4-(tetrahydro-2H-pyran-4-ylamino)-1H-pyrazolo[3,4-b]pyridine-5-carboxamide;
N-(2,3-dihydro-1H-inden-2-yl)-1,6-diethyl-4-(tetrahydro-2H-pyran-4-ylamino)-1H-pyrazolo[3,4-b]pyridine-5-carboxamide:
N-[1-(4-chlorophenyl)propyl]-1,6-diethyl-4-(tetrahydro-2H-pyran-4-ylamino)-1H-pyrazolo[3,4-b]pyridine-5-carboxamide:
6-cyclobutyl-1-ethyl-N-(phenylmethyl)-4-(tetrahydro-2H-pyran-4-ylamino)-1H-pyrazolo[3,4-b]pyridine-5-carboxamide:
6-cyclobutyl-1-ethyl-N-{[4-(methyloxy)phenyl]methyl}-4-(tetrahydro-2H-pyran-4-ylamino)-1H-pyrazolo[3,4-b]pyridine-5-carboxamide:
6-(cyclopropylmethyl)-N-[(3,4-dimethylphenyl)methyl]-1-ethyl-4-(tetrahydro-2H-pyran-4-ylamino)-1H-pyrazolo[3,4-b]pyridine-5-carboxamide;
6-cyclobutyl-N-(2,3-dihydro-1H-inden-2-yl)-1-ethyl-4-(tetrahydro-2H-pyran-4-ylamino)-1H-pyrazolo[3,4-b]pyridine-5-carboxamide:
N-[(4-chloro-2-methylphenyl)methyl]-6-cyclobutyl-1-ethyl-4-(tetrahydro-2H-pyran-4-ylamino)-1H-pyrazolo[3,4-b]pyridine-5-carboxamide:
N-[1-(4-chlorophenyl)ethyl]-6-cyclobutyl-1-ethyl-4-(tetrahydro-2H-pyran-4-ylamino)-1H-pyrazolo[3,4-b]pyridine-5-carboxamide:
N-[1-(4-chlorophenyl)propyl]-6-cyclobutyl-1-ethyl-4-(tetrahydro-2H-pyran-4-ylamino)-1H-pyrazolo[3,4-b]pyridine-5-carboxamide:
6-(cyclopropylmethyl)-1-ethyl-N-(phenylmethyl)-4-(tetrahydro-2H-pyran-4-ylamino)-1H-pyrazolo[3,4-b]pyridine-5-carboxamide;
6-(cyclopropylmethyl)-N-[(3,4-dimethylphenyl)methyl]-1-ethyl-4-(tetrahydro-2H-pyran-4-ylamino)-1H-pyrazolo[3,4-b]pyridine-5-carboxamide:
6-(cyclopropylmethyl)-N-(2,3-dihydro-1H-inden-2-yl)-1-ethyl-4-(tetrahydro-2H-pyran-4-ylamino)-1H-pyrazolo[3,4-b]pyridine-5-carboxamide:
N-[1-(4-chlorophenyl)ethyl]-6-(cyclopropylmethyl)-1-ethyl-4-(tetrahydro-2H-pyran-4-ylamino)-1H-pyrazolo[3,4-b]pyridine-5-carboxamide;
6-cyclopentyl-1-ethyl-N-(phenylmethyl)-4-(tetrahydro-2H-pyran-4-ylamino)-1H-pyrazolo[3,4-b]pyridine-5-carboxamide: and
6-cyclopentyl-N-(2,3-dihydro-1H-inden-2-yl)-1-ethyl-4-(tetrahydro-2H-pyran-4-ylamino)-1H-pyrazolo[3,4-b]pyridine-5-carboxamide.

36. (canceled)

37. A pharmaceutical composition comprising a compound of formula (I) or a pharmaceutically acceptable salt thereof, as defined in claim 1 and one or more pharmaceutically acceptable carriers and/or excipients.

38-39. (canceled)

40. A method of treatment and/or prophylaxis of an inflammatory and/or allergic disease in a human in need thereof, which method comprises administering to the human a therapeutically effective amount of a compound of formula (I) or a pharmaceutically acceptable salt thereof as defined in claim 1.
